(19) United States
(12) Patent Application Publication
(10) Pub. No.: US 2023/0348593 A1
(43) Pub. Date: Nov. 2, 2023

(54) ANTIBODY MOLECULES THAT BIND TO NKP30 AND USES THEREOF

(71) Applicant: Marengo Therapeutics, Inc., Cambridge, MA (US)

(72) Inventors: Andreas Loew, Boston, MA (US); Madan Katragadda, Acton, MA (US); Andrea Tiffany, Cambridge, MA (US)

(21) Appl. No.: 18/172,928

(22) Filed: Feb. 22, 2023

Related U.S. Application Data
(63) Continuation of application No. PCT/US2021/047574, filed on Aug. 25, 2021.
(60) Provisional application No. 63/070,782, filed on Aug. 26, 2020.

(51) Int. Cl. C07K 16/28; C07K 14/54

(52) U.S. Cl. CPC C07K 16/2803; C07K 14/54; C07K 14/5418; C07K 14/5434; C07K 14/5443; C07K 2317/31; C07K 2319/30

(57) ABSTRACT

Antibody molecules that specifically bind to NKp30 are disclosed. The anti-NKp30 antibody molecules can be used to treat, prevent and/or diagnose cancerous, autoimmune or infectious conditions and disorders.

### RELATED APPLICATIONS

This application is a continuation of International Application No. PCT/US2021/047574, filed Aug. 25, 2021, which claims the benefit of U.S. Provisional Application No. 63/070,782, filed on Aug. 26, 2020, the entire contents of which are hereby incorporated by reference.

### REFERENCE TO A SEQUENCE LISTING XML

This application contains a Sequence Listing which has been submitted electronically in XML format. The Sequence Listing XML is incorporated herein by reference. Said XML file, created on Feb. 15, 2023, is named 53676-741_301_SL.xml and is 1,794,186 bytes in size.

### BACKGROUND

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46.

Given the importance of immune checkpoint pathways in regulating an immune response, the need exists for developing novel agents that modulate the activity of immunoinhibitory proteins, such as PD-1, thus leading to activation of immune system. Such agents can be used, e.g., for cancer immunotherapy and treatment of other conditions, such as chronic infection.

### SUMMARY OF THE INVENTION

Provided herein, inter alia, in an aspect, is an isolated antibody molecule that binds to NKp30, comprising:
(a) a heavy chain complementarity determining region 1 (VHCDR1), a heavy chain complementarity determining region 2 (VHCDR2), and/or a heavy chain complementarity determining region 3 (VHCDR3) described in Table 8A, or a sequence with no more than 1, 2, 3, or 4 mutations, e.g., substitutions, additions, or deletions; and/or
(b) a light chain complementarity determining region 1 (VLCDR1), a light chain complementarity determining region 2 (VLCDR2), and/or a light chain complementarity determining region 3 (VLCDR3) described in Table 8B, or a sequence with no more than 1, 2, 3, or 4 mutations, e.g., substitutions, additions, or deletions.

In some embodiments, the VHCDR1 comprises the amino acid sequence of any one of SEQ ID NOs: 19, 33, 47, 61, 7397, 89, 103, and 116, or an amino acid sequence with no more than 1, 2, 3, or 4 mutations, e.g., substitutions, additions, or deletions

In some embodiments, the VHCDR2 comprises the amino acid sequence of any one of SEQ ID NOs: 21, 35, 49, 63, 7399, 91, 105, or 118, or an amino acid sequence with no more than 1, 2, 3, or 4 mutations, e.g., substitutions, additions, or deletions.

In some embodiments, the VHCDR3 comprises the amino acid sequence of any one of SEQ ID NOs: 23, 37, 51, 65, 79, 93, 107, and 120, or an amino acid sequence with no more than 1, 2, 3, or 4 mutations, e.g., substitutions, additions, or deletions.

In some embodiments, the VLCDR1 comprises the amino acid sequence of any one of SEQ ID NOs: 26, 40, 54, 68, 82, 96, 110, and 123, or an amino acid sequence with no more than 1, 2, 3, or 4 mutations, e.g., substitutions, additions, or deletions.

In some embodiments, the VLCDR2 comprises the amino acid sequence of any one of SEQ ID NOs: 28, 7393, 56, 70, 84, 98, 112, and 125, or an amino acid sequence with no more than 1, 2, 3, or 4 mutations, e.g., substitutions, additions, or deletions.

In some embodiments, the VLCDR3 comprises the amino acid sequence of any one of SEQ ID NOs: 30, 7395, 58, 72, 86, 100, 113, and 127, or an amino acid sequence with no more than 1, 2, 3, or 4 mutations, e.g., substitutions, additions, or deletions.

In some embodiments, the antibody molecule as described herein comprises:
(a) a heavy chain framework region 1 (VHFWR1), a heavy chain framework region 2 (VHFWR1), a heavy chain framework region 3 (VHFWR3), and/or a heavy chain framework region 4 (VHFWR4) described in Table 8A, or an amino acid sequence with no more than 1, 2, 3, or 4 mutations, e.g., substitutions, additions, or deletions; and/or
(b) a light chain framework region 1 (VLFWR1), a light chain framework region 2 (VLFWR1), a light chain framework region 3 (VLFWR3), and/or a light chain framework region 4 (VLFWR4) described in Table 8B, or an amino acid sequence with no more than 1, 2, 3, or 4 mutations, e.g., substitutions, additions, or deletions.

In some embodiments, the antibody molecule as described herein comprises:
(a) a heavy chain variable region (VH) described in Table 9, e.g., a VH comprising the amino acid sequence of any one of SEQ ID NOs: 1-8, or an amino acid sequence having at least about 75%, 80%, 85%, 90%, 95%, or 99% sequence identity thereto; and/or
(b) a light chain variable region (VL) described in Table 9, e.g., a VL comprising the amino acid sequence of any one of SEQ ID NOs: 9-16, or an amino acid sequence having at least about 75%, 80%, 85%, 90%, 95%, or 99% sequence identity thereto.

In some embodiments, the antibody molecule as described herein comprises an amino acid sequence described in Table 10, e.g., the amino acid sequence of any one of SEQ ID NOs: 17, and 7386-7392, or an amino acid sequence having at least about 75%, 80%, 85%, 90%, 95%, or 99% sequence identity thereto.

In another aspect, provides herein is a composition comprising a polypeptide molecule comprising the antibody molecule as described herein.

In some embodiments, the polypeptide molecule is a multifunctional polypeptide molecule.

In some embodiments, the polypeptide molecule is a multispecific polypeptide molecule.

In some embodiments, the polypeptide molecule further comprises a targeting moiety.

In some embodiments, the targeting moiety is selected from a tumor targeting moiety, a targeting moiety that that targets an autoreactive T cell, and a targeting moiety that targets an infected cell.

In some embodiments, the targeting moiety that targets the autoreactive T cell binds to an antigen present on the surface of the autoreactive T cell.

In some embodiments, the antigen present on the surface of the autoreactive T cell is associated with an inflammatory or autoimmune disorder.

In some embodiments, the antigen present on the surface of the autoreactive T cell is selected from the group consisting of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, and CD226.

In some embodiments, the targeting moiety that targets the infected cell binds to an antigen associated with a viral infection or a bacterial infection.

In some embodiments, the polypeptide molecule further comprises a cytokine molecule.

In some embodiments, the polypeptide molecule further comprises one, two, three, four or more of:
(a) a tumor targeting moiety, e.g., as described herein;
(b) a cytokine molecule, e.g., as described herein;
(c) a T cell engager, e.g., as described herein; or
(d) a stromal modifying moiety, e.g., as described herein.

In some embodiments, the cytokine molecule is a cytokine molecule selected from the group consisting of GM-CSF, IL-1α, IL-1β, IL-2, IL-3, IL-4, IL-5, IL-6, IL-7, IL-8, IL-10, IL-12, IL-15, IL-18, IL-21, IFN-α, IFN-β, IFN-γ, MIP-1α, MIP-1β, TGF-β, TNF-α, and TNFβ.

In some embodiments, the antibody molecule is a monospecific antibody molecule, a bispecific antibody molecule, or a trispecific antibody molecule.

In some embodiments, the antibody molecule is a monovalent antibody molecule, a bivalent antibody molecule, or a trivalent antibody molecule.

In some embodiments, the antibody molecule is a full antibody (e.g., an antibody that includes at least one, and preferably two, complete heavy chains, and at least one, and preferably two, complete light chains), or an antigen-binding fragment (e.g., a Fab, F(ab′)2, Fv, a single chain Fv, a single domain antibody, a diabody (dAb), a bivalent antibody, or bispecific antibody or fragment thereof, a single domain variant thereof, or a camelid antibody).

In some embodiments, the antibody comprises a heavy chain constant region chosen from IgG1, IgG2, IgG3, or IgG4, or a fragment thereof.

In some embodiments, the antibody comprises a light chain constant region chosen from the light chain constant regions of kappa or lambda, or a fragment thereof.

In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function.

In some embodiments, an interface of a first and second immunoglobulin chain constant regions (e.g., Fc region) is altered, e.g., mutated, to increase or decrease dimerization, e.g., relative to a non-engineered interface.

In some embodiments, the dimerization of the immunoglobulin chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface.

In some embodiments, the immunoglobulin chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1.

In some embodiments, the immunoglobulin chain constant region (e.g., Fc region) comprises an amino acid substitution chosen from: T366S, L368A, or Y407V (e.g., corresponding to a cavity or hole), or T366W (e.g., corresponding to a protuberance or knob), or a combination thereof.

In some embodiments, the antibody molecule or the polypeptide molecule further comprises a linker, e.g., a linker between one or more of: the targeting moiety and the cytokine molecule or the stromal modifying moiety, the targeting moiety and the immune cell engager, the cytokine molecule or the stromal modifying moiety, and the immune cell engager, the cytokine molecule or the stromal modifying moiety and the immunoglobulin chain constant region (e.g., the Fc region), the targeting moiety and the immunoglobulin chain constant region, or the immune cell engager and the immunoglobulin chain constant region.

In some embodiments, the linker is selected from: a cleavable linker, a non-cleavable linker, a peptide linker, a flexible linker, a rigid linker, a helical linker, or a non-helical linker.

In some embodiments, the linker is a peptide linker.

In some embodiments, the peptide linker comprises Gly and Ser.

In another aspect, provides herein is an isolated nucleic acid molecule, which comprises the nucleotide sequence encoding any one of the antibody molecules or the polypeptide molecule described herein, or a nucleotide sequence substantially homologous thereto (e.g., at least 95% to 99.9% identical thereto).

In another aspect, provides herein is an isolated nucleic acid encoding the antibody molecule as described herein, or the polypeptide molecule of the composition as described herein.

In another aspect, provides herein is a vector, e.g., an expression vector, comprising one or more of the nucleic acid molecules as described herein.

In another aspect, provides herein is a host cell comprising the nucleic acid molecule as described herein, or the vector as described herein.

In another aspect, provides herein is a method of making, e.g., producing, the antibody molecule as described herein or the composition as described herein, comprising culturing the host cell as described herein, under suitable conditions, e.g., conditions suitable for gene expression and/or homo- or heterodimerization.

In another aspect, provides herein is a pharmaceutical composition comprising the antibody molecule as described herein, the composition as described herein, the nucleic acid molecule as described herein, or the host cell as described herein, and a pharmaceutically acceptable carrier, excipient, diluent, or stabilizer.

In another aspect, provides herein is a method of treating a cancer, comprising administering to a subject in need thereof the antibody molecule as described herein, the composition as described herein, or the pharmaceutical composition as described herein, wherein the antibody molecule, the composition, or the pharmaceutical composition is administered in an amount effective to treat the cancer.

In another aspect, provides herein is the antibody molecule as described herein or the composition as described herein for use in treating cancer.

In some embodiments, the cancer is a solid tumor cancer, or a metastatic lesion.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a graph showing binding of NKp30 antibodies to NK92 cells. Data was calculated as the percent-AF747 positive population.

FIG. 2 is a graph showing activation of NK92 cells by NKp30 antibodies. Data were generated using hamster anti-NKp30 mAbs.

### DETAILED DESCRIPTION OF THE INVENTION

### Definitions

Certain terms are defined below.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs).

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full-length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E.A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453 ) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score = 100, wordlength = 12 to obtain nucleotide sequences homologous to a nucleic acid molecule of the invention. BLAST protein searches can be performed with the XBLAST program, score = 50, wordlength = 3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See ncbi.nlm.nih.gov.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L- optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Natural Killer Cell Engagers

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis.

The present disclosure provides, inter alia, antibody molecules, e.g., multispecific (e.g., bi-, tri-, quad- specific) or multifunctional molecules, that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in Tables 7-10. In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in U.S. Pat. No. 6,979,546, U.S. Pat. No. 9,447,185, PCT Application No. WO2015121383A1, PCT Application No. WO2016110468A1, PCT Application No. WO2004056392A1, or U.S. Application Publication No. US20070231322A1, the sequences of which are hereby incorporated by reference. In some embodiments, binding of the NK cell engager, e.g., antigen binding domain that binds to NKp30, to the NK cell activates the NK cell. An antigen binding domain that binds to NKp30 (e.g., NKp30 present, e.g., expressed or displayed, on the surface of an NK cell) may be said to target NKp30, the NK cell, or both.

In some embodiments, the antigen binding domain that binds to NKp30 comprises one or more CDRs (e.g., VHCDR1, VHCDR2, VHCDR3, VLCDR1, VLCDR2, and/or VLCDR3) disclosed in Table 7, Table 18, or Table 8, or a sequence having at least 85%, 90%, 95%, or 99% identity thereto. In some embodiments, the antigen binding domain that binds to NKp30 comprises one or more framework regions (e.g., VHFWR1, VHFWR2, VHFWR3, VHFWR4, VLFWR1, VLFWR2, VLFWR3, and/or VLFWR4) disclosed in Table 7, Table 18, or Table 8, or a sequence having at least 85%, 90%, 95%, or 99% identity thereto. In some embodiments, the antigen binding domain that binds to NKp30 comprises a VH and/or a VL disclosed in Table 9, or a sequence having at least 85%, 90%, 95%, or 99% identity thereto. In some embodiments, any of the VH domains disclosed in Table 9 may be paired with any of the VL domains disclosed in Table 9 to form the antigen binding domain that binds to NKp30. In some embodiments, the antigen binding domain that binds to NKp30 comprises an amino acid sequence disclosed in Table 10, or a sequence having at least 85%, 90%, 95%, or 99% identity thereto.

In some embodiments, the antigen binding domain that binds to NKP30 comprises one or more CDRs (e.g., VHCDR1, VHCDR2, VHCDR3, VLCDR1, VLCDR2, and/or VLCDR3) disclosed in Table 8A and/or 8B, or a sequence having at least 85%, 90%, 95%, or 99% identity thereto. In some embodiments, the antigen binding domain that binds to NKP30 comprises one or more framework regions (e.g., VHFWR1, VHFWR2, VHFWR3, VHFWR4, VLFWR1, VLFWR2, VLFWR3, and/or VLFWR4) disclosed in Table 8A and/or 8B, or a sequence having at least 85%, 90%, 95%, or 99% identity thereto. In some embodiments, the antigen binding domain that binds to NKP30 comprises a VH and/or a VL disclosed in Table 9, or a sequence having at least 85%, 90%, 95%, or 99% identity thereto.

In some embodiments, the antigen binding domain that binds to NKp30 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of SEQ ID NOs: 7313, 6001, and 7315, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 6001, and 6002, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 6008, and 6009, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 7385, and 7315, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 7318, and 6009, respectively (or a sequence having at least 85%, 90%, 95%, or 99% identity thereto).

In some embodiments, the VLCDR1, VLCDR2, and VLCDR3 comprise the amino acid sequences of SEQ ID NOs: 7326, 7327, and 7329, respectively (or a sequence having at least 85%, 90%, 95%, or 99% identity thereto). In some embodiments, the VLCDR1, VLCDR2, and VLCDR3 comprise the amino acid sequences of SEQ ID NOs: 6063, 6064, and 7293, respectively (or a sequence having at least 85%, 90%, 95%, or 99% identity thereto). In some embodiments, the VLCDR1, VLCDR2, and VLCDR3 comprise the amino acid sequences of SEQ ID NOs: 6070, 6071, and 6072, respectively (or a sequence having at least 85%, 90%, 95%, or 99% identity thereto). In some embodiments, the VLCDR1, VLCDR2, and VLCDR3 comprise the amino acid sequences of SEQ ID NOs: 6070, 6064, and 7321, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of SEQ ID NOs: 7313, 6001, 7315, 7326, 7327, and 7329, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6001, 6002, 6063, 6064, and 7293, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6008, 6009, 6070, 6071, and 6072, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7385, 7315, 6070, 6064, and 7321, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7318, 6009, 6070, 6064, and 7321, respectively (or a sequence having at least 85%, 90%, 95%, or 99% identity thereto).

In some embodiments, the VH comprises an amino acid sequence selected from the group consisting of SEQ ID NOs: 7298 or 7300-7304 (or a sequence having at least 85%, 90%, 95%, or 99% identity thereto) and/or the VL comprises an amino acid sequence selected from the group consisting of SEQ ID NOs: 7299 or 7305-7309 (or a sequence having at least 85%, 90%, 95%, or 99% identity thereto). In some embodiments, the VH and VL comprise the amino acid sequences of SEQ ID NOs: 7302 and 7305, respectively (or a sequence having at least 85%, 90%, 95%, or 99% identity thereto). In some embodiments, the VH and VL comprise the amino acid sequences of SEQ ID NOs: 7302 and 7309, respectively (or a sequence having at least 85%, 90%, 95%, or 99% identity thereto).

In some embodiments, the VH comprises an amino acid sequence selected from the group consisting of SEQ ID NOs: 6121 or 6123-6128 (or a sequence having at least 85%, 90%, 95%, or 99% identity thereto) and/or the VL comprises an amino acid sequence selected from the group consisting of SEQ ID NOs: 7294 or 6137-6141 (or a sequence having at least 85%, 90%, 95%, or 99% identity thereto). In some embodiments, the VH comprises an amino acid sequence selected from the group consisting of SEQ ID NOs: 6122 or 6129-6134 (or a sequence having at least 85%, 90%, 95%, or 99% identity thereto) and/or the VL comprises an amino acid sequence selected from the group consisting of SEQ ID NOs: 6136 or 6142-6147 (or a sequence having at least 85%, 90%, 95%, or 99% identity thereto). In some embodiments, the VH and VL comprise the amino acid sequences of SEQ ID NOs: 7295 and 7296, respectively (or a sequence having at least 85%, 90%, 95%, or 99% identity thereto). In some embodiments, the VH and VL comprise the amino acid sequences of SEQ ID NOs: 7297 and 7296, respectively (or a sequence having at least 85%, 90%, 95%, or 99% identity thereto). In some embodiments, the VH and VL comprise the amino acid sequences of SEQ ID NOs: 6122 and 6136, respectively (or a sequence having at least 85%, 90%, 95%, or 99% identity thereto).

In some embodiments, the antigen binding domain that binds to NKp30 comprises the amino acid sequence of SEQ ID NO: 7310 (or a sequence having at least 85%, 90%, 95%, or 99% identity thereto). In some embodiments, the antigen binding domain that binds to NKp30 comprises the amino acid sequence of SEQ ID NO: 7311 (or a sequence having at least 85%, 90%, 95%, or 99% identity thereto). In some embodiments, the antigen binding domain that binds to NKp30 comprises the amino acid sequence of SEQ ID NOs: 17, and 7386-7392 (or a sequence having at least 85%, 90%, 95%, or 99% identity thereto).

In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002.

In some embodiments, the antigen binding domain that targets NKp30 comprises a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293(or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002, and a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009.

In some embodiments, the antigen binding domain that targets NKp30 comprises a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009, and a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 6006.

In some embodiments, the antigen binding domain that targets NKp30 comprises a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 6006, and a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6006.

In some embodiments, the antigen binding domain that targets NKp30 comprises a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6006, and a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013.

In some embodiments, the antigen binding domain that targets NKp30 comprises a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013, and a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013.

In some embodiments, the antigen binding domain that targets NKp30 comprises a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013, and a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6014, a VHFWR2 amino acid sequence of SEQ ID NO: 6015, a VHFWR3 amino acid sequence of SEQ ID NO: 6016, and/or a VHFWR4 amino acid sequence of SEQ ID NO: 6017.

In some embodiments, the antigen binding domain that targets NKp30 comprises a VH comprising a VHFWR1 amino acid sequence of SEQ ID NO: 6014 (or a sequence with no more than 1, 2, 3, 4, 5, or 6 mutations, e.g., substitutions, additions, or deletions, therefrom), a VHFWR2 amino acid sequence of SEQ ID NO: 6015 (or a sequence with no more than 1, 2, 3, 4, 5, or 6 mutations, e.g., substitutions, additions, or deletions, therefrom), a VHFWR3 amino acid sequence of SEQ ID NO: 6016 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6017.

In some embodiments, the antigen binding domain that targets NKp30 comprises a VL comprising a light chain framework region 1 (VLFWR1) amino acid sequence of SEQ ID NO: 6077, a VLFWR2 amino acid sequence of SEQ ID NO: 6078, a VLFWR3 amino acid sequence of SEQ ID NO: 6079, and/or a VLFWR4 amino acid sequence of SEQ ID NO: 6080.

In some embodiments, the antigen binding domain that targets NKp30 comprises a VL comprising a VLFWR1 amino acid sequence of SEQ ID NO: 6077 (or a sequence with no more than 1, 2, or 3 mutations, e.g., substitutions, additions, or deletions), a VLFWR2 amino acid sequence of SEQ ID NO: 6078 (or a sequence with no more than 1 mutation, e.g., substitution, addition, or deletion), a VLFWR3 amino acid sequence of SEQ ID NO: 6079 (or a sequence with no more than 1 mutation, e.g., substitution, addition, or deletion), and/or a VLFWR4 amino acid sequence of SEQ ID NO: 6080.

In some embodiments, the antigen binding domain that targets NKp30 comprises a VH comprising a heavy chain framework region 1 (VHFWR1) amino acid sequence of SEQ ID NO: 6018, a VHFWR2 amino acid sequence of SEQ ID NO: 6019, a VHFWR3 amino acid sequence of SEQ ID NO: 6020, and/or a VHFWR4 amino acid sequence of SEQ ID NO: 6021.

In some embodiments, the antigen binding domain that targets NKp30 comprises a VH comprising a VHFWR1 amino acid sequence of SEQ ID NO: 6018 (or a sequence with no more than 1, 2, 3, 4, 5, or 6 mutations, e.g., substitutions, additions, or deletions, therefrom), a VHFWR2 amino acid sequence of SEQ ID NO: 6019 (or a sequence with no more than 1, 2, 3, 4, 5, or 6 mutations, e.g., substitutions, additions, or deletions, therefrom), a VHFWR3 amino acid sequence of SEQ ID NO: 6020 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6021.

In some embodiments, the antigen binding domain that targets NKp30 comprises a VL comprising a light chain framework region 1 (VLFWR1) amino acid sequence of SEQ ID NO: 6081, a VLFWR2 amino acid sequence of SEQ ID NO: 6082, a VLFWR3 amino acid sequence of SEQ ID NO: 6083, and/or a VLFWR4 amino acid sequence of SEQ ID NO: 6084.

In some embodiments, the antigen binding domain that targets NKp30 comprises a VL comprising a VLFWR1 amino acid sequence of SEQ ID NO: 6081 (or a sequence with no more than 1, 2, or 3 mutations, e.g., substitutions, additions, or deletions), a VLFWR2 amino acid sequence of SEQ ID NO: 6082 (or a sequence with no more than 1 mutation, e.g., substitution, addition, or deletion), a VLFWR3 amino acid sequence of SEQ ID NO: 6083 (or a sequence with no more than 1 mutation, e.g., substitution, addition, or deletion), and/or a VLFWR4 amino acid sequence of SEQ ID NO: 6084.

In some embodiments, the antigen binding domain that targets NKp30 comprises a VH comprising a heavy chain framework region 1 (VHFWR1) amino acid sequence of SEQ ID NO: 6022, a VHFWR2 amino acid sequence of SEQ ID NO: 6023, a VHFWR3 amino acid sequence of SEQ ID NO: 6024, and/or a VHFWR4 amino acid sequence of SEQ ID NO: 6025.

In some embodiments, the antigen binding domain that targets NKp30 comprises a VH comprising a VHFWR1 amino acid sequence of SEQ ID NO: 6022 (or a sequence with no more than 1, 2, 3, 4, 5, or 6 mutations, e.g., substitutions, additions, or deletions, therefrom), a VHFWR2 amino acid sequence of SEQ ID NO: 6023 (or a sequence with no more than 1, 2, 3, 4, 5, or 6 mutations, e.g., substitutions, additions, or deletions, therefrom), a VHFWR3 amino acid sequence of SEQ ID NO: 6024 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6025.

In some embodiments, the antigen binding domain that targets NKp30 comprises a VL comprising a light chain framework region 1 (VLFWR1) amino acid sequence of SEQ ID NO: 6085, a VLFWR2 amino acid sequence of SEQ ID NO: 6086, a VLFWR3 amino acid sequence of SEQ ID NO: 6087, and/or a VLFWR4 amino acid sequence of SEQ ID NO: 6088.

In some embodiments, the antigen binding domain that targets NKp30 comprises a VL comprising a VLFWR1 amino acid sequence of SEQ ID NO: 6085 (or a sequence with no more than 1, 2, or 3 mutations, e.g., substitutions, additions, or deletions), a VLFWR2 amino acid sequence of SEQ ID NO: 6086 (or a sequence with no more than 1 mutation, e.g., substitution, addition, or deletion), a VLFWR3 amino acid sequence of SEQ ID NO: 6087 (or a sequence with no more than 1 mutation, e.g., substitution, addition, or deletion), and/or a VLFWR4 amino acid sequence of SEQ ID NO: 6088.

In some embodiments, the antigen binding domain that targets NKp30 comprises a VH comprising a heavy chain framework region 1 (VHFWR1) amino acid sequence of SEQ ID NO: 6026, a VHFWR2 amino acid sequence of SEQ ID NO: 6027, a VHFWR3 amino acid sequence of SEQ ID NO: 6028, and/or a VHFWR4 amino acid sequence of SEQ ID NO: 6029.

In some embodiments, the antigen binding domain that targets NKp30 comprises a VH comprising a VHFWR1 amino acid sequence of SEQ ID NO: 6026 (or a sequence with no more than 1, 2, 3, 4, 5, or 6 mutations, e.g., substitutions, additions, or deletions, therefrom), a VHFWR2 amino acid sequence of SEQ ID NO: 6027 (or a sequence with no more than 1, 2, 3, 4, 5, or 6 mutations, e.g., substitutions, additions, or deletions, therefrom), a VHFWR3 amino acid sequence of SEQ ID NO: 6028 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6029.

In some embodiments, the antigen binding domain that targets NKp30 comprises a VL comprising a light chain framework region 1 (VLFWR1) amino acid sequence of SEQ ID NO: 6089, a VLFWR2 amino acid sequence of SEQ ID NO: 6090, a VLFWR3 amino acid sequence of SEQ ID NO: 6091, and/or a VLFWR4 amino acid sequence of SEQ ID NO: 6092.

In some embodiments, the antigen binding domain that targets NKp30 comprises a VL comprising a VLFWR1 amino acid sequence of SEQ ID NO: 6089 (or a sequence with no more than 1, 2, or 3 mutations, e.g., substitutions, additions, or deletions), a VLFWR2 amino acid sequence of SEQ ID NO: 6090 (or a sequence with no more than 1 mutation, e.g., substitution, addition, or deletion), a VLFWR3 amino acid sequence of SEQ ID NO: 6091 (or a sequence with no more than 1 mutation, e.g., substitution, addition, or deletion), and/or a VLFWR4 amino acid sequence of SEQ ID NO: 6092.

In some embodiments, the antigen binding domain that targets NKp30 comprises a VH comprising a heavy chain framework region 1 (VHFWR1) amino acid sequence of SEQ ID NO: 6030, a VHFWR2 amino acid sequence of SEQ ID NO: 6032, a VHFWR3 amino acid sequence of SEQ ID NO: 6033, and/or a VHFWR4 amino acid sequence of SEQ ID NO: 6034.

In some embodiments, the antigen binding domain that targets NKp30 comprises a VH comprising a VHFWR1 amino acid sequence of SEQ ID NO: 6030 (or a sequence with no more than 1, 2, 3, 4, 5, or 6 mutations, e.g., substitutions, additions, or deletions, therefrom), a VHFWR2 amino acid sequence of SEQ ID NO: 6032 (or a sequence with no more than 1, 2, 3, 4, 5, or 6 mutations, e.g., substitutions, additions, or deletions, therefrom), a VHFWR3 amino acid sequence of SEQ ID NO: 6033 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6034.

In some embodiments, the antigen binding domain that targets NKp30 comprises a VL comprising a light chain framework region 1 (VLFWR1) amino acid sequence of SEQ ID NO: 6093, a VLFWR2 amino acid sequence of SEQ ID NO: 6094, a VLFWR3 amino acid sequence of SEQ ID NO: 6095, and/or a VLFWR4 amino acid sequence of SEQ ID NO: 6096.

In some embodiments, the antigen binding domain that targets NKp30 comprises a VL comprising a VLFWR1 amino acid sequence of SEQ ID NO: 6093 (or a sequence with no more than 1, 2, or 3 mutations, e.g., substitutions, additions, or deletions), a VLFWR2 amino acid sequence of SEQ ID NO: 6094 (or a sequence with no more than 1 mutation, e.g., substitution, addition, or deletion), a VLFWR3 amino acid sequence of SEQ ID NO: 6095 (or a sequence with no more than 1 mutation, e.g., substitution, addition, or deletion), and/or a VLFWR4 amino acid sequence of SEQ ID NO: 6096.

In some embodiments, the antigen binding domain that targets NKp30 comprises a VH comprising a heavy chain framework region 1 (VHFWR1) amino acid sequence of SEQ ID NO: 6035, a VHFWR2 amino acid sequence of SEQ ID NO: 6036, a VHFWR3 amino acid sequence of SEQ ID NO: 6037, and/or a VHFWR4 amino acid sequence of SEQ ID NO: 6038.

In some embodiments, the antigen binding domain that targets NKp30 comprises a VH comprising a VHFWR1 amino acid sequence of SEQ ID NO: 6035 (or a sequence with no more than 1, 2, 3, 4, 5, or 6 mutations, e.g., substitutions, additions, or deletions, therefrom), a VHFWR2 amino acid sequence of SEQ ID NO: 6036 (or a sequence with no more than 1, 2, 3, 4, 5, or 6 mutations, e.g., substitutions, additions, or deletions, therefrom), a VHFWR3 amino acid sequence of SEQ ID NO: 6037 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6038.

In some embodiments, the antigen binding domain that targets NKp30 comprises a VH comprising a heavy chain framework region 1 (VHFWR1) amino acid sequence of SEQ ID NO: 6039, a VHFWR2 amino acid sequence of SEQ ID NO: 6040, a VHFWR3 amino acid sequence of SEQ ID NO: 6041, and/or a VHFWR4 amino acid sequence of SEQ ID NO: 6042.

In some embodiments, the antigen binding domain that targets NKp30 comprises a VH comprising a VHFWR1 amino acid sequence of SEQ ID NO: 6039 (or a sequence with no more than 1, 2, 3, 4, 5, or 6 mutations, e.g., substitutions, additions, or deletions, therefrom), a VHFWR2 amino acid sequence of SEQ ID NO: 6040 (or a sequence with no more than 1, 2, 3, 4, 5, or 6 mutations, e.g., substitutions, additions, or deletions, therefrom), a VHFWR3 amino acid sequence of SEQ ID NO: 6041 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6042.

In some embodiments, the antigen binding domain that targets NKp30 comprises a VL comprising a light chain framework region 1 (VLFWR1) amino acid sequence of SEQ ID NO: 6097, a VLFWR2 amino acid sequence of SEQ ID NO: 6098, a VLFWR3 amino acid sequence of SEQ ID NO: 6099, and/or a VLFWR4 amino acid sequence of SEQ ID NO: 6100.

In some embodiments, the antigen binding domain that targets NKp30 comprises a VL comprising a VLFWR1 amino acid sequence of SEQ ID NO: 6097 (or a sequence with no more than 1, 2, or 3 mutations, e.g., substitutions, additions, or deletions), a VLFWR2 amino acid sequence of SEQ ID NO: 6098 (or a sequence with no more than 1 mutation, e.g., substitution, addition, or deletion), a VLFWR3 amino acid sequence of SEQ ID NO: 6099 (or a sequence with no more than 1 mutation, e.g., substitution, addition, or deletion), and/or a VLFWR4 amino acid sequence of SEQ ID NO: 6100.

In some embodiments, the antigen binding domain that targets NKp30 comprises a VH comprising a heavy chain framework region 1 (VHFWR1) amino acid sequence of SEQ ID NO: 6043, a VHFWR2 amino acid sequence of SEQ ID NO: 6044, a VHFWR3 amino acid sequence of SEQ ID NO: 6045, and/or a VHFWR4 amino acid sequence of SEQ ID NO: 6046.

In some embodiments, the antigen binding domain that targets NKp30 comprises a VH comprising a VHFWR1 amino acid sequence of SEQ ID NO: 6043 (or a sequence with no more than 1, 2, 3, 4, 5, or 6 mutations, e.g., substitutions, additions, or deletions, therefrom), a VHFWR2 amino acid sequence of SEQ ID NO: 6044 (or a sequence with no more than 1, 2, 3, 4, 5, or 6 mutations, e.g., substitutions, additions, or deletions, therefrom), a VHFWR3 amino acid sequence of SEQ ID NO: 6045 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6046.

In some embodiments, the antigen binding domain that targets NKp30 comprises a VL comprising a light chain framework region 1 (VLFWR1) amino acid sequence of SEQ ID NO: 6101, a VLFWR2 amino acid sequence of SEQ ID NO: 6102, a VLFWR3 amino acid sequence of SEQ ID NO: 6103, and/or a VLFWR4 amino acid sequence of SEQ ID NO: 6104.

In some embodiments, the antigen binding domain that targets NKp30 comprises a VL comprising a VLFWR1 amino acid sequence of SEQ ID NO: 6101 (or a sequence with no more than 1, 2, or 3 mutations, e.g., substitutions, additions, or deletions), a VLFWR2 amino acid sequence of SEQ ID NO: 6102 (or a sequence with no more than 1 mutation, e.g., substitution, addition, or deletion), a VLFWR3 amino acid sequence of SEQ ID NO: 6103 (or a sequence with no more than 1 mutation, e.g., substitution, addition, or deletion), and/or a VLFWR4 amino acid sequence of SEQ ID NO: 6104.

In some embodiments, the antigen binding domain that targets NKp30 comprises a VH comprising a heavy chain framework region 1 (VHFWR1) amino acid sequence of SEQ ID NO: 6047, a VHFWR2 amino acid sequence of SEQ ID NO: 6048, a VHFWR3 amino acid sequence of SEQ ID NO: 6049, and/or a VHFWR4 amino acid sequence of SEQ ID NO: 6050.

In some embodiments, the antigen binding domain that targets NKp30 comprises a VH comprising a VHFWR1 amino acid sequence of SEQ ID NO: 6047 (or a sequence with no more than 1, 2, 3, 4, 5, or 6 mutations, e.g., substitutions, additions, or deletions, therefrom), a VHFWR2 amino acid sequence of SEQ ID NO: 6048 (or a sequence with no more than 1, 2, 3, 4, 5, or 6 mutations, e.g., substitutions, additions, or deletions, therefrom), a VHFWR3 amino acid sequence of SEQ ID NO: 6049 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6050.

In some embodiments, the antigen binding domain that targets NKp30 comprises a VL comprising a light chain framework region 1 (VLFWR1) amino acid sequence of SEQ ID NO: 6105, a VLFWR2 amino acid sequence of SEQ ID NO: 6106, a VLFWR3 amino acid sequence of SEQ ID NO: 6107, and/or a VLFWR4 amino acid sequence of SEQ ID NO: 6108.

In some embodiments, the antigen binding domain that targets NKp30 comprises a VL comprising a VLFWR1 amino acid sequence of SEQ ID NO: 6105 (or a sequence with no more than 1, 2, or 3 mutations, e.g., substitutions, additions, or deletions), a VLFWR2 amino acid sequence of SEQ ID NO: 6106 (or a sequence with no more than 1 mutation, e.g., substitution, addition, or deletion), a VLFWR3 amino acid sequence of SEQ ID NO: 6107 (or a sequence with no more than 1 mutation, e.g., substitution, addition, or deletion), and/or a VLFWR4 amino acid sequence of SEQ ID NO: 6108.

In some embodiments, the antigen binding domain that targets NKp30 comprises a VH comprising a heavy chain framework region 1 (VHFWR1) amino acid sequence of SEQ ID NO: 6051, a VHFWR2 amino acid sequence of SEQ ID NO: 6052, a VHFWR3 amino acid sequence of SEQ ID NO: 6053, and/or a VHFWR4 amino acid sequence of SEQ ID NO: 6054.

In some embodiments, the antigen binding domain that targets NKp30 comprises a VH comprising a VHFWR1 amino acid sequence of SEQ ID NO: 6051 (or a sequence with no more than 1, 2, 3, 4, 5, or 6 mutations, e.g., substitutions, additions, or deletions, therefrom), a VHFWR2 amino acid sequence of SEQ ID NO: 6052 (or a sequence with no more than 1, 2, 3, 4, 5, or 6 mutations, e.g., substitutions, additions, or deletions, therefrom), a VHFWR3 amino acid sequence of SEQ ID NO: 6053 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6054.

In some embodiments, the antigen binding domain that targets NKp30 comprises a VL comprising a light chain framework region 1 (VLFWR1) amino acid sequence of SEQ ID NO: 6109, a VLFWR2 amino acid sequence of SEQ ID NO: 6110, a VLFWR3 amino acid sequence of SEQ ID NO: 6111, and/or a VLFWR4 amino acid sequence of SEQ ID NO: 6112.

In some embodiments, the antigen binding domain that targets NKp30 comprises a VL comprising a VLFWR1 amino acid sequence of SEQ ID NO: 6109 (or a sequence with no more than 1, 2, or 3 mutations, e.g., substitutions, additions, or deletions), a VLFWR2 amino acid sequence of SEQ ID NO: 6110 (or a sequence with no more than 1 mutation, e.g., substitution, addition, or deletion), a VLFWR3 amino acid sequence of SEQ ID NO: 6111 (or a sequence with no more than 1 mutation, e.g., substitution, addition, or deletion), and/or a VLFWR4 amino acid sequence of SEQ ID NO: 6112.

In some embodiments, the antigen binding domain that targets NKp30 comprises a VH comprising a heavy chain framework region 1 (VHFWR1) amino acid sequence of SEQ ID NO: 6055, a VHFWR2 amino acid sequence of SEQ ID NO: 6056, a VHFWR3 amino acid sequence of SEQ ID NO: 6057, and/or a VHFWR4 amino acid sequence of SEQ ID NO: 6058.

In some embodiments, the antigen binding domain that targets NKp30 comprises a VH comprising a VHFWR1 amino acid sequence of SEQ ID NO: 6055 (or a sequence with no more than 1, 2, 3, 4, 5, or 6 mutations, e.g., substitutions, additions, or deletions, therefrom), a VHFWR2 amino acid sequence of SEQ ID NO: 6056 (or a sequence with no more than 1, 2, 3, 4, 5, or 6 mutations, e.g., substitutions, additions, or deletions, therefrom), a VHFWR3 amino acid sequence of SEQ ID NO: 6057 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6058.

In some embodiments, the antigen binding domain that targets NKp30 comprises a VL comprising a light chain framework region 1 (VLFWR1) amino acid sequence of SEQ ID NO: 6113, a VLFWR2 amino acid sequence of SEQ ID NO: 6114, a VLFWR3 amino acid sequence of SEQ ID NO: 6115, and/or a VLFWR4 amino acid sequence of SEQ ID NO: 6116.

In some embodiments, the antigen binding domain that targets NKp30 comprises a VL comprising a VLFWR1 amino acid sequence of SEQ ID NO: 6113 (or a sequence with no more than 1, 2, or 3 mutations, e.g., substitutions, additions, or deletions), a VLFWR2 amino acid sequence of SEQ ID NO: 6114 (or a sequence with no more than 1 mutation, e.g., substitution, addition, or deletion), a VLFWR3 amino acid sequence of SEQ ID NO: 6115 (or a sequence with no more than 1 mutation, e.g., substitution, addition, or deletion), and/or a VLFWR4 amino acid sequence of SEQ ID NO: 6116.

In some embodiments, the antigen binding domain that targets NKp30 comprises a VH comprising a heavy chain framework region 1 (VHFWR1) amino acid sequence of SEQ ID NO: 6059, a VHFWR2 amino acid sequence of SEQ ID NO: 6060, a VHFWR3 amino acid sequence of SEQ ID NO: 6061, and/or a VHFWR4 amino acid sequence of SEQ ID NO: 6062.

In some embodiments, the antigen binding domain that targets NKp30 comprises a VH comprising a VHFWR1 amino acid sequence of SEQ ID NO: 6059 (or a sequence with no more than 1, 2, 3, 4, 5, or 6 mutations, e.g., substitutions, additions, or deletions, therefrom), a VHFWR2 amino acid sequence of SEQ ID NO: 6060 (or a sequence with no more than 1, 2, 3, 4, 5, or 6 mutations, e.g., substitutions, additions, or deletions, therefrom), a VHFWR3 amino acid sequence of SEQ ID NO: 6061 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6062.

In some embodiments, the antigen binding domain that targets NKp30 comprises a VL comprising a light chain framework region 1 (VLFWR1) amino acid sequence of SEQ ID NO: 6117, a VLFWR2 amino acid sequence of SEQ ID NO: 6118, a VLFWR3 amino acid sequence of SEQ ID NO: 6119, and/or a VLFWR4 amino acid sequence of SEQ ID NO: 6120.

In some embodiments, the antigen binding domain that targets NKp30 comprises a VL comprising a VLFWR1 amino acid sequence of SEQ ID NO: 6117 (or a sequence with no more than 1, 2, or 3 mutations, e.g., substitutions, additions, or deletions), a VLFWR2 amino acid sequence of SEQ ID NO: 6118 (or a sequence with no more than 1 mutation, e.g., substitution, addition, or deletion), a VLFWR3 amino acid sequence of SEQ ID NO: 6119 (or a sequence with no more than 1 mutation, e.g., substitution, addition, or deletion), and/or a VLFWR4 amino acid sequence of SEQ ID NO: 6120.

In some embodiments, the antigen binding domain that targets NKp30 comprises a VH comprising the amino acid sequence of SEQ ID NO: 6148 (or an amino acid sequence having at least about 77%, 80%, 85%, 90%, 95%, or 99% sequence identity to SEQ ID NO: 6148). In some embodiments, the antigen binding domain that targets NKp30 comprises a VH comprising the amino acid sequence of SEQ ID NO: 6149 (or an amino acid sequence having at least about 77%, 80%, 85%, 90%, 95%, or 99% sequence identity to SEQ ID NO: 6149). In some embodiments, the antigen binding domain that targets NKp30 comprises a VL comprising the amino acid sequence of SEQ ID NO: 6150 (or an amino acid sequence having at least about 93%, 95%, or 99% sequence identity to SEQ ID NO: 6150). In some embodiments, antigen binding domain that targets NKp30 comprises a VH comprising the amino acid sequence of SEQ ID NO: 6148. In some embodiments, antigen binding domain that targets NKp30 comprises a VH comprising the amino acid sequence of SEQ ID NO: 6149. In some embodiments, the antigen binding domain that targets NKp30 comprises a VL comprising the amino acid sequence of SEQ ID NO: 6150.

In some embodiments, the antigen binding domain that targets NKp30 comprises a VH comprising the amino acid sequence of SEQ ID NO: 6148, and a VL comprising the amino acid sequence of SEQ ID NO: 6150. In some embodiments, the antigen binding domain that targets NKp30 comprises a VH comprising the amino acid sequence of SEQ ID NO: 6149, and a VL comprising the amino acid sequence of SEQ ID NO: 6150.

In some embodiments, the antigen binding domain that targets NKp30 comprises a VH comprising the amino acid sequence of SEQ ID NO: 6151 (or an amino acid sequence having at least about 77%, 80%, 85%, 90%, 95%, or 99% sequence identity to SEQ ID NO: 6151). In some embodiments, the antigen binding domain that targets NKp30 comprises a VH comprising the amino acid sequence of SEQ ID NO: 6152 (or an amino acid sequence having at least about 77%, 80%, 85%, 90%, 95%, or 99% sequence identity to SEQ ID NO: 6152). In some embodiments, the antigen binding domain that targets NKp30 comprises a VL comprising the amino acid sequence of SEQ ID NO: 6153 (or an amino acid sequence having at least about 93%, 95%, or 99% sequence identity to SEQ ID NO: 6153). In some embodiments, antigen binding domain that targets NKp30 comprises a VH comprising the amino acid sequence of SEQ ID NO: 6151. In some embodiments, antigen binding domain that targets NKp30 comprises a VH comprising the amino acid sequence of SEQ ID NO: 6152. In some embodiments, the antigen binding domain that targets NKp30 comprises a VL comprising the amino acid sequence of SEQ ID NO: 6153.

In some embodiments, the antigen binding domain that targets NKp30 comprises a VH comprising the amino acid sequence of SEQ ID NO: 6151, and a VL comprising the amino acid sequence of SEQ ID NO: 6153. In some embodiments, the antigen binding domain that targets NKp30 comprises a VH comprising the amino acid sequence of SEQ ID NO: 6152, and a VL comprising the amino acid sequence of SEQ ID NO: 6153.

In some embodiments, the antigen binding domain that targets NKp30 comprises an scFv. In some embodiments, the scFv comprises an amino acid sequence selected from SEQ ID NOs: 17, and 7386-7392, or an amino acid sequence having at least about 93%, 95%, or 99% sequence identity thereto.

TABLE 7

Exemplary heavy chain CDRs and FWRs of NKp30-targeting antigen binding domains

| | | | | | | | |
|---|---|---|---|---|---|---|---|
| Ab ID | VHFWR1 | VHCDR1 | VHFWR2 | VHCDR2 | VHFWR3 | VHCDR3 | VHFWR4 |
| 9G1-HC | QIQLQES GPGLVK PSQSLSL TCSVTGF SIN (SEQ ID NO: 6003) | TGGYHW N (SEQ ID NO: 6000) | WIRQFP GKKLEW MG (SEQ ID NO: 6004) | YIYSSGS TSYNPSL KS (SEQ ID NO: 6001) | RISITRD TSKNQFF LQLNSV TTEDTA TYYCAR (SEQ ID NO: 6005) | GNWHYF DF (SEQ ID NO: 6002) | WGQGT MVTVSS (SEQ ID NO: 6006) |
| 15H6-HC | QIQLQES GPGLVK PSQSLSL TCSVTGF SIN (SEQ ID NO: 6010) | TGGYHW N (SEQ ID NO: 6007) | WIRQFP GKKLEW MG (SEQ ID NO: 6011) | YIYSSGT TRYNPSL KS (SEQ ID NO: 6008) | RISITRD TSKNQFF LQLNSV TPEDTAT YYCTR (SEQ ID NO: 6012) | GNWHYF DY (SEQ ID NO: 6009) | WGQGTL VAVSS (SEQ ID NO: 6013) |
| 9G1-HC_1 | QIQLQES GPGLVK PSETLSL TCTVSGF SIN (SEQ ID NO: 6014) | TGGYHW N (SEQ ID NO: 6000) | WIRQPA GKGLEW IG (SEQ ID NO: 6015) | YIYSSGS TSYNPSL KS (SEQ ID NO: 6001) | RVTMSR DTSKNQ FSLKLSS VTAADT AVYYCA R (SEQ ID NO: 6016) | GNWHYF DF (SEQ ID NO: 6002) | WGQGT MVTVSS (SEQ ID NO: 6017) |
| 9G1-HC_2 | QIQLQES GPGLVK PSQTLSL TCTVSGF SIN (SEQ ID NO: 6018) | TGGYHW N (SEQ ID NO: 6000) | WIRQHP GKGLEW IG (SEQ ID NO: 6019) | YIYSSGS TSYNPSL KS (SEQ ID NO: 6001) | LVTISRD TSKNQFS LKLSSVT AADTAV YYCAR (SEQ ID NO: 6020) | GNWHYF DF (SEQ ID NO: 6002) | WGQGT MVTVSS (SEQ ID NO: 6021) |
| 9G1-HC_3 | EIQLLES GGGLVQ PGGSLRL SCAVSG FSIN (SEQ ID NO: 6022) | TGGYHW N (SEQ ID NO: 6000) | WVRQAP GKGLEW VG (SEQ ID NO: 6023) | YIYSSGS TSYNPSL KS (SEQ ID NO: 6001) | RFTISRD TSKNTF YLQMNS LRAEDT AVYYCA R (SEQ ID NO: 6024) | GNWHYF DF (SEQ ID NO: 6002) | WGQGT MVTVSS (SEQ ID NO: 6025) |
| 9G1-HC_4 | QIQLVQS GAEVKK PGSSVK VSCKVS GFSIN (SEQ ID NO: 6026) | TGGYHW N (SEQ ID NO: 6000) | WVRQAP GQGLEW MG (SEQ ID NO: 6027) | YIYSSGS TSYNPSL KS (SEQ ID NO: 6001) | RVTITRD TSTNTFY MELSSL RSEDTA VYYCAR (SEQ ID NO: 6028) | GNWHYF DF (SEQ ID NO: 6002) | WGQGT MVTVSS (SEQ ID NO: 6029) |
| 9G1-HC_5 | EIQLVES GGGLVQ PGGSLRL SCAVSG FSIN (SEQ ID NO: 6030) | TGGYHW N (SEQ ID NO: 6000) | WVRQAP GKGLEW VG (SEQ ID NOL 6032) | YIYSSGS TSYNPSL KS (SEQ ID NO: 6001) | RFTISRD TAKNSF YLQMNS LRAEDT AVYYCA R (SEQ ID NO: 6033) | GNWHYF DF (SEQ ID NO: 6002) | WGQGT MVTVSS (SEQ ID NO: 6034) |
| 9G1-HC_6 | QIQLVQS GAEVKK PGASVK VSCKVS GFSIN (SEQ ID NO: 6035) | TGGYHW N (SEQ ID NO: 6000) | WVRQAP GQGLEW MG (SEQ ID NO: 6036) | YIYSSGS TSYNPSL KS (SEQ ID NO: 6001) | RVTMTR DTSTNTF YMELSS LRSEDT AVYYCA R (SEQ ID NO: 6037) | GNWHYF DF (SEQ ID NO: 6002) | WGQGT MVTVSS (SEQ ID NO: 6038) |
| 15H6-HC_1 | QIQLQES GPGLVK PSQTLSL TCTVSGF | TGGYHW N (SEQ ID NO: 6007) | WIRQHP GKGLEW IG (SEQ | YIYSSGT TRYNPSL KS (SEQ | LVTISRD TSKNQFS LKLSSVT AADTAV | GNWHYF DY (SEQ ID NO: 6009) | WGQGTL VTVSS (SEQ ID NO: 6042) |
| | SIN (SEQ ID NO: 6039) | | ID NO: 6040) | ID NO: 6008) | YYCAR (SEQ ID NO: 6041) | | |
| 15H6-HC_2 | QIQLQES GPGLVK PSETLSL TCTVSGF SIN (SEQ ID NO: 6043) | TGGYHW N (SEQ ID NO: 6007) | WIRQPA GKGLEW IG (SEQ ID NO: 6044) | YIYSSGT TRYNPSL KS (SEQ ID NO: 6008) | RVTMSR DTSKNQ FSLKLSS VTAADT AVYYCA R (SEQ ID NO: 6045) | GNWHYF DY (SEQ ID NO: 6009) | WGQGTL VTVSS (SEQ ID NO: 6046) |
| 15H6-HC_3 | EIQLLES GGGLVQ PGGSLRL SCAVSG FSIN (SEQ ID NO: 6047) | TGGYHW N (SEQ ID NO: 6007) | WVRQAP GKGLEW VG (SEQ ID NO: 6048) | YIYSSGT TRYNPSL KS (SEQ ID NO: 6008) | RFTISRD TSKNTF YLQMNS LRAEDT AVYYCA R (SEQ ID NO: 6049) | GNWHYF DY (SEQ ID NO: 6009) | WGQGTL VTVSS (SEQ ID NO: 6050) |
| 15H6-HC_4 | QIQLVES GGGLVK PGGSLRL SCAVSG FSIN (SEQ ID NO: 6051) | TGGYHW N (SEQ ID NO: 6007) | WIRQAP GKGLEW VG (SEQ ID NO: 6052) | YIYSSGT TRYNPSL KS (SEQ ID NO: 6008) | RFTISRD TAKNSF YLQMNS LRAEDT AVYYCA R (SEQ ID NO: 6053) | GNWHYF DY (SEQ ID NO: 6009) | WGQGTL VTVSS (SEQ ID NO: 6054) |
| 15H6-HC_5 | QIQLVQS GAEVKK PGASVK VSCKVS GFSIN (SEQ ID NO: 6055) | TGGYHW N (SEQ ID NO: 6007) | WVRQAP GQGLEW MG (SEQ ID NO: 6056) | YIYSSGT TRYNPSL KS (SEQ ID NO: 6008) | RVTMTR DTSTNTF YMELSS LRSEDT AVYYCA R (SEQ ID NO: 6057) | GNWHYF DY (SEQ ID NO: 6009) | WGQGTL VTVSS (SEQ ID NO: 6058) |
| 15H6-HC_6 | EIQLVQS GAEVKK PGATVKI SCKVSG FSIN (SEQ ID NO: 6059) | TGGYHW N (SEQ ID NO: 6007) | WVQQAP GKGLEW MG (SEQ ID NO: 6060) | YIYSSGT TRYNPSL KS (SEQ ID NO: 6008) | RVTITRD TSTNTFY MELSSL RSEDTA VYYCAR (SEQ ID NO: 6061) | GNWHYF DY (SEQ ID NO: 6009) | WGQGTL VTVSS (SEQ ID NO: 6062) |

TABLE 18

Exemplary heavy chain CDRs and FWRs of NKp30-targeting antigen binding domains (according to the Kabat numbering scheme)

| | | | | | | | |
|---|---|---|---|---|---|---|---|
| Ab ID | VHFWR1 | VHCDR1 | VHFWR2 | VHCDR2 | VHFWR3 | VHCDR3 | VHFWR4 |
| 9G1-HC | QIQLQES GPGLVK PSQSLSL TCSVTGF SINTG (SEQ ID NO: 7317) | GYHWN (SEQ ID NO: 7313) | WIRQFP GKKLEW MG (SEQ ID NO: 6004) | YIYSSGS TSYNPSL KS (SEQ ID NO: 6001) | RISITRD TSKNQFF LQLNSV TTEDTA TYYCAR (SEQ ID NO: 6005) | GNWHYF DF (SEQ ID NO: 6002) | WGQGT MVTVSS (SEQ ID NO: 6006) |
| 15H6-HC | QIQLQES GPGLVK PSQSLSL TCSVTGF SINTG (SEQ ID NO: 7317) | GYHWN (SEQ ID NO: 7313) | WIRQFP GKKLEW MG (SEQ ID NO: 6011) | YIYSSGT TRYNPSL KS (SEQ ID NO: 6008) | RISITRD TSKNQFF LQLNSV TPEDTAT YYCTR (SEQ ID NO: 6012) | GNWHYF DY (SEQ ID NO: 6009) | WGQGTL VAVSS (SEQ ID NO: 6013) |
| 9G1-HC_1 | QIQLQES GPGLVK PSETLSL TCTVSGF SINTG (SEQ ID NO: 7371) | GYHWN (SEQ ID NO: 7313) | WIRQPA GKGLEW IG (SEQ ID NO: 6015) | YIYSSGS TSYNPSL KS (SEQ ID NO: 6001) | RVTMSR DTSKNQ FSLKLSS VTAADT AVYYCA R (SEQ ID NO: 6016) | GNWHYF DF (SEQ ID NO: 6002) | WGQGT MVTVSS (SEQ ID NO: 6017) |
| 9G1-HC_2 | QIQLQES GPGLVK PSQTLSL TCTVSGF SINTG (SEQ ID NO: 7372) | GYHWN (SEQ ID NO: 7313) | WIRQHP GKGLEW IG (SEQ ID NO: 6019) | YIYSSGS TSYNPSL KS (SEQ ID NO: 6001) | LVTISRD TSKNQFS LKLSSVT AADTAV YYCAR (SEQ ID NO: 6020) | GNWHYF DF (SEQ ID NO: 6002) | WGQGT MVTVSS (SEQ ID NO: 6021) |
| 9G1-HC_3 | EIQLLES GGGLVQ PGGSLRL SCAVSG FSINTG (SEQ ID NO: 7373) | GYHWN (SEQ ID NO: 7313) | WVRQAP GKGLEW VG (SEQ ID NO: 6023) | YIYSSGS TSYNPSL KS (SEQ ID NO: 6001) | RFTISRD TSKNTF YLQMNS LRAEDT AVYYCA R (SEQ ID NO: 6024) | GNWHYF DF (SEQ ID NO: 6002) | WGQGT MVTVSS (SEQ ID NO: 6025) |
| 9G1-HC_4 | QIQLVQS GAEVKK PGSSVK VSCKVS GFSINTG (SEQ ID NO: 7374) | GYHWN (SEQ ID NO: 7313) | WVRQAP GQGLEW MG (SEQ ID NO: 6027) | YIYSSGS TSYNPSL KS (SEQ ID NO: 6001) | RVTITRD TSTNTFY MELSSL RSEDTA VYYCAR (SEQ ID NO: 6028) | GNWHYF DF (SEQ ID NO: 6002) | WGQGT MVTVSS (SEQ ID NO: 6029) |
| 9G1-HC_5 | EIQLVES GGGLVQ PGGSLRL SCAVSG FSINTG (SEQ ID NO: 7375) | GYHWN (SEQ ID NO: 7313) | WVRQAP GKGLEW VG (SEQ ID NOL 6032) | YIYSSGS TSYNPSL KS (SEQ ID NO: 6001) | RFTISRD TAKNSF YLQMNS LRAEDT AVYYCA R (SEQ ID NO: 6033) | GNWHYF DF (SEQ ID NO: 6002) | WGQGT MVTVSS (SEQ ID NO: 6034) |
| 9G1-HC_6 | QIQLVQS GAEVKK PGASVK VSCKVS GFSINTG (SEQ ID NO: 7376) | GYHWN (SEQ ID NO: 7313) | WVRQAP GQGLEW MG (SEQ ID NO: 6036) | YIYSSGS TSYNPSL KS (SEQ ID NO: 6001) | RVTMTR DTSTNTF YMELSS LRSEDT AVYYCA R (SEQ ID NO: 6037) | GNWHYF DF (SEQ ID NO: 6002) | WGQGT MVTVSS (SEQ ID NO: 6038) |
| 15H6-HC_1 | QIQLQES GPGLVK PSQTLSL TCTVSGF | GYHWN (SEQ ID NO: 7313) | WIRQHP GKGLEW IG (SEQ | YIYSSGT TRYNPSL KS (SEQ | LVTISRD TSKNQFS LKLSSVT AADTAV | GNWHYF DY (SEQ ID NO: 6009) | WGQGTL VTVSS (SEQ ID NO: 6042) |
| | SINTG (SEQ ID NO: 7372) | | ID NO: 6040) | ID NO: 6008) | YYCAR (SEQ ID NO: 6041) | | |
| 15H6-HC_2 | QIQLQES GPGLVK PSETLSL TCTVSGF SINTG (SEQ ID NO: 7371) | GYHWN (SEQ ID NO: 7313) | WIRQPA GKGLEW IG (SEQ ID NO: 6044) | YIYSSGT TRYNPSL KS (SEQ ID NO: 6008) | RVTMSR DTSKNQ FSLKLSS VTAADT AVYYCA R (SEQ ID NO: 6045) | GNWHYF DY (SEQ ID NO: 6009) | WGQGTL VTVSS (SEQ ID NO: 6046) |
| 15H6-HC_3 | EIQLLES GGGLVQ PGGSLRL SCAVSG FSINTG (SEQ ID NO: 7373) | GYHWN (SEQ ID NO: 7313) | WVRQAP GKGLEW VG (SEQ ID NO: 6048) | YIYSSGT TRYNPSL KS (SEQ ID NO: 6008) | RFTISRD TSKNTF YLQMNS LRAEDT AVYYCA R (SEQ ID NO: 6049) | GNWHYF DY (SEQ ID NO: 6009) | WGQGTL VTVSS (SEQ ID NO: 6050) |
| 15H6-HC_4 | QIQLVES GGGLVK PGGSLRL SCAVSG FSINTG (SEQ ID NO: 7377) | GYHWN (SEQ ID NO: 7313) | WIRQAP GKGLEW VG (SEQ ID NO: 6052) | YIYSSGT TRYNPSL KS (SEQ ID NO: 6008) | RFTISRD TAKNSF YLQMNS LRAEDT AVYYCA R (SEQ ID NO: 6053) | GNWHYF DY (SEQ ID NO: 6009) | WGQGTL VTVSS (SEQ ID NO: 6054) |
| 15H6-HC_5 | QIQLVQS GAEVKK PGASVK VSCKVS GFSINTG (SEQ ID NO: 7376) | GYHWN (SEQ ID NO: 7313) | WVRQAP GQGLEW MG (SEQ ID NO: 6056) | YIYSSGT TRYNPSL KS (SEQ ID NO: 6008) | RVTMTR DTSTNTF YMELSS LRSEDT AVYYCA R (SEQ ID NO: 6057) | GNWHYF DY (SEQ ID NO: 6009) | WGQGTL VTVSS (SEQ ID NO: 6058) |
| 15H6-HC_6 | EIQLVQS GAEVKK PGATVKI SCKVSG FSINTG (SEQ ID NO: 7378) | GYHWN (SEQ ID NO: 7313) | WVQQAP GKGLEW MG (SEQ ID NO: 6060) | YIYSSGT TRYNPSL KS (SEQ ID NO: 6008) | RVTITRD TSTNTFY MELSSL RSEDTA VYYCAR (SEQ ID NO: 6061) | GNWHYF DY (SEQ ID NO: 6009) | WGQGTL VTVSS (SEQ ID NO: 6062) |
| 9D9-HC | QIQLQES GPGLVK PSQSLSL SCSVTGF SINTG (SEQ ID NO: 7312) | GYHWN (SEQ ID NO: 7313) | WIRQFP GKKVEW MG (SEQ ID NO: 7314) | YIYSSGT TKYNPS LKS (SEQ ID NO: 7385) | RISITRD TSKNQFF LQLNSV TTEDTA TYYCAR (SEQ ID NO: 6005) | GDWHYF DY (SEQ ID NO: 7315) | WGQGT MVAVSS (SEQ ID NO: 7316) |
| 3A12-HC | QIQLQES GPGLVK PSQSLSL TCSVTGF SINTG (SEQ ID NO: 7317) | GYHWN (SEQ ID NO: 7313) | WIRQFP GKKLEW MG (SEQ ID NO: 6004) | YIYSSGS TRYNPSL KS (SEQ ID NO: 7318) | RFSITRD TSKNQFF LQLNSV TTEDTA TYYCTR (SEQ ID NO: 7319) | GNWHYF DY (SEQ ID NO: 6009) | WGQGTL VAVSS (SEQ ID NO: 6013) |
| 12D10-HC | QIQLQES GPGLVK PSQSLSL TCSVTGF SINTG (SEQ ID NO: 7317) | GYHWN (SEQ ID NO: 7313) | WIRQFP GKKLEW MG (SEQ ID NO: 6004) | YIYSSGT TRYNPSL KS (SEQ ID NO: 6008) | RISITRD TSKNQFF LQLNSV TPEDTAT YYCTR (SEQ ID NO: 6012) | GNWHYF DY (SEQ ID NO: 6009) | WGQGTL VAVSS (SEQ ID NO: 6013) |
| 15E1-HC | QIQLQES GPGLVK PSQSLSL SCSVTGF SITTT (SEQ ID NO: 7322) | GYHWN (SEQ ID NO: 7313) | WIRQFP GKKLEW MG (SEQ ID NO: 6004) | YIYSSGS TSYNPSL KS (SEQ ID NO: 6001) | RFSITRD TSKNQFF LQLNSV TTEDTA TYYCAR (SEQ ID NO: 7323) | GDWHYF DY (SEQ ID NO: 7315) | WGPGTM VTVSS (SEQ ID NO: 7324) |
| 15E1_ Humanize d variant_V H1 | QIQLQES GPGLVK PSQTLSL TCTVSGF SITTT (SEQ ID NO: 7330) | GYHWN (SEQ ID NO: 7313) | WIRQHP GKGLEW IG (SEQ ID NO: 6019) | YIYSSGS TSYNPSL KS (SEQ ID NO: 6001) | LVTISRD TSKNQFS LKLSSVT AADTAV YYCAR (SEQ ID NO: 6020) | GDWHYF DY (SEQ ID NO: 7315) | WGQGT MVTVSS (SEQ ID NO: 6006) |
| 15E1_ Humanize d variant_V H2 | QIQLVES GGGLVK PGGSLRL SCAVSG FSITTT (SEQ ID NO: 7331) | GYHWN (SEQ ID NO: 7313) | WIRQAP GKGLEW VG (SEQ ID NO: 6052) | YIYSSGS TSYNPSL KS (SEQ ID NO: 6001) | RFTISRD TAKNSF YLQMNS LRAEDT AVYYCA R (SEQ ID NO: 6033) | GDWHYF DY (SEQ ID NO: 7315) | WGQGT MVTVSS (SEQ ID NO: 6006) |
| 15E1_ Humanize d variant_V H3 | EIQLLES GGGLVQ PGGSLRL SCAVSG FSITTT (SEQ ID NO: 7332) | GYHWN (SEQ ID NO: 7313) | WVRQAP GKGLEW VG (SEQ ID NO: 6023) | YIYSSGS TSYNPSL KS (SEQ ID NO: 6001) | RFTISRD TSKNTF YLQMNS LRAEDT AVYYCA R (SEQ ID NO: 6024) | GDWHYF DY (SEQ ID NO: 7315) | WGQGT MVTVSS (SEQ ID NO: 6006) |
| 15E1_ Humanize d variant_V H4 | EIQLVES GGGLVQ PGGSLRL SCAVSG FSITTT (SEQ ID NO: 7333) | GYHWN (SEQ ID NO: 7313) | WVRQAP GKGLEW VG (SEQ ID NO: 6023) | YIYSSGS TSYNPSL KS (SEQ ID NO: 6001) | RFTISRD TAKNSF YLQMNS LRAEDT AVYYCA R (SEQ ID NO: 6033) | GDWHYF DY (SEQ ID NO: 7315) | WGQGT MVTVSS (SEQ ID NO: 6006) |
| 15E1_ Humanize d variant_V H5 | QIQLVQS GAEVKK PGASVK VSCKVS GFSITTT (SEQ ID NO: 7334) | GYHWN (SEQ ID NO: 7313) | WVRQAP GQGLEW MG (SEQ ID NO: 6027) | YIYSSGS TSYNPSL KS (SEQ ID NO: 6001) | RVTMTR DTSTNTF YMELSS LRSEDT AVYYCA R (SEQ ID NO: 6037) | GDWHYF DY (SEQ ID NO: 7315) | WGQGT MVTVSS (SEQ ID NO: 6006) |

TABLE 8

Exemplary light chain CDRs and FWRs of NKp30-targeting antigen binding domains

| Ab ID | VLFWR1 | VLCDR1 | VLFWR2 | VLCDR2 | VLFWR3 | VLCDR3 | VLFWR4 |
|---|---|---|---|---|---|---|---|
| 9G1-LC | SYTLTQP PLLSVAL GHKATIT C (SEQ ID NO: 6066) | SGERLSD KYVH (SEQ ID NO: 6063) | WYQQKP GRAPVM VIY (SEQ ID NO: 6067) | ENDKRP S (SEQ ID NO: 6064) | GIPDQFS GSNSGNI ATLTISK AQAGYE ADYYC (SEQ ID NO: 7292) | QSWDST NSAV (SEQ ID NO: 7293) | FGSGTQ LTVL (SEQ ID NO: 6069) |
| 15H6-LC | SYTLTQP PSLSVAP GQKATII C (SEQ ID NO: 6073) | SGENLS DKYVH (SEQ ID NO: 6070) | WYQQKP GRAPVM VIY (SEQ ID NO: 6074) | ENEKRPS (SEQ ID NO: 6071) | GIPDQFS GSNSGNI ATLTISK AQPGSE ADYYC (SEQ ID NO: 6075) | HYWESI NSVV (SEQ ID NO: 6072) | FGSGTH LTVL (SEQ ID NO: 6076) |
| 9G1-LC_1 | QSVTTQ PPSVSGA PGQRVTI SC (SEQ ID NO: 6077) | SGERLSD KYVH (SEQ ID NO: 6063) | WYQQLP GTAPKM LIY (SEQ ID NO: 6078) | ENDKRP S (SEQ ID NO: 6064) | GVPDRF SGSNSG NSASLAI TGLQAE DEADYY C (SEQ ID NO: 6079) | QSWDST NSAV (SEQ ID NO: 7293) | FGGGTQ LTVL (SEQ ID NO: 6080) |
| 9G1-LC_2 | QSVTTQ PPSASGT PGQRVTI SC (SEQ ID NO: 6081) | SGERLSD KYVH (SEQ ID NO: 6063) | WYQQLP GTAPKM LIY (SEQ ID NO: 6082) | ENDKRP S (SEQ ID NO: 6064) | GVPDRF SGSNSG NSASLAI SGLQSE DEADYY C (SEQ ID NO: 6083) | QSWDST NSAV (SEQ ID NO: 7293) | FGGGTQ LTVL (SEQ ID NO: 6084) |
| 9G1-LC_3 | QSVTTQ PPSASGT PGQRVTI SC (SEQ ID NO: 6085) | SGERLSD KYVH (SEQ ID NO: 6063) | WYQQLP GTAPKM LIY (SEQ ID NO: 6086) | ENDKRP S (SEQ ID NO: 6064) | GVPDRF SGSNSG NSASLAI SGLRSED EADYYC (SEQ ID NO: 6087) | QSWDST NSAV (SEQ ID NO: 7293) | FGGGTQ LTVL (SEQ ID NO: 6088) |
| 9G1-LC_4 | SSETTQP HSVSVA TAQMAR ITC (SEQ ID NO: 6089) | SGERLSD KYVH (SEQ ID NO: 6063) | WYQQKP GQDPVM VIY (SEQ ID NO: 6090) | ENDKRP S (SEQ ID NO: 6064) | GIPERFS GSNPGN TATLTIS RIEAGDE ADYYC (SEQ ID NO: 6091) | QSWDST NSAV (SEQ ID NO: 7293) | FGGGTQ LTVL (SEQ ID NO: 6092) |
| 9G1-LC_5 | DIQMTQ SPSTLSA SVGDRV TITC (SEQ ID NO: 6093) | SGERLSD KYVH (SEQ ID NO: 6063) | WYQQKP GKAPKM LIY (SEQ ID NO: 6094) | ENDKRP S (SEQ ID NO: 6064) | GVPSRFS GSNSGN EATLTIS SLQPDDF ATYYC (SEQ ID NO: 6095) | QSWDST NSAV (SEQ ID NO: 7293) | FGQGTK VEIK (SEQ ID NO: 6096) |
| 15H6-LC_1 | QYVLTQ PPSASGT PGQRVTI SC (SEQ | SGENLS DKYVH (SEQ ID NO: 6070) | WYQQLP GTAPKM LIY (SEQ | ENEKRPS (SEQ ID NO: 6071) | GVPDRF SGSNSG NSASLAI SGLQSE | HYWESI NSVV (SEQ ID NO: 6072) | FGEGTEL TVL (SEQ ID NO: 6100) |
| | ID NO: 6097) | | ID NO: 6098) | | DEADYY C (SEQ ID NO: 6099) | | |
| 15H6-LC_2 | QYVLTQ PPSASGT PGQRVTI SC (SEQ ID NO: 6101) | SGENLS DKYVH (SEQ ID NO: 6070) | WYQQLP GTAPKM LIY (SEQ ID NO: 6102) | ENEKRPS (SEQ ID NO: 6071) | GVPDRF SGSNSG NSASLAI SGLRSED EADYYC (SEQ ID NO: 6103) | HYWESI NSVV (SEQ ID NO: 6072) | FGEGTEL TVL (SEQ ID NO: 6104) |
| 15H6-LC_3 | SYELTQP PSVSVSP GQTASIT C (SEQ ID NO: 6105) | SGENLS DKYVH (SEQ ID NO: 6070) | WYQQKP GQSPVM VIY (SEQ ID NO: 6106) | ENEKRPS (SEQ ID NO: 6071) | GIPERFS GSNSGN TATLTIS GTQAMD EADYYC (SEQ ID NO: 6107) | HYWESI NSVV (SEQ ID NO: 6072) | FGEGTEL TVL (SEQ ID NO: 6108) |
| 15H6-LC_4 | DYVLTQ SPLSLPV TPGEPAS ISC (SEQ ID NO: 6109) | SGENLS DKYVH (SEQ ID NO: 6070) | WYLQKP GQSPQM LIY (SEQ ID NO: 6110) | ENEKRPS (SEQ ID NO: 6071) | GVPDRF SGSNSG NDATLKI SRVEAE DVGVYY C (SEQ ID NO: 6111) | HYWESI NSVV (SEQ ID NO: 6072) | FGQGTK VEIK (SEQ ID NO: 6112) |
| 15H6-LC_5 | AYQLTQ SPSSLSA SVGDRV TITC (SEQ ID NO: 6113) | SGENLS DKYVH (SEQ ID NO: 6070) | WYQQKP GKAPKM LIY (SEQ ID NO: 6114) | ENEKRPS (SEQ ID NO: 6071) | GVPSRFS GSNSGN DATLTIS SLQPEDF ATYYC (SEQ ID NO: 6115) | HYWESI NSVV (SEQ ID NO: 6072) | FGQGTK VEIK (SEQ ID NO: 6116) |
| 15H6-LC_6 | EYVLTQ SPATLSV SPGERAT LSC (SEQ ID NO: 6117) | SGENLS DKYVH (SEQ ID NO: 6070) | WYQQKP GQAPRM LIY (SEQ ID NO: 6118) | ENEKRPS (SEQ ID NO: 6071) | GIPARFS GSNSGN EATLTIS SLQSEDF AVYYC (SEQ ID NO: 6119) | HYWESI NSVV (SEQ ID NO: 6072) | FGQGTK VEIK (SEQ ID NO: 6120) |
| 9D9-LC | SYTLTQP PLVSVA LGQKATI IC (SEQ ID NO: 7320) | SGENLS DKYVH (SEQ ID NO: 6070) | WYQQKP GRAPVM VIY (SEQ ID NO: 6067) | ENDKRP S (SEQ ID NO: 6064) | GIPDQFS GSNSGNI ATLTISK AQAGYE ADYYC (SEQ ID NO: 7292) | HCWDST NSAV (SEQ ID NO: 7321) | FGSGTH LTVL (SEQ ID NO: 6076) |
| 3A12-LC | SYTLTQP PLVSVA LGQKATI IC (SEQ ID NO: 7320) | SGENLS DKYVH (SEQ ID NO: 6070) | WYQQKP GRAPVM VIY (SEQ ID NO: 6067) | ENDKRP S (SEQ ID NO: 6064) | GIPDQFS GSNSGNI ATLTISK AQAGYE ADYYC (SEQ ID NO: 7292) | HCWDST NSAV (SEQ ID NO: 7321) | FGSGTH LTVL (SEQ ID NO: 6076) |
| 12D10-LC | SYTLTQP PSLSVAP GQKATII C (SEQ ID NO: 6073) | SGENLS DKYVH (SEQ ID NO: 6070) | WYQQKP GRAPVM VIY (SEQ ID NO: 6074) | ENEKRPS (SEQ ID NO: 6071) | GIPDQFS GSNSGNI ATLTISK AQPGSE ADYYC (SEQ ID NO: 6075) | HYWESI NSVV (SEQ ID NO: 6072) | FGSGTH LTVL (SEQ ID NO: 6076) |
| 15E1-LC | SFTLTQP PLVSVA VGQVAT ITC (SEQ ID NO: 7325) | SGEKLS DKYVH (SEQ ID NO: 7326) | WYQQKP GRAPVM VIY (SEQ ID NO: 6067) | ENDRRP S (SEQ ID NO: 7327) | GIPDQFS GSNSGNI ASLTISK AQAGDE ADYFC (SEQ ID NO: 7328) | QFWDST NSAV (SEQ ID NO: 7329) | FGGGTQ LTVL (SEQ ID NO: 6080) |
| 15E1_ Humanize d variant_V L1 | SSETTQP PSVSVSP GQTASIT C (SEQ ID NO: 7335) | SGEKLS DKYVH (SEQ ID NO: 7326) | WYQQKP GQSPVM VIY (SEQ ID NO: 6106) | ENDRRP S (SEQ ID NO: 7327) | GIPERFS GSNSGN TATLTIS GTQAMD EADYFC (SEQ ID NO: 7336) | QFWDST NSAV (SEQ ID NO: 7329) | FGGGTQ LTVL (SEQ ID NO: 6080) |
| 15E1_ Humanize d variant_V L2 | SSETTQP HSVSVA TAQMAR ITC (SEQ ID NO: 6089) | SGEKLS DKYVH (SEQ ID NO: 7326) | WYQQKP GQDPVM VIY (SEQ ID NO: 6090) | ENDRRP S (SEQ ID NO: 7327) | GIPERFS GSNPGN TATLTIS RIEAGDE ADYFC (SEQ ID NO: 7337) | QFWDST NSAV (SEQ ID NO: 7329) | FGGGTQ LTVL (SEQ ID NO: 6080) |
| 15E1_ Humanize d variant_V L3 | QSVTTQ PPSASGT PGQRVTI SC (SEQ ID NO: 6081) | SGEKLS DKYVH (SEQ ID NO: 7326) | WYQQLP GTAPKM LIY (SEQ ID NO: 6078) | ENDRRP S (SEQ ID NO: 7327) | GVPDRF SGSNSG NSASLAI SGLRSED EADYFC (SEQ ID NO: 7338) | QFWDST NSAV (SEQ ID NO: 7329) | FGGGTQ LTVL (SEQ ID NO: 6080) |
| 15E1_ Humanize d variant_V L4 | QSVTTQ PPSVSGA PGQRVTI SC (SEQ ID NO: 6077) | SGEKLS DKYVH (SEQ ID NO: 7326) | WYQQLP GTAPKM LIY (SEQ ID NO: 6078) | ENDRRP S (SEQ ID NO: 7327) | GVPDRF SGSNSG NSASLAI TGLQAE DEADYF C (SEQ ID NO: 7339) | QFWDST NSAV (SEQ ID NO: 7329) | FGGGTQ LTVL (SEQ ID NO: 6080) |
| 15E1_ Humanize d variant_V L5 | DSVTTQ SPLSLPV TLGQPA SISC (SEQ ID NO: 7340) | SGEKLS DKYVH (SEQ ID NO: 7326) | WYQQRP GQSPRM LIY (SEQ ID NO: 7341) | ENDRRP S (SEQ ID NO: 7327) | GVPDRF SGSNSG NDATLKI SRVEAE DVGVYF C (SEQ ID NO: 7342) | QFWDST NSAV (SEQ ID NO: 7329) | FGGGTK VEIK (SEQ ID NO: 233) |

TABLE 8A

Exemplary heavy chain CDRs and FWRs of NKp30-targeting antigen binding domains

| | | | | | | | |
|---|---|---|---|---|---|---|---|
| Ab ID | VHFWR1 | VHCDR1 | VHFWR2 | VHCDR2 | VHFWR3 | VHCDR3 | VHFWR4 |
| BKM0138 | EIQLLES GGGLVQ PGGSLRL SCAVSG FS (SEQ ID NO: 18) | ITTTGYH WN (SEQ ID NO: 19) | WVRGAP GKGLEW VG (SEQ ID NO: 20) | YIYSSGS TSYNPSL KS (SEQ ID NO: 21) | RFTISRD TSKNTF YLQMNS LRAEDT AVYYCA R (SEQ ID NO: 22) | GDWHYF DY (SEQ ID NO: 23) | WGQGT MVTVSS (SEQ ID NO: 24) |
| BKM0139 | EIQLLES GGGLVQ PGGSLRL SCAVSG FS (SEQ ID NO: 32) | ITTTGYH WN (SEQ ID NO: 33) | WVRQAP GKGLEW VG (SEQ ID NO: 34) | YIYSSGS TSYNPSL KS (SEQ ID NO: 35) | RFTISRD TSKNTF YLQMNS LRAEDT AVYYCA R (SEQ ID NO: 36) | GDWHYF DY (SEQ ID NO: 37) | WGQGT MVTVSS (SEQ ID NO: 38) |
| BKM0140 | EIQLLES GGGLVQ PGGSLRL SCAVSG FS (SEQ ID NO: 46) | ITTTGYH WN (SEQ ID NO: 47) | WVRQAP GKGLEW VG (SEQ ID NO: 48) | YIYSSGS TSYNPSL KS (SEQ ID NO: 49) | RFTISRD TSKNTF YLQMNS LRAEDT AVYYCA R (SEQ ID NO: 50) | GDWHYF DY (SEQ ID NO: 51) | WGQGT MVTVSS (SEQ ID NO: 52) |
| BKM0141 | EIQLLES GGGLVQ PGGSLRL SCAVSG FS (SEQ ID NO: 60) | ITTTGYH WN (SEQ ID NO: 61) | WVRQAP GKGLEW VG (SEQ ID NO: 62) | YIYSSGS TSYNPSL KS (SEQ ID NO: 63) | RFTISRD TSKNTF YLQMNS LRAEDT AVYYCA R (SEQ ID NO: 64) | GDWHYF DY (SEQ ID NO: 65) | WGQGT MVTVSS (SEQ ID NO: 66) |
| BKM0142 | EIQLLES GGGLVQ PGGSLRL SCAVSG FS (SEQ ID NO: 74) | ITTTGYH WN (SEQ ID NO: 7397) | WVRQAP GKGLEW VG (SEQ ID NO: 7398) | YIYSSGS TSYAPSL KS (SEQ ID NO: 7399) | RFTISRD TSKNTF YLQMNS LRAEDT AVYYCA R (SEQ ID NO: 7400) | GDWHYF DY (SEQ ID NO: 79) | WGQGT MVTVSS (SEQ ID NO: 80) |
| BKM0143 | EIQLLES GGGLVQ PGGSLRL SCAVSG FS (SEQ ID NO: 88) | ITTTGYH WN (SEQ ID NO: 89) | WVRQAP GKGLEW VG (SEQ ID NO: 90) | YIYSSGS TSYAPSL KS (SEQ ID NO: 91) | RFTISRD TSKNTF YLQMNS LRAEDT AVYYCA R (SEQ ID NO: 92) | GDWHYF DY (SEQ ID NO: 93) | WGQGT MVTVSS (SEQ ID NO: 94) |
| BKM0144 | EIQLLES GGGLVQ PGGSLRL SCAVSG FS (SEQ ID NO: 102) | ITTTGYH WN (SEQ ID NO: 103) | WVRQAP GKGLEW VG (SEQ ID NO: 104) | YIYSSGS TSYAPSL KS (SEQ ID NO: 105) | RFTISRD TSKNTF YLQMNS LRAEDT AVYYCA R (SEQ ID NO: 106) | GDWHYF DY (SEQ ID NO: 107) | WGQGT MVTVSS (SEQ ID NO: 108) |
| BKM0145 | EIQLLES GGGLVQ PGGSLRL SCAVSG FS (SEQ ID NO: 115) | ITTTGYH WN (SEQ ID NO: 116) | WVRQAP GKGLEW VG (SEQ ID NO: 117) | YIYSSGS TSYAPSL KS (SEQ ID NO: 118) | RFTISRD TSKNTF YLQMNS LRAEDT AVYYCA R (SEQ ID NO: 119) | GDWHYF DY (SEQ ID NO: 120) | WGQGT MVTVSS (SEQ ID NO: 121) |

TABLE 8B

Exemplary light chain CDRs and FWRs of NKp30-targeting antigen binding domains

| | | | | | | | |
|---|---|---|---|---|---|---|---|
| Ab ID | VLFWR1 | VLCDR1 | VLFWR2 | VLCDR2 | VLFWR3 | VLCDR3 | VLFWR4 |
| BKM0138 | DSVTTQ SPLSLPV TLGQPA SISC (SEQ ID NO: 25) | SGEKLS DKYVH (SEQ ID NO: 26) | WYQQRP GQSPRM LIY (SEQ ID NO: 27) | ENDRRP S (SEQ ID NO: 28) | GVPDRF SGSNSG NDATLKI SRVEAE DVGVYF C (SEQ ID NO: 29) | QFWDST ASAV (SEQ ID NO: 30) | FGGGTK VEIK (SEQ ID NO: 31) |
| BKM0139 | DSVTTQ SPLSLPV TLGQPA SISC (SEQ ID NO: 39) | SGEKLS DKYVH (SEQ ID NO: 40) | WYQQRP GQSPRM LIY (SEQ ID NO: 41) | ENDRRP S (SEQ ID NO: 7393) | GVPDRF, SGSNSG NDATLKI SRVEAE DVGVYF C (SEQ ID NO: 7394) | QFWAST NSAV (SEQ ID NO: 7395) | FGGGTK VEIK (SEQ ID NO: 7396) |
| BKM0140 | SSETTQP PSVSVSP GQTASIT C (SEQ ID NO: 53) | SGEKLS DKYVH (SEQ ID NO: 54) | WYQQKP GQSPVM VIY (SEQ ID NO: 55) | ENDRRP S (SEQ ID NO: 56) | GIPERFS GSNSGN TATLTIS GTQAMD EADYFC (SEQ ID NO: 57) | QFWAST N SAV (SEQ ID NO: 58) | FGGGTQ LTVL (SEQ ID NO: 59) |
| BKM0141 | SSETTQP PSVSVSP GQTASIT C (SEQ ID NO: 67) | SGEKLS DKYVH (SEQ ID NO: 68) | WYQQKP GQSPVM VIY (SEQ ID NO: 69) | ENDRRP S (SEQ ID NO: 70) | GIPERFS GSNSGN TATLTIS GTQAMD EADYFC (SEQ ID NO: 71) | QFWDST ASAV (SEQ ID NO: 72) | FGGGTQ LTVL (SEQ ID NO: 73) |
| BKM0142 | DSVTTQ SPLSLPV TLGQPA SISC (SEQ ID NO: 81) | SGEKLS DKYVH (SEQ ID NO: 82) | WYQQRP GQSPRM LIY (SEQ ID NO: 83) | ENDRRP S (SEQ ID NO: 84) | GVPDRF SGSNSG NDATLKI SRVEAE DVGVYF C (SEQ ID NO: 85) | QFWDST N SA V (SEQ ID NO: 86) | FGGGTK VEIK (SEQ ID NO: 87) |
| BKM0143 | SSETTQP PSVSVSP GQTASIT C (SEQ ID NO: 95) | SGEKLS DKYVH (SEQ ID NO: 96) | WYQQKP GQSPVM VIY (SEQ ID NO: 97) | ENDRRP S (SEQ ID NO: 98) | GIPERFS GSNSGN TATLTIS GTQAMD EADYFC (SEQ ID NO: 99) | QFWDST N SAV (SEQ ID NO: 100) | FGGGTQ LTVL (SEQ ID NO: 101) |
| BKM0144 | DSVTTQ SPLSLPV TLGQPA SISC (SEQ ID NO: 109) | SGEKLS DKYVH (SEQ ID NO: 110) | WY QQRP GQSPRM LIY (SEQ ID NO: 111) | ENDRRP S (SEQ ID NO: 112) | GVPDRF SGSNSG NDATLKI SRVEAE DVGVYF C (SEQ ID NO: 85) | QFWAST ASAV (SEQ ID NO: 113) | FGGGTK VEIK (SEQ ID NO: 114) |
| BKM0145 | SSETTQP PSVSVSP GQTASIT C (SEQ ID NO: 122) | SGEKLS DKYVH (SEQ ID NO: 123) | WYQQKP GQSPVM VIY (SEQ ID NO: 124) | ENDRRP S (SEQ ID NO: 125) | GIPERFS GSNSGN TATLTIS GTQAMD EADYFC (SEQ ID NO: 126) | QFWAST ASAV (SEQ ID NO: 127) | FGGGTQ LTVL (SEQ ID NO: 128) |

TABLE 9

Exemplary variable regions of NKp30-targeting antigen binding domains

| | | | |
|---|---|---|---|
| SEQ ID NO | Ab ID | Description | Sequence |
| SEQ ID NO: 6121 | 9G1-HC | 9G1 heavy chain variable region | QIQLQESGPGLVKPSQSLSLTCSVTGFSINTGGYHWNWI RQFPGKKLEWMGYIYSSGSTSYNPSLKSRISITRDTSKN QFFLQLNSVTTEDTATYYCARGNWHYFDFWGQGTMV TVSS |
| SEQ ID NO: 6122 | 15H6-HC | 15H6 heavy chain variable region | QIQLQESGPGLVKPSQSLSLTCSVTGFSINTGGYHWNWI RQFPGKKLEWMGYIYSSGTTRYNPSLKSRISITRDTSKN QFFLQLNSVTPEDTATYYCTRGNWHYFDYWGQGTLVA VSS |
| SEQ ID NO: 6123 | 9G1-HC_1 | 9G1 heavy chain variable region humanized variant 1 | QIQLQESGPGLVKPSETLSLTCTVSGFSINTGGYHWNWI RQPAGKGLEWIGYIYSSGSTSYNPSLKSRVTMSRDTSK NQFSLKLSSVTAADTAVYYCARGNWHYFDFWGQGTM VTVSS |
| SEQ ID NO: 6124 | 9G1-HC_2 | 9G1 heavy chain variable region humanized variant 2 | QIQLQESGPGLVKPSQTLSLTCTVSGFSINTGGYHWNWI RQHPGKGLEWIGYIYSSGSTSYNPSLKSLVTISRDTSKN QFSLKLSSVTAADTAVYYCARGNWHYFDFWGQGTMV TVSS |
| SEQ ID NO: 6125 | 9G1-HC_3 | 9G1 heavy chain variable region humanized variant 3 | EIQLLESGGGLVQPGGSLRLSCAVSGFSINTGGYHWNW VRQAPGKGLEWVGYIYSSGSTSYNPSLKSRFTISRDTSK NTFYLQMNSLRAEDTAVYYCARGNWHYFDFWGQGT MVTVSS |
| SEQ ID NO: 6126 | 9G1-HC_4 | 9G1 heavy chain variable region humanized variant 4 | QIQLVQSGAEVKKPGSSVKVSCKVSGFSINTGGYHWN WVRQAPGQGLEWMGYIYSSGSTSYNPSLKSRVTITRDT STNTFYMELSSLRSEDTAVYYCARGNWHYFDFWGQGT MVTVSS |
| SEQ ID NO: 6127 | 9G1-HC_5 | 9G1 heavy chain variable region humanized variant 5 | EIQLVESGGGLVQPGGSLRLSCAVSGFSINTGGYHWNW VRQAPGKGLEWVGYIYSSGSTSYNPSLKSRFTISRDTAK NSFYLQMNSLRAEDTAVYYCARGNWHYFDFWGQGTM VTVSS |
| SEQ ID NO: 6128 | 9G1-HC_6 | 9G1 heavy chain variable region humanized variant 6 | QIQLVQSGAEVKKPGASVKVSCKVSGFSINTGGYHWN WVRQAPGQGLEWMGYIYSSGSTSYNPSLKSRVTMTRD TSTNTFYMELSSLRSEDTAVYYCARGNWHYFDFWGQG TMVTVSS |
| SEQ ID NO: 6129 | 15H6-HC_1 | 15H6 heavy chain variable region humanized variant 1 | QIQLQESGPGLVKPSQTLSLTCTVSGFSINTGGYHWNWI RQHPGKGLEWIGYIYSSGTTRYNPSLKSLVTISRDTSKN QFSLKLSSVTAADTAVYYCARGNWHYFDYWGQGTLV TVSS |
| SEQ ID NO: 6130 | 15H6-HC_2 | 15H6 heavy chain variable region humanized variant 2 | QIQLQESGPGLVKPSETLSLTCTVSGFSINTGGYHWNWI RQPAGKGLEWIGYIYSSGTTRYNPSLKSRVTMSRDTSK NQFSLKLSSVTAADTAVYYCARGNWHYFDYWGQGTL VTVSS |
| SEQ ID NO: 6131 | 15H6-HC_3 | 15H6 heavy chain variable region humanized variant 3 | EIQLLESGGGLVQPGGSLRLSCAVSGFSINTGGYHWNW VRQAPGKGLEWVGYIYSSGTTRYNPSLKSRFTISRDTSK NTFYLQMNSLRAEDTAVYYCARGNWHYFDYWGQGTL VTVSS |
| SEQ ID NO: 6132 | 15H6-HC_4 | 15H6 heavy chain variable region humanized variant 4 | QIQLVESGGGLVKPGGSLRLSCAVSGFSINTGGYHWNW IRQAPGKGLEWVGYIYSSGTTRYNPSLKSRFTISRDTAK NSFYLQMNSLRAEDTAVYYCARGNWHYFDYWGQGTL VTVSS |
| SEQ ID NO: 6133 | 15H6-HC_5 | 15H6 heavy chain variable region humanized variant 5 | QIQLVQSGAEVKKPGASVKVSCKVSGFSINTGGYHWN WVRQAPGQGLEWMGYIYSSGTTRYNPSLKSRVTMTRD TSTNTFYMELSSLRSEDTAVYYCARGNWHYFDYWGQ GTLVTVSS |
| SEQ ID NO: 6134 | 15H6-HC_6 | 15H6 heavy chain variable region humanized variant 6 | EIQLVQSGAEVKKPGATVKISCKVSGFSINTGGYHWNW VQQAPGKGLEWMGYIYSSGTTRYNPSLKSRVTITRDTS TNTFYMELSSLRSEDTAVYYCARGNWHYFDYWGQGT LVTVSS |
| SEQ ID NO: 7294 | 9G1-LC | 9G1 light chain variable region | SYTLTQPPLLSVALGHKATITCSGERLSDKYVHWYQQK PGRAPVMVIYENDKRPSGIPDQFSGSNSGNIATLTISKA QAGYEADYYCQSWDSTNSAVFGSGTQLTVL |
| SEQ ID NO: 6136 | 15H6-LC | 15H6 light chain variable region | SYTLTQPPSLSVAPGQKATIICSGENLSDKYVHWYQQK PGRAPVMVIYENEKRPSGIPDQFSGSNSGNIATLTISKAQ PGSEADYYCHYWESINSVVFGSGTHLTVL |
| SEQ ID NO: 6137 | 9G1-LC_1 | 9G1 light chain variable region humanized variant 1 | QSVTTQPPSVSGAPGQRVTISCSGERLSDKYVHWYQQL PGTAPKMLIYENDKRPSGVPDRFSGSNSGNSASLAITGL QAEDEADYYCQSWDSTNSAVFGGGTQLTVL |
| SEQ ID NO: 6138 | 9G1-LC_2 | 9G1 light chain variable region humanized variant 2 | QSVTTQPPSASGTPGQRVTISCSGERLSDKYVHWYQQL PGTAPKMLIYENDKRPSGVPDRFSGSNSGNSASLAISGL QSEDEADYYCQSWDSTNSAVFGGGTQLTVL |
| SEQ ID NO: 6139 | 9G1-LC_3 | 9G1 light chain variable region | QSVTTQPPSASGTPGQRVTISCSGERLSDKYVHWYQQL PGTAPKMLIYENDKRPSGVPDRFSGSNSGNSASLAISGL RSEDEADYYCQSWDSTNSAVFGGGTQLTVL |
| | | humanized variant 3 | |
| SEQ ID NO: 6140 | 9G1-LC_4 | 9G1 light chain variable region humanized variant 4 | SSETTQPHSVSVATAQMARITCSGERLSDKYVHWYQQ KPGQDPVMVIYENDKRPSGIPERFSGSNPGNTATLTISRI EAGDEADYYCQSWDSTNSAVFGGGTQLTVL |
| SEQ ID NO: 6141 | 9G1-LC_5 | 9G1 light chain variable region humanized variant 5 | DIQMTQSPSTLSASVGDRVTITCSGERLSDKYVHWYQQ KPGKAPKMLIYENDKRPSGVPSRFSGSNSGNEATLTISS LQPDDFATYYCQSWDSTNSAVFGQGTKVEIK |
| SEQ ID NO: 6142 | 15H6-LC_1 | 15H6 light chain variable region humanized variant 1 | QYVLTQPPSASGTPGQRVTISCSGENLSDKYVHWYQQL PGTAPKMLIYENEKRPSGVPDRFSGSNSGNSASLAISGL QSEDEADYYCHYWESINSVVFGEGTELTVL |
| SEQ ID NO: 6143 | 15H6-LC_2 | 15H6 light chain variable region humanized variant 2 | QYVLTQPPSASGTPGQRVTISCSGENLSDKYVHWYQQL PGTAPKMLIYENEKRPSGVPDRFSGSNSGNSASLAISGL RSEDEADYYCHYWESINSVVFGEGTELTVL |
| SEQ ID NO: 6144 | 15H6-LC_3 | 15H6 light chain variable region humanized variant 3 | SYELTQPPSVSVSPGQTASITCSGENLSDKYVHWYQQK PGQSPVMVIYENEKRPSGIPERFSGSNSGNTATLTISGTQ AMDEADYYCHYWESINSVVFGEGTELTVL |
| SEQ ID NO: 6145 | 15H6-LC_4 | 15H6 light chain variable region humanized variant 4 | DYVLTQSPLSLPVTPGEPASISCSGENLSDKYVHWYLQ KPGQSPQMLIYENEKRPSGVPDRFSGSNSGNDATLKISR VEAEDVGVYYCHYWESINSVVFGQGTKVEIK |
| SEQ ID NO: 6146 | 15H6-LC_5 | 15H6 light chain variable region humanized variant 5 | AYQLTQSPSSLSASVGDRVTITCSGENLSDKYVHWYQQ KPGKAPKMLIYENEKRPSGVPSRFSGSNSGNDATLTISS LQPEDFATYYCHYWESINSVVFGQGTKVEIK |
| SEQ ID NO: 6147 | 15H6-LC_6 | 15H6 light chain variable region humanized variant 6 | EYVLTQSPATLSVSPGERATLSCSGENLSDKYVHWYQQ KPGQAPRMLIYENEKRPSGIPARFSGSNSGNEATLTISSL QSEDFAVYYCHYWESINSVVFGQGTKVEIK |
| SEQ ID NO: 7295 | 9D9-HC | 9D9 heavy chain variable region | QIQLQESGPGLVKPSQSLSLSCSVTGFSINTGGYHWNWI RQFPGKKVEWMGYIYSSGTTKYNPSLKSRISITRDTSKN QFFLQLNSVTTEDTATYYCARGDWHYFDYWGQGTMV AVSS |
| SEQ ID NO: 7296 | 9D9-LC | 9D9 light chain variable region | SYTLTQPPLVSVALGQKATIICSGENLSDKYVHWYQQK PGRAPVMVIYENDKRPSGIPDQFSGSNSGNIATLTISKA QAGYEADYYCHCWDSTNSAVFGSGTHLTVL |
| SEQ ID NO: 7297 | 3A12-HC | 3A12 heavy chain variable region | QIQLQESGPGLVKPSQSLSLTCSVTGFSINTGGYHWNWI RQFPGKKLEWMGYIYSSGSTRYNPSLKSRFSITRDTSKN QFFLQLNSVTTEDTATYYCTRGNWHYFDYWGQGTLV AVSS |
| SEQ ID NO: 7296 | 3A12-LC | 3A12 light chain variable region | SYTLTQPPLVSVALGQKATIICSGENLSDKYVHWYQQK PGRAPVMVIYENDKRPSGIPDQFSGSNSGNIATLTISKA QAGYEADYYCHCWDSTNSAVFGSGTHLTVL |
| SEQ ID NO: 6122 | 12D10-HC | 12D10 heavy chain variable region | QIQLQESGPGLVKPSQSLSLTCSVTGFSINTGGYHWNWI RQFPGKKLEWMGYIYSSGTTRYNPSLKSRISITRDTSKN QFFLQLNSVTPEDTATYYCTRGNWHYFDYWGQGTLVA VSS |
| SEQ ID NO: 6136 | 12D10-LC | 12D10 light chain variable region | SYTLTQPPSLSVAPGQKATIICSGENLSDKYVHWYQQK PGRAPVMVIYENEKRPSGIPDQFSGSNSGNIATLTISKAQ PGSEADYYCHYWESINSVVFGSGTHLTVL |
| SEQ ID NO: 7298 | 15E1-HC | 15E1 heavy chain variable region | QIQLQESGPGLVKPSQSLSLSCSVTGFSITTTGYHWNWI RQFPGKKLEWMGYIYSSGSTSYNPSLKSRFSITRDTSKN QFFLQLNSVTTEDTATYYCARGDWHYFDYWGPGTMV TVSS |
| SEQ ID NO: 7299 | 15E1-LC | 15E1 light chain variable region | SFTLTQPPLVSVAVGQVATITCSGEKLSDKYVHWYQQK PGRAPVMVIYENDRRPSGIPDQFSGSNSGNIASLTISKAQ AGDEADYFCQFWDSTNSAVFGGGTQLTVL |
| SEQ ID NO: 7300 | 15E1_ Humanized variant_VH 1 | 15E1 heavy chain variable region humanized variant 1 | QIQLQESGPGLVKPSQTLSLTCTVSGFSITTTGYHWNWI RQHPGKGLEWIGYIYSSGSTSYNPSLKSLVTISRDTSKN QFSLKLSSVTAADTAVYYCARGDWHYFDYWGQGTMV TVSS |
| SEQ ID NO: 7301 | 15E1_ Humanized variant_VH 2 | 15E1 heavy chain variable region humanized variant 2 | QIQLVESGGGLVKPGGSLRLSCAVSGFSITTTGYHWNW IRQAPGKGLEWVGYIYSSGSTSYNPSLKSRFTISRDTAK NSFYLQMNSLRAEDTAVYYCARGDWHYFDYWGQGT MVTVSS |
| SEQ ID NO: 7302 | 15E1_ Humanized variant_VH 3 (BJM0407 VH and BJM0411 VH) | 15E1 heavy chain variable region humanized variant 3 | EIQLLESGGGLVQPGGSLRLSCAVSGFSITTTGYHWNW VRQAPGKGLEWVGYIYSSGSTSYNPSLKSRFTISRDTSK NTFYLQMNSLRAEDTAVYYCARGDWHYFDYWGQGT MVTVSS |
| SEQ ID NO: 7303 | 15E1_ Humanized variant_VH 4 | 15E1 heavy chain variable region humanized variant 4 | EIQLVESGGGLVQPGGSLRLSCAVSGFSITTTGYHWNW VRQAPGKGLEWVGYIYSSGSTSYNPSLKSRFTISRDTAK NSFYLQMNSLRAEDTAVYYCARGDWHYFDYWGQGT MVTVSS |
| SEQ ID NO: 7304 | 15E1_ Humanized variant_VH 5 | 15E1 heavy chain variable region humanized variant 5 | QIQLVQSGAEVKKPGASVKVSCKVSGFSITTTGYHWN WVRQAPGQGLEWMGYIYSSGSTSYNPSLKSRVTMTRD TSTNTFYMELSSLRSEDTAVYYCARGDWHYFDYWGQ GTMVTVSS |
| SEQ ID NO: 7305 | 15E1_ Humanized variant_VL 1 (BJM0407 VL) | 15E1 light chain variable region humanized variant 1 | SSETTQPPSVSVSPGQTASITCSGEKLSDKYVHWYQQKP GQSPVMVIYENDRRPSGIPERFSGSNSGNTATLTISGTQ AMDEADYFCQFWDSTNSAVFGGGTQLTVL |
| SEQ ID NO: 7306 | 15E1_ Humanized variant_VL 2 | 15E1 light chain variable region humanized variant 2 | SSETTQPHSVSVATAQMARITCSGEKLSDKYVHWYQQ KPGQDPVMVIYENDRRPSGIPERFSGSNPGNTATLTISRI EAGDEADYFCQFWDSTNSAVFGGGTQLTVL |
| SEQ ID NO: 7307 | 15E1_ Humanized variant_VL 3 | 15E1 light chain variable region humanized variant 3 | QSVTTQPPSASGTPGQRVTISCSGEKLSDKYVHWYQQL PGTAPKMLIYENDRRPSGVPDRFSGSNSGNSASLAISGL RSEDEADYFCQFWDSTNSAVFGGGTQLTVL |
| SEQ ID NO: 7308 | 15E1_ Humanized variant_VL 4 | 15E1 light chain variable region humanized variant 4 | QSVTTQPPSVSGAPGQRVTISCSGEKLSDKYVHWYQQL PGTAPKMLIYENDRRPSGVPDRFSGSNSGNSASLAITGL QAEDEADYFCQFWDSTNSAVFGGGTQLTVL |
| SEQ ID NO: 7309 | 15E1_ Humanized variant_VL 5 (BJM0411 VL) | 15E1 light chain variable region humanized variant 5 | DSVTTQSPLSLPVTLGQPASISCSGEKLSDKYVHWYQQ RPGQSPRMLIYENDRRPSGVPDRFSGSNSGNDATLKISR VEAEDVGVYFCQFWDSTNSAVFGGGTKVEIK |
| SEQ ID NO: 1 | BKM0138 VH | BKM0138 heavy chain variable region | EIQLLESGGGLVQPGGSLRLSCAVSGFSITTTGYHWNW VRQAPGKGLEWVGYIYSSGSTSYNPSLKSRFTISRDTSK NTFYLQMNSLRAEDTAVYYCARGDWHYFDYWGQGT MVTVSS |
| SEQ ID NO: 2 | BKM0139 VH | BKM0139 heavy chain variable region | EIQLLESGGGLVQPGGSLRLSCAVSGFSITTTGYHWNW VRQAPGKGLEWVGYIY SSGSTSYNPSLKSRFTISRDTSK NTFYLQMNSLRAEDTAVYYCARGDWHYFDYWGQGT MVTVSS |
| SEQ ID NO: 3 | BKM0140 VH | BKM0140 heavy chain variable region | EIQLLESGGGLVQPGGSLRLSCAVSGFSITTTGYHWNW VRQAPGKGLEWVGYIYSSGSTSYNPSLKSRFTISRDTSK NTFYLQMNSLRAEDTAVYYCARGDWHYFDYWGQGT MVTVSS |
| SEQ ID NO: 4 | BKM0141 VH | BKM0141 heavy chain variable region | EIQLLESGGGLVQPGGSLRLSCAVSGFSITTTGYHWNW VRQAPGKGLEWVGYIYSSGSTSYNPSLKSRFTISRDTSK NTFYLQMNSLRAEDTAVYYCARGDWHYFDYWGQGT MVTVSS |
| SEQ ID NO: 5 | BKM0142 VH | BKM0142 heavy chain variable region | EIQLLESGGGLVQPGGSLRLSCAVSGFSITTTGYHWNW VRQAPGKGLEWVGYIY SSGSTSYAPSLKSRFTISRIDTSK NTFYLQMNSLRAEDTAVYYCARGDWHYFDYWGQGT MVTVSS |
| SEQ ID NO: 6 | BKM0143 VH | BKM0143 heavy chain variable region | EIQLLESGGGLVQPGGSLRLSCAVSGFSITTTGYHWNW VRQAPGKGLEWVGYIYSSGSTSYAPSLKSRFTISRDTSK NTFYLQMNSLRAEDTAVYYCARGDWHYFDYWGQGT MVTVSS |
| SEQ ID NO: 7 | BKM0144 VH | BKM0144 heavy chain variable region | EIQLLESGGGLVQPGGSLRLSCAVSGFSITTTGYHWNW VRQAPGKGLEWVGYIY SSGSTSYAPSLKSRFTISRDTSK NTFYLQMNSLRAEDTAVYYCARGDWHYFDYWGQGT MVTVSS |
| SEQ ID NO: 8 | BKM0145 VH | BKM0145 heavy chain variable region | EIQLLESGGGLVQPGGSLRLSCAVSGFSITTTGYHWNW VRQAPGKOLEWVGYIYSSGSTSYAPSLKSRFTISRDTSK |
| | | | NTFYLQMNSLRAEDTAVYYCARGDWHYFDYWGQGT MVTVSS |
| SEQ ID NO: 9 | BKM0138 VL | BKM0138 light chain variable region | DSVTTQSPLSLPVTLGQPASISCSGEKLSDKYVHWYQQ RPGQSPRMLIYENDRRPSGVPDRFSGSNSGNDATLKISR VEAEDVGVYFCQFWDSTASAVFGGGTKVEIK |
| SEQ ID NO: 10 | BKM0139 VL | BKM0139 light chain variable region | DSVTTQSPLSLPVTLGQPASISCSGEKLSDKYVHWYQQ RPGQSPRMLIYENDRRPSGVPDRFSGSNSGNDATLKISR VEAEDVGVYFCQFWASTNSAVFGGGTKVEIK |
| SEQ ID NO: 11 | BKM0140 VL | BKM0140 light chain variable region | SSETTQPPSVSVSPGQTASITCSGEKLSDKYVHWYQQKP GQSPVMVIYENDRRPSGIPERFSGSNSGNTATLTISGTQ AMDEADYFCQFWASTNSAVFGGGTQLTVL |
| SEQ ID NO: 12 | BKM0141 VL | BKM0141 light chain variable region | SSETTQPPSVSVSPGQTASITCSGEKLSDKYVHWYQQKP GQSPVMVIYENDRRPSGIPERFSGSNSGNTATLTISGTQ AMDEADYFCQFWDSTASAVFGGGTQLTVL |
| SEQ ID NO: 13 | BKM0142 VL | BKM0142 light chain variable region | DSVTTQSPLSLPVTLGQPASISCSGEKLSDKYVHWYQQ RPGQSPRMLIYENDRRPSGVPDRFSGSNSGNDATLKISR VEAEDVGVYFCQFWDSTNSAVFGGGTKVEIK |
| SEQ ID NO: 14 | BKM0143 VL | BKM0143 light chain variable region | SSETTQPPSVSVSPGQTASITCSGEKLSDKYVHWYQQKP GQSPVMVIYENDRRPSGIPERFSGSNSGNTATLTISGTQ AMDEADYFCQFWDSTNSAVFGGGTQLTVL |
| SEQ ID NO: 15 | BKM0144 VL | BKM0144 light chain variable region | DSVTTQSPLSLPVTLGQPASISCSGEKLSDKYVHWYQQ RPGQSPRMLIYENDRRPSGVPDRFSGSNSGNDATLKISR VEAEDVGVYFCQFWASTASAVFGGGTKVEIK |
| SEQ ID NO: 16 | BKM0145 VL | BKM0145 light chain variable region | SSETTQPPSVSVSPGQTASITCSGEKLSDKYVHWYQQKP GQSPVMVIYENDRRPSGIPERFSGSNSGNTATLTISGTQ AMDEADYFCQFWASTASAVFGGGTQLTVL |

TABLE 10

Exemplary NKp30-targeting antigen binding domains/antibody molecules

| | | | |
|---|---|---|---|
| SEQ ID NO | Ab ID | Description | Sequence |
| SEQ ID NO: 6148 | Ch(anti-NKp30 9G1)HC N297A | 9G1 heavy chain | QIQLQESGPGLVKPSQSLSLTCSVTGFSINTGGYHWNWIRQ FPGKKLEWMGYIYSSGSTSYNPSLKSRISITRDTSKNQFFL QLNSVTTEDTATYYCARGNWHYFDFWGQGTMVTVSSAS TKGPSVFPLAPSSKSTSGGTAALGCLVKDYFPEPVTVSWN SGALTSGVHTFPAVLQSSGLYSLSSVVTVPSSSLGTQTYIC NVNHKPSNTKVDKRVEPKSCDKTHTCPPCPAPELLGGPSV FLFPPKPKDTLMISRTPEVTCVVVDVSHEDPEVKFNWYVD GVEVHNAKTKPREEQYASTYRVVSVLTVLHQDWLNGKE YKCKVSNKALPAPIEKTISKAKGQPREPQVCTLPPSREEMT KNQVSLSCAVKGFYPSDIAVEWESNGQPENNYKTTPPVLD SDGSFFLVSKLTVDKSRWQQGNVFSCSVMHEALHNHYTQ KSLSLSPGK |
| SEQ ID NO: 6149 | Ch(anti-NKp30 9G1)HC | 9G1 heavy chain | QIQLQESGPGLVKPSQSLSLTCSVTGFSINTGGYHWNWIRQ FPGKKLEWMGYIYSSGSTSYNPSLKSRISITRDTSKNQFFL QLNSVTTEDTATYYCARGNWHYFDFWGQGTMVTVSSAS TKGPSVFPLAPSSKSTSGGTAALGCLVKDYFPEPVTVSWN SGALTSGVHTFPAVLQSSGLYSLSSVVTVPSSSLGTQTYIC NVNHKPSNTKVDKRVEPKSCDKTHTCPPCPAPELLGGPSV FLFPPKPKDTLMISRTPEVTCVVVDVSHEDPEVKFNWYVD GVEVHNAKTKPREEQYNSTYRVVSVLTVLHQDWLNGKE YKCKVSNKALPAPIEKTISKAKGQPREPQVCTLPPSREEMT |
| | | | KNQVSLSCAVKGFYPSDIAVEWESNGQPENNYKTTPPVLD SDGSFFLVSKLTVDKSRWQQGNVFSCSVMHEALHNHYTQ KSLSLSPGK |
| SEQ ID NO: 6150 | Ch(anti-NKp30 9G1)LC | 9G1 light chain | SYTLTQPPLLSVALGHKATITCSGERLSDKYVHWYQQKPG RAPVMVIYENDKRPSGIPDQFSGSNSGNIATLTISKAQAGY EADYYCQSWDSTNSAVFGSGTQLTVLGQPKANPTVTLFPP SSEELQANKATLVCLISDFYPGAVTVAWKADGSPVKAGV ETTKPSKQSNNKYAASSYLSLTPEQWKSHRSYSCQVTHEG STVEKTVAPTECS |
| SEQ ID NO: 6151 | Ch(anti-NKp30 15H6)HC N297A | 15H6 heavy chain | QIQLQESGPGLVKPSQSLSLTCSVTGFSINTGGYHWNWIRQ FPGKKLEWMGYIYSSGTTRYNPSLKSRISITRDTSKNQFFL QLNSVTPEDTATYYCTRGNWHYFDYWGQGTLVAVSSAS TKGPSVFPLAPSSKSTSGGTAALGCLVKDYFPEPVTVSWN SGALTSGVHTFPAVLQSSGLYSLSSVVTVPSSSLGTQTYIC NVNHKPSNTKVDKRVEPKSCDKTHTCPPCPAPELLGGPSV FLFPPKPKDTLMISRTPEVTCVVVDVSHEDPEVKFNWYVD GVEVHNAKTKPREEQYASTYRVVSVLTVLHQDWLNGKE YKCKVSNKALPAPIEKTISKAKGQPREPQVCTLPPSREEMT KNQVSLSCAVKGFYPSDIAVEWESNGQPENNYKTTPPVLD SDGSFFLVSKLTVDKSRWQQGNVFSCSVMHEALHNHYTQ KSLSLSPGK |
| SEQ ID NO: 6152 | Ch(anti-NKp30 15H6)HC (hole) | 15H6 heavy chain | QIQLQESGPGLVKPSQSLSLTCSVTGFSINTGGYHWNWIRQ FPGKKLEWMGYIYSSGTTRYNPSLKSRISITRDTSKNQFFL QLNSVTPEDTATYYCTRGNWHYFDYWGQGTLVAVSSAS TKGPSVFPLAPSSKSTSGGTAALGCLVKDYFPEPVTVSWN SGALTSGVHTFPAVLQSSGLYSLSSVVTVPSSSLGTQTYIC NVNHKPSNTKVDKRVEPKSCDKTHTCPPCPAPELLGGPSV FLFPPKPKDTLMISRTPEVTCVVVDVSHEDPEVKFNWYVD GVEVHNAKTKPREEQYNSTYRVVSVLTVLHQDWLNGKE YKCKVSNKALPAPIEKTISKAKGQPREPQVCTLPPSREEMT KNQVSLSCAVKGFYPSDIAVEWESNGQPENNYKTTPPVLD SDGSFFLVSKLTVDKSRWQQGNVFSCSVMHEALHNHYTQ KSLSLSPGK |
| SEQ ID NO: 6153 | Ch(anti-NKp30 15H6)LC | 15H6 light chain | SYTLTQPPSLSVAPGQKATIICSGENLSDKYVHWYQQKPG RAPVMVIYENEKRPSGIPDQFSGSNSGNIATLTISKAQPGSE ADYYCHYWESINSVVFGSGTHLTVLGQPKANPTVTLFPPS SEELQANKATLVCLISDFYPGAVTVAWKADGSPVKAGVE TTKPSKQSNNKYAASSYLSLTPEQWKSHRSYSCQVTHEGS TVEKTVAPTECS |
| SEQ ID NO: 7310 | BJM0859 lambda scFv | | EIQLLESGGGLVQPGGSLRLSCAVSGFSITTTGYHWNWVR QAPGKGLEWVGYIYSSGSTSYNPSLKSRFTISRDTSKNTFY LQMNSLRAEDTAVYYCARGDWHYFDYWGQGTMVTVSS GGGGSGGGGSGGGGSGGGGSSSETTQPPSVSVSPGQTASI TCSGEKLSDKYVHWYQQKPGQSPVMVIYENDRRPSGIPER FSGSNSGNTATLTISGTQAMDEADYFCQFWDSTNSAVFGG GTQLTVL |
| SEQ ID NO: 7311 | BJM0860 kappa scFv | | EIQLLESGGGLVQPGGSLRLSCAVSGFSITTTGYHWNWVR QAPGKGLEWVGYIYSSGSTSYNPSLKSRFTISRDTSKNTFY LQMNSLRAEDTAVYYCARGDWHYFDYWGQGTMVTVSS GGGGSGGGGSGGGGSGGGGSDSVTTQSPLSLPVTLGQPAS ISCSGEKLSDKYVHWYQQRPGQSPRMLIYENDRRPSGVPD RFSGSNSGNDATLKISRVEAEDVGVYFCQFWDSTNSAVFG GGTKVEIK |
| SEQ ID NO: 17 | BKM0138 scFv | | EIQLLESGGGLVQPGGSLRLSCAVSGFSITTTGYHWNWVR QAPGKGLEWVGYIYSSGSTSYNPSLKSRFTISRDTSKNTFY LQMNSLRAEDTAVYYCARGDWHYFDYWGQGTMVTVSS GGGGSGGGGSGGGGSGGGGSDSVTTQSPLSLPVTLGQPAS ISCSGEKLSDKYVHWYQQRPGQSPRMLIYENDRRPSGVPD RFSGSNSGNDATLKISRVEAEDVGVYFCQFWDSTASAVFG GGTKVEIK |
| SEQ ID NO: 7386 | BKM0139 scFv | | EIQLLESGGGLVQPGGSLRLSCAVSGFSITTTGYHWNWVR QAPGKGLEWVGYIYSSGSTSYNPSLKSRFTISRDTSKNTFY LQMNSLRAEDTAVYYCARGDWHYFDYWGQGTMVTVSS GGGGSGGGGSGGGGSGGGGSDSVTTQSPLSLPVTLGQPAS ISCSGEKLSDKYVHWYQQRPGQSPRMLIYENDRRPSGVPD RFSGSNSGNDATLKISRVEAEDVGVYFCQFWASTNSAVFG GGTKVEIK |
| SEQ ID NO: 7387 | BKM0140 scFv | | EIQLLESGGGLVQPGGSLRLSCAVSGFSITTTGYHWNWVR QAPGKGLEWVGYIYSSGSTSYNPSLKSRFTISRDTSKNTFY LQMNSLRAEDTAVYYCARGDWHYFDYWGQGTMVTVSS GGGGSGGGGSGGGGSGGGGSSSETTQPPSVSVSPGQTASI TCSGEKLSDKYVHWYQQKPGQSPVMVIYENDRRPSGIPER FSGSNSGNTATLTISGTQAMDEADYFCQFWASTNSAVFGG GTQLTVL |
| SEQ ID NO: 7388 | BKM0141 scFv | | EIQLLESGGGLVQPGGSLRLSCAVSGFSITTTGYHWNWVR QAPGKGLEWVGYIYSSGSTSYNPSLKSRFTISRDTSKNTFY LQMNSLRAEDTAVYYCARGDWHYFDYWGQGTMVTVSS GGGGSGGGGSGGGGSGGGGSSSETTQPPSVSVSPGQTASI TCSGEKLSDKYVHWYQQKPGQSPVMVIYENDRRPSGIPER FSGSNSGNTATLTISGTQAMDEADYFCQFWDSTASAVFGG GTQLTVL |
| SEQ ID NO: 7389 | BKM0142 scFv | | EIQLLESGGGLVQPGGSLRLSCAVSGFSITTTGYHWNWVR QAPGKGLEWVGYIYSSGSTSYAPSLKSRFTISRDTSKNTFY LQMNSLRAEDTAVYYCARGDWHYFDYWGQGTMVTVSS GGGGSGGGGSGGGGSGGGGSDSVTTQSPLSLPVTLGQPAS ISCSGEKLSDKYVHWYQQRPGQSPRMLIYENDRRPSGVPD RFSGSNSGNDATLKISRVEAEDVGVYFCQFWDSTNSAVFG GGTKVEIK |
| SEQ ID NO: 7390 | BKM0143 scFv | | EIQLLESGGGLVQPGGSLRLSCAVSGFSITTTGYHWNWVR QAPGKGLEWVGYIYSSGSTSYAPSLKSRFTISRDTSKNTFY LQMNSLRAEDTAVYYCARGDWHYFDYWGQGTMVTVSS GGGGSGGGGSGGGGSGGGGSSSETTQPPSVSVSPGQTASI TCSGEKLSDKYVHWYQQKPGQSPVMVIYENDRRPSGIPER FSGSNSGNTATLTISGTQAMDEADYFCQFWDSTNSAVFGG GTQLTVL |
| SEQ ID NO: 7391 | BKM0144 scFv | | EIQLLESGGGLVQPGGSLRLSCAVSGFSITTTGYHWNWVR QAPGKGLEWVGYIYSSGSTSYAPSLKSRFTISRDTSKNTFY LQMNSLRAEDTAVYYCARGDWHYFDYWGQGTMVTVSS GGGGSGGGGSGGGGSGGGGSDSVTTQSPLSLPVTLGQPAS ISCSGEKLSDKYVHWYQQRPGQSPRMLIYENDRRPSGVPD RFSGSNSGNDATLKISRVEAEDVGVYFCQFWASTASAVFG GGTKVEIK |
| SEQ ID NO: 7392 | BKM0145 scFv | | EIQLLESGGGLVQPGGSLRLSCAVSGFSITTTGYHWNWVR QAPGKGLEWVGYIYSSGSTSYAPSLKSRFTISRDTSKNTFY LQMNSLRAEDTAVYYCARGDWHYFDYWGQGTMVTVSS GGGGSGGGGSGGGGSGGGGSSSETTQPPSVSVSPGQTASI |
| | | | TCSGEKLSDKYVHWYQQKPGQSPVMVIYENDRRPSGIPER FSGSNSGNTATLTISGTQAMDEADYFCQFWASTASAVFGG GTQLTVL |

In some embodiments, the NK cell engager is an antigen binding domain that binds to NKp46 (e.g., NKp46 present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to NKp46, to the NK cell activates the NK cell. An antigen binding domain that binds to NKp46 (e.g., NKp46 present, e.g., expressed or displayed, on the surface of an NK cell) may be said to target NKp46, the NK cell, or both.

In some embodiments, the NK cell engager is an antigen binding domain that binds to NKG2D (e.g., NKG2D present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to NKG2D, to the NK cell activates the NK cell. An antigen binding domain that binds to NKG2D (e.g., NKG2D present, e.g., expressed or displayed, on the surface of an NK cell) may be said to target NKG2D, the NK cell, or both.

In some embodiments, the NK cell engager is an antigen binding domain that binds to CD16 (e.g., CD16 present, e.g., expressed or displayed, on the surface of an NK cell) and comprises any CDR amino acid sequence, framework region (FWR) amino acid sequence, or variable region amino acid sequence disclosed in Table 15. In some embodiments, binding of the NK cell engager, e.g., antigen binding domain that binds to CD16, to the NK cell activates the NK cell. An antigen binding domain that binds to CD16 (e.g., CD16 present, e.g., expressed or displayed, on the surface of an NK cell) may be said to target CD16, the NK cell, or both.

TABLE 15

Exemplary variable regions of NKp46, NKG2D, or CD16-targeting antigen binding domains

| | | | |
|---|---|---|---|
| SEQ ID NO | Ab ID | Description | Sequence |
| SEQ ID NO: 6175 | NKG2D_ 1scFV | scFv that binds NKG2D | QVHLQESGPGLVKPSETLSLTCTVSDDSISSYYWSWIR QPPGKGLEWIGHISYSGSANYNPSLKSRVTISVDTSKN QFSLKLSSVTAADTAVYYCANWDDAFNIWGQGTMVT VSSGGGGSGGGGSGGGGSGGGGSEIVLTQSPGTLSLSP GERATLSCRASQSVSSSYLAWYQQKPGQAPRLLIYGA SSRATGIPDRFSGSGSGTDFTLTISRLEPEDFAVYYCQQ YGSSPWTFGQGTKVEIK |
| SEQ ID NO: 6176 | NKG2D_ 1VH | VH that binds NKG2D | QVHLQESGPGLVKPSETLSLTCTVSDDSISSYYWSWIR QPPGKGLEWIGHISYSGSANYNPSLKSRVTISVDTSKN QFSLKLSSVTAADTAVYYCANWDDAFNIWGQGTMVT VSS |
| SEQ ID NO: 6177 | NKG2D_ 1VL | VL that binds NKG2D | EIVLTQSPGTLSLSPGERATLSCRASQSVSSSYLAWYQ QKPGQAPRLLIYGASSRATGIPDRFSGSGSGTDFTLTIS RLEPEDFAVYYCQQYGSSPWTFGQGTKVEIK |
| SEQ ID NO: 6178 | NKG2D_ 2scFv | scFv that binds NKG2D | EVQLVQSGAEVKEPGESLKISCKNSGYSFTNYWVGWV RQMPGKGLEWMGIIYPGDSDTRYSPSFQGQVTISADKS INTAYLQWSSLKASDTAMYYCGRLTMFRGIIIGYFDY WGQGTLVTVSSGGGGSGGGGSGGGGSGGGGSEIVLT QSPATLSLSPGERATLSCRASQSVSSYLAWYQQKPGQ APRLLIYDASNRATGIPARFSGSGSGTDFTLTISSLEPED FAVYYCQQRSNWPWTFGQGTKVEIK |
| SEQ ID NO: 6179 | NKG2D_ 2VH | VH that binds NKG2D | EVQLVQSGAEVKEPGESLKISCKNSGYSFTNYWVGWV RQMPGKGLEWMGIIYPGDSDTRYSPSFQGQVTISADKS INTAYLQWSSLKASDTAMYYCGRLTMFRGIIIGYFDY WGQGTLVTVSS |
| SEQ ID NO: 6180 | NKG2D_ 2VL | VL that binds NKG2D | EIVLTQSPATLSLSPGERATLSCRASQSVSSYLAWYQQ KPGQAPRLLIYDASNRATGIPARFSGSGSGTDFTLTISSL EPEDFAVYYCQQRSNWPWTFGQGTKVEIK |
| SEQ ID NO: 6181 | NKp46scF v | scFv that binds NKp46 | QVQLQQSGPELVKPGASVKMSCKASGYTFTDYVINW GKQRSGQGLEWIGEIYPGSGTNYYNEKFKAKATLTAD KSSNIAYMQLSSLTSEDSAVYFCARRGRYGLYAMDY WGQGTSVTVSSGGGGSGGGGSGGGGSGGGGSDIQMT QTTSSLSASLGDRVTISCRASQDISNYLNWYQQKPDGT VKLLIYYTSRLHSGVPSRFSGSGSGTDYSLTINNLEQED IATYFCQQGNTRPWTFGGGTKLEIK |
| SEQ ID NO: 6182 | NKp46V H | VH that binds NKp46 | QVQLQQSGPELVKPGASVKMSCKASGYTFTDYVINW GKQRSGQGLEWIGEIYPGSGTNYYNEKFKAKATLTAD KSSNIAYMQLSSLTSEDSAVYFCARRGRYGLYAMDY WGQGTSVTVSS |
| SEQ ID NO: 6183 | NKp46VL | VL that binds NKp46 | DIQMTQTTSSLSASLGDRVTISCRASQDISNYLNWYQQ KPDGTVKLLIYYTSRLHSGVPSRFSGSGSGTDYSLTINN LEQEDIATYFCQQGNTRPWTFGGGTKLEIK |
| SEQ ID NO: 6184 | CD16scFv | scFv that binds CD16 | EVQLVESGGGVVRPGGSLRLSCAASGFTFDDYGMSW VRQAPGKGLEWVSGINWNGGSTGYADSVKGRFTISRD NAKNSLYLQMNSLRAEDTAVYYCARGRSLLFDYWGQ GTLVTVSRGGGGSGGGGSGGGGSSELTQDPAVSVALG QTVRITCQGDSLRSYYASWYQQKPGQAPVLVIYGKNN RPSGIPDRFSGSSSGNTASLTITGAQAEDEADYYCNSRD SSGNHVVFGGGTKLTVL |
| SEQ ID NO: 6185 | CD16VH | VH that binds CD16 | EVQLVESGGGVVRPGGSLRLSCAASGFTFDDYGMSW VRQAPGKGLEWVSGINWNGGSTGYADSVKGRFTISRD NAKNSLYLQMNSLRAEDTAVYYCARGRSLLFDYWGQ GTLVTVSR |
| SEQ ID NO: 6186 | CD16VL | VL that binds CD16 | SSELTQDPAVSVALGQTVRITCQGDSLRSYYASWYQQ KPGQAPVLVIYGKNNRPSGIPDRFSGSSSGNTASLTITG AQAEDEADYYCNSRDSSGNHVVFGGGTKLTVL |

In one embodiment, the NK cell engager is a ligand ofNKp30, e.g., is a B7-6, e.g., comprises the amino acid sequence of:

DLKVEMMAGGTQITPLNDNVTIFCNIFYSQPLNITSMGITWFWKSLTFDKEVKVFEFFGDH QEAFRPGAIVSPWRLKSGDASLRLPGIQLEEAGEYRCEVVVTPLKAQGTVQLEVVASPASRLLLDQV GMKENEDKYMCESSGFYPEAINITWEKQTQKFPHPIEISEDVITGPTIKNMDGTFNVTSCLKLNSSQE DPGTVYQCVVRHASLHTPLRSNFTLTAARHSLSETEKTDNFS (SEQ ID NO: 7233), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7233.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 Sep;31(9):2680-9 “Recognition of viral hemagglutinins by NKp44 but not by NKp30”; and Nature. 2001 Feb 22;409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein:
(i) MICA comprises the amino acid sequence:
   EPHSLRYNLTVLSWDGSVQSGFLTEVHLDGQPFLRCDRQKCRAKPQGQWAEDVLGNKTWDRETR DLTGNGKDLRMTLAHIKDQKEGLHSLQEIRVCEIHEDNSTRSSQHFYYDGELFLSQNLETKEWTMP QSSRAQTLAMNVRNFLKEDAMKTKTHYHAMHADCLQELRRYLKSGVVLRRTVPPMVNVTRSEAS EGNITVTCRASGFYPWNITLSWRQDGVSLSHDTQQWGDVLPDGNGTYQTWVATRICQGEEQRFTC YMEHSGNHSTHPVPSGKVLVLQSHW (SEQ ID NO: 7234), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7234;
(ii) MICB comprises the amino acid sequence:
   AEPHSLRYNLMVLSQDESVQSGFLAEGHLDGQPFLRYDRQKRRAKPQGQWAEDVLGAKTWDTET EDLTENGQDLRRTLTHIKDQKGGLHSLQEIRVCEIHEDSSTRGSRHFYYDGELFLSQNLETQESTVPQ SSRAQTLAMNVTNFWKEDAMKTKTHYRAMQADCLQKLQRYLKSGVAIRRTVPPMVNVTCSEVSE GNITVTCRASSFYPRNITLTWRQDGVSLSHNTQQWGDVLPDGNGTYQTWVATRIRQGEEQRFTCY MEHSGNHGTHPVPSGKVLVLQSQRTD (SEQ ID NO: 7235), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7235; or
(iii) ULBP1 comprises the amino acid sequence:
   GWVDTHCLCYDFIITPKSRPEPQWCEVQGLVDERPFLHYDCVNHKAKAFASLGKKVNVTKTWEEQ TETLRDVVDFLKGQLLDIQVENLIPIEPLTLQARMSCEHEAHGHGRGSWQFLFNGQKFLLFDSNNRK WTALHPGAKKMTEKWEKNRDVTMFFQKISLGDCKMWLEEFLMYWEQMLDPTKPPSLAPG (SEQ ID NO: 7236), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7236.

In other embodiments, the NK cell engager is a ligand of DNAM1 chosen from NECTIN2 or NECL5, e.g., wherein:
(i) NECTIN2 comprises the amino acid sequence:
   QDVRVQVLPEVRGQLGGTVELPCHLLPPVPGLYISLVTWQRPDAPANHQNVAAFHPKMGPSFPSPK PGSERLSFVSAKQSTGQDTEAELQDATLALHGLTVEDEGNYTCEFATFPKGSVRGMTWLRVIAKPK NQAEAQKVTFSQDPTTVALCISKEGRPPARISWLSSLDWEAKETQVSGTLAGTVTVTSRFTLVPSGR ADGVTVTCKVEHESFEEPALIPVTLSVRYPPEVSISGYDDNWYLGRTDATLSCDVRSNPEPTGYDWS TTSGTFPTSAVAQGSQLVIHAVDSLFNTTFVCTVTNAVGMGRAEQVIFVRETPNTAGAGATGG (SEQ ID NO: 7237), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7237; or
(ii) NECL5 comprises the amino acid sequence:
   WPPPGTGDVVVQAPTQVPGFLGDSVTLPCYLQVPNMEVTHVSQLTWARHGESGSMAVFHQTQGPS YSESKRLEFVAARLGAELRNASLRMFGLRVEDEGNYTCLFVTFPQGSRSVDIWLRVLAKPQNTAEV QKVQLTGEPVPMARCVSTGGRPPAQITWHSDLGGMPNTSQVPGFLSGTVTVTSLWILVPSSQVDGK NVTCKVEHESFEKPQLLTVNLTVYYPPEVSISGYDNNWYLGQNEATLTCDARSNPEPTGYNWSTTM GPLPPFAVAQGAQLLIRPVDKPINTTLICNVTNALGARQAELTVQVKEGPPSEHSGISRN (SEQ ID NO: 7238), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7238.

In yet other embodiments, the NK cell engager is a ligand of DAP10, which is an adapter for NKG2D (see e.g., Proc Natl Acad Sci U S A. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD 16,the full contents of which are incorporated herein).

In other embodiments, the NK cell engager is a ligand of CRTAM, which is NECL2, e.g., wherein NECL2 comprises the amino acid sequence:

QNLFTKDVTVIEGEVATISCQVNKSDDSVIQLLNPNRQTIYFRDFRPLKD
SRFQLLNFSSSELKVSLTNVSISDEGRYFCQLYTDPPQESYTTITVLVPP
RNLMIDIQKDTAVEGEEIEVNCTAMASKPATTIRWFKGNTELKGKSEVEE
WSDMYTVTSQLMLKVHKEDDGVPVICQVEHPAVTGNLQTQRYLEVQYKPQ
VHIQMTYPLQGLTREGDALELTCEAIGKPQPVMVTWVRVDDEMPQHAVLS
GPNLFINNLNKTDNGTYRCEASNIVGKAHSDYMLYVYDPPTTIPPPTTTT
TTTTTTTTTILTIITDSRAGEEGSIRAVDH (SEQ ID NO: 7239),

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7239.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQLRIHRDGI YMVHIQVTLAICSSTTASRHHPTTLAVGICSPASRSISLLRLSFHQGCTIASQRLTPLARGDTLCTNLT GTLLPSRNTDETFFGVQWVRP (SEQ ID NO: 7240), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7240.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGIWTWVGTN KSLTEEAENWGDGEPNNKKNKEDCVEIYIKRNKDAGKWNDDACHKLKAALCYTASCQPWSCSGH GECVEIINNYTCNCDVGYYGPQCQFVIQCEPLEAPELGTMDCTHPLGNFSFSSQCAFSCSEGTNLTGI EETTCGPFGNWSSPEPTCQVIQCEPLSAPDLGIMNCSHPLASFSFTSACTFICSEGTELIGKKKTICESS GIWSNPSPICQKLDKSFSMIKEGDYN (SEQ ID NO: 7241), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7241.

In other embodiments, the NK cell engager is a ligand of CD96, which is NECL5, e.g., wherein NECL5 comprises the amino acid sequence:

WPPPGTGDVVVQAPTQVPGFLGDSVTLPCYLQVPNMEVTHVSQLTWARHG
ESGSMAVFHQTQGPSYSESKRLEFVAARLGAELRNASLRMFGLRVEDEGN
YTCLFVTFPQGSRSVDIWLRVLAKPQNTAEVQKVQLTGEPVPMARCVSTG
GRPPAQITWHSDLGGMPNTSQVPGFLSGTVTVTSLWILVPSSQVDGKNVT
CKVEHESFEKPQLLTVNLTVYYPPEVSISGYDNNWYLGQNEATLTCDARS
NPEPTGYNWSTTMGPLPPFAVAQGAQLLIRPVDKPINTTLICNVTNALGA
RQAELTVQVKEGPPSEHSGISRN (SEQ ID NO: 7238),

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence ofSEQ ID NO: 7238.

In other embodiments, the NK cell engager is a ligand of CD100 (SEMA4D), which is CD72, e.g., wherein CD72 comprises the amino acid sequence: RYLQVSQQLQQTNRVLEVTNSSLRQQLRLKITQLGQSAEDLQGSRRELAQSQEALQVEQRAHQAAE GQLQACQADRQKTKETLQSEEQQRRALEQKLSNMENRLKPFFTCGSADTCCPSGWIMHQKSCFYIS LTSKNWQESQKQCETLSSKLATFSEIYPQSHSYYFLNSLLPNGGSGNSYWTGLSSNKDWKLTDDTQ RTRTYAQSSKCNKVHKTWSWWTLESESCRSSLPYICEMTAFRFPD (SEQ ID NO: 7242), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7242.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRRYKCSSDH WIGLKMAKNRTGQWVDGATFTKSFGMRGSEGCAYLSDDGAATARCYTERKWICRKRIH (SEQ ID NO: 7243), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7243.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGRVRLDPQSG ALYISKVQKEDNSTYIMRVLKKTGNEQEWKIKLQVLDPVPKPVIKIEKIEDMDDNCYLKLSCVIPGE SVNYTWYGDKRPFPKELQNSVLETTLMPHNYSRCYTCQVSNSVSSKNGTVCLSPPCTLARS (SEQ ID NO: 7244), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7244.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

### Antibody Molecules

In an embodiment, the anti-NKp30 antibody molecule is a monospecific antibody molecule and binds a single epitope on NKp30. E.g., a monospecific antibody molecule having a plurality of immunoglobulin variable domain sequences, each of which binds the same epitope.

In another embodiment, the anti-NKp30 antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular’s AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246: 1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L.L. et al. 1994 Nature Genet. 7:13-21; Morrison, S.L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 EurJ Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. US 5,585,089, US 5,693,761 and US 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. 5,225,539; Jones et al. 1986 Nature 321:552-525; Verhoeyan et al. 1988 Science 239:1534; Beidler et al. 1988 J. Immunol. 141:4053-4060; Winter US 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter US 5,225,539), the contents of which is expressly incorporated by reference.

Also within the scope of the invention are humanized antibody molecules in which specific amino acids have been substituted, deleted or added. Criteria for selecting amino acids from the donor are described in US 5,585,089, e.g., columns 12-16 of US 5,585,089, e.g., columns 12-16 of US 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFv) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, I11.

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., cancer cell) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C- terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

In embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and US 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, US9359437, US20150018529, WO2016115274A1, WO2016087416A1, US20080069820A1, US9145588B, US7919257, and US20150232560A1. Exemplary multispecific molecules utilizing a full antibody-Fab/scFab format include those described in the following, US9382323B2, US20140072581A1, US20140308285A1, US20130165638A1, US20130267686A1, US20140377269A1, US7741446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, US8703132B2, US20100316645, US8227577B2, US20130078249.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Fc-Containing Multispecific Molecules

In some embodiments, the multispecific molecules disclosed herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-in-Hole

Knob-in-Hole as described in US 5,731,116, US 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., US7642228).

Exemplary KiH mutations include S354C, T366W in the “knob” heavy chain and Y349C, T366S, L368A, Y407V in the “hole” heavy chain. Other exemplary KiH mutations are provided in Table 1, with additional optional stabilizing Fc cysteine mutations.

TABLE 1

Exemplary Fc KiH mutations and optional Cysteine mutations

| Position | Knob Mutation | Hole Mutation |
|---|---|---|
| T366 | T366W | T366S |
| L368 | - | L368A |
| Y407 | - | Y407V |
| Additional Cysteine Mutations to form a stabilizing disulfide bridge | | |
| Position | Knob CH3 | Hole CH3 |
| S354 | S354C | - |
| Y349 | - | Y349C |

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic- Met antibody inhibits glioblastoma growth in vivo. Clin Cancer Res 2006; 12:6144-52; PMID: 17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore GL et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, US7750128B2, US20160229915A1, US20150344570A1, US8003774A1, US20150337049A1, US20150175707A1, US20140242075A1, US20130195849A1, US20120149876A1, US20140200331A1, US9309311B2, US8586713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., US7183076. Other exemplary cysteine modifications include, e.g., those disclosed in US20140348839A1, US7855275B2, and US9000130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis JH et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and US8871912. The contents of each of which are incorporated by reference herein).

### Duobody

“Duobody” technology to produce bispecific antibodies with correct heavy chain pairing are known. The DuoBody technology involves three basic steps to generate stable bispecific human IgG1antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013;110(13):5145-5150 and Labrijn et al. Nature Protocols 2014;9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3—CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, US8592562B2, US9200060B2, US20140154254A1, and US9358286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., US7183076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids nonspecific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017, incorporated herein by reference in its entirety.

In embodiments, the multispecific molecules includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope;
   a heavy chain polypeptide 1 (HCP1) specific for the first epitope;
   a kappa light chain polypeptide 2 (KLCP2) specific for a second epitope; and
   a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiments it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1region. In an embodiments it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:
LLCP1 has a higher affinity for HCP1 than for HCP2; and/or
KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9 % of the multispecific antibody molecule molecules have a LLCP1complexed, or interfaced with, a HCP1.

In some embodiments of the multispecific antibody molecule disclosed herein:
the HCP1 has a greater affinity for HCP2, than for a second molecule of HCP1; and/or
the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9 % of the multispecific antibody molecule molecules have a HCP1complexed, or interfaced with, a HCP2.

In another aspect, disclosed herein is a method for making, or producing, a multispecific antibody molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VL□), a lambda light constant chain (VL□), or both) that preferentially associates with the first heavy chain polypeptide (e.g., the first VH); and
(iv) providing a kappa chain polypeptide (e.g., a lambda light variable region (VL□), a lambda light constant chain (VL□), or both) that preferentially associates with the second heavy chain polypeptide (e.g., the second VH),
under conditions where (i)-(iv) associate.

In embodiments, the first and second heavy chain polypeptides form an Fc interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiment, the method further comprises purifying a cell-expressed antibody molecule, e.g., using a lambda- and/or- kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75%, 80, 90, 95, 98, 99 99.5 or 99.9 %.

In other embodiments, the multispecific, e.g., a bispecific, antibody molecule that includes:
(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLl), a lambda light constant chain (VLl), or both) that preferentially associates with the first heavy chain polypeptide (e.g., the first VH), e.g., wherein the LLCP1 binds to a first epitope; and
(iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In embodiments, the first and second heavy chain polypeptides form an Fc interface that enhances heterodimerization. In embodiments, the multispecific antibody molecule has a first binding specificity that includes a hybrid VLl-CLl heterodimerized to a first heavy chain variable region connected to the Fc constant, CH2—CH3 domain (having a knob modification) and a second binding specificity that includes a hybrid VLk-CLk heterodimerized to a second heavy chain variable region connected to the Fc constant, CH2—CH3 domain (having a hole modification).

### Linkers

The multispecific or multifunctional molecule disclosed herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In embodiments, the linker is chosen from: a cleavable linker, a non-cleavable linker, a peptide linker, a flexible linker, a rigid linker, a helical linker, or a non-helical linker, or a combination thereof.

In one embodiment, the multispecific molecule can include one, two, three or four linkers, e.g., a peptide linker. In one embodiment, the peptide linker includes Gly and Ser. In some embodiments, the peptide linker is selected from GGGGS (SEQ ID NO: 42); GGGGSGGGGS (SEQ ID NO: 43); GGGGSGGGGSGGGGS (SEQ ID NO: 44); and DVPSGPGGGGGSGGGGS (SEQ ID NO: 45). In some embodiments, the peptide linker is a A(EAAAK)nA (SEQ ID NO: 6154) family of linkers (e.g., as described in Protein Eng. (2001) 14 (8): 529-532). These are stiff helical linkers with n ranging from 2 - 5. In some embodiments, the peptide linker is selected from AEAAAKEAAAKAAA (SEQ ID NO: 75); AEAAAKEAAAKEAAAKAAA (SEQ ID NO: 76); AEAAAKEAAAKEAAAKEAAAKAAA (SEQ ID NO: 77); and AEAAAKEAAAKEAAAKEAAAKEAAAKAAA(SEQ ID NO: 78).

### Targeting Moieties

In one embodiment, the anti-NKp30 antibody molecule further comprises a second antigen binding moiety, e.g., tumor targeting moiety, that binds to a cancer antigen, e.g., a tumor antigen or a stromal antigen. In some embodiments, the cancer antigen is, e.g., a mammalian, e.g., a human, cancer antigen. In other embodiments, the antibody molecule further comprises a second binding moiety that binds to an immune cell antigen, e.g., a mammalian, e.g., a human, immune cell antigen. In other embodiments, the antibody molecule further comprises a second binding moiety that binds to a viral antigen. For example, the antibody molecule binds specifically to an epitope, e.g., linear or conformational epitope, on the cancer antigen, the immune cell antigen.

In some embodiments, the multispecific (e.g., bi-, tri-, tetra- specific) molecule, includes, e.g., is engineered to contain, one or more tumor specific targeting moieties that direct the molecule to a tumor cell. In certain embodiments, the multispecific molecules disclosed herein include a tumor-targeting moiety. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multispecific molecules disclosed herein to a cancer (e.g., a cancer or tumor cells). In some embodiments, the cancer is chosen from a hematological cancer, a solid cancer, a metastatic cancer, or a combination thereof.

In some embodiments, the multispecific molecule, e.g., the tumor-targeting moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In one embodiment, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium.

In certain embodiments, the solid tumor antigen is chosen from one or more of: PDL1, CD47, mesothelin, ganglioside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA½, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, CD47, α actinin-4, TRP1/gp75, TRP2, gp100, Melan-A/MART1, gangliosides, WT1, EphA3, Epidermal growth factor receptor (EGFR), CD20, MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL. In some embodiments, the solid tumor antigen is chosen from: PDL1, Mesothelin, CD47, GD2, PMSA, PSCA, CEA, Ron Kinase, or c-Met. Exemplary amino acid and nucleotide sequences for tumor targeting moieties are disclosed in WO 2017/165464, see e.g., pages 102-108, 172-290, incorporated herein by reference.

In some embodiments, the anti-NKp30 antibody molecule (e.g., the multispecific antibody molecule) further comprises a targeting moiety, e.g., a binding specificity, that binds to an autoreactive T cell, e.g., an antigen present on the surface of an autoreactive T cell that is associated with the inflammatory or autoimmune disorder.

In some embodiments, the anti-NKp30 antibody molecule (e.g., the multispecific antibody molecule) further comprises a targeting moiety, e.g., a binding specificity, that binds to an infected cell, e.g., a viral infected cell.

### T Cell Engagers

In other embodiments, the anti-NKp30 antibody molecule (e.g., the multispecific antibody molecule) further comprises one or more T cell engager that mediate binding to and/or activation of a T cell. Accordingly, in some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226.

Exemplary T cell engagers are disclosed in WO 2017/165464, incorporated herein by reference.

### Cytokine Molecules

In other embodiments, the anti-NKp30 antibody molecule (e.g., the multispecific antibody molecule) further comprises one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In one embodiment the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In one embodiment the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In one embodiment, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor. Together with the beta- and gamma-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In one embodiment, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In one embodiment the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In one embodiment the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In one embodiment said additional amino acid mutation is the amino acid substitution C125A.

Exemplary cytokine molecules are disclosed in WO 2017/165464, see e.g., pages 108-118, 169-172, incorporated herein by reference.

### TGF-β Inhibitor

In other embodiments, the anti-NKp30 antibody molecule (e.g., the multispecific antibody molecule) further comprises one or more modulators of TGF-β (e.g., a TGF-β inhibitor). In some embodiments, the TGF-β inhibitor binds to and inhibits TGF-β, e.g., reduces the activity of TGF-β. In some embodiments, the TGF-β inhibitor inhibits (e.g., reduces the activity of) TGF-β 1. In some embodiments, the TGF-β inhibitor inhibits (e.g., reduces the activity of) TGF-β 2. In some embodiments, the TGF-β inhibitor inhibits (e.g., reduces the activity of) TGF-β 3. In some embodiments, the TGF-β inhibitor inhibits (e.g., reduces the activity of) TGF-β 1 and TGF-β 3. In some embodiments, the TGF-β inhibitor inhibits (e.g., reduces the activity of) TGF-β 1, TGF-β 2, and TGF-β 3.

In some embodiments, the TGF-β inhibitor comprises a portion of a TGF-β receptor (e.g., an extracellular domain of a TGF-β receptor) that is capable of inhibiting (e.g., reducing the activity of) TGF-β, or functional fragment or variant thereof. In some embodiments, the TGF-β inhibitor comprises a TGFBR1 polypeptide (e.g., an extracellular domain of TGFBR1 or functional variant thereof). In some embodiments, the TGF-β inhibitor comprises a TGFBR2 polypeptide (e.g., an extracellular domain of TGFBR2 or functional variant thereof). In some embodiments, the TGF-β inhibitor comprises a TGFBR3 polypeptide (e.g., an extracellular domain of TGFBR3 or functional variant thereof). In some embodiments, the TGF-β inhibitor comprises a TGFBR1 polypeptide (e.g., an extracellular domain of TGFBR1 or functional variant thereof) and a TGFBR2 polypeptide (e.g., an extracellular domain of TGFBR2 or functional variant thereof). In some embodiments, the TGF-β inhibitor comprises a TGFBR1 polypeptide (e.g., an extracellular domain of TGFBR1 or functional variant thereof) and a TGFBR3 polypeptide (e.g., an extracellular domain of TGFBR3 or functional variant thereof). In some embodiments, the TGF-β inhibitor comprises a TGFBR2 polypeptide (e.g., an extracellular domain of TGFBR2 or functional variant thereof) and a TGFBR3 polypeptide (e.g., an extracellular domain of TGFBR3 or functional variant thereof).

Exemplary TGF-β receptor polypeptides that can be used as TGF-β inhibitors have been disclosed in US8993524, US9676863, US8658135, US20150056199, US20070184052, and WO2017037634, all of which are herein incorporated by reference in their entirety.

In some embodiments, the TGF-β inhibitor comprises an extracellular domain of TGFBR1 or a sequence substantially identical thereto (e.g., a sequence that is at least 80%, 85%, 90%, or 95% identical thereto). In some embodiments, the TGF-β inhibitor comprises an extracellular domain of SEQ ID NO: 3095, or a sequence substantially identical thereto (e.g., a sequence that is at least 80%, 85%, 90%, or 95% identical thereto). In some embodiments, the TGF-β inhibitor comprises an extracellular domain of SEQ ID NO: 3096, or a sequence substantially identical thereto (e.g., a sequence that is at least 80%, 85%, 90%, or 95% identical thereto). In some embodiments, the TGF-β inhibitor comprises an extracellular domain of SEQ ID NO: 3097, or a sequence substantially identical thereto (e.g., a sequence that is at least 80%, 85%, 90%, or 95% identical thereto). In some embodiments, the TGF-β inhibitor comprises the amino acid sequence of SEQ ID NO: 3104, or a sequence substantially identical thereto (e.g., a sequence that is at least 80%, 85%, 90%, or 95% identical thereto). In some embodiments, the TGF-β inhibitor comprises the amino acid sequence of SEQ ID NO: 3105, or a sequence substantially identical thereto (e.g., a sequence that is at least 80%, 85%, 90%, or 95% identical thereto).

In some embodiments, the TGF-β inhibitor comprises an extracellular domain of TGFBR2 or a sequence substantially identical thereto (e.g., a sequence that is at least 80%, 85%, 90%, or 95% identical thereto). In some embodiments, the TGF-β inhibitor comprises an extracellular domain of SEQ ID NO: 3098, or a sequence substantially identical thereto (e.g., a sequence that is at least 80%, 85%, 90%, or 95% identical thereto). In some embodiments, the TGF-β inhibitor comprises an extracellular domain of SEQ ID NO: 3099, or a sequence substantially identical thereto (e.g., a sequence that is at least 80%, 85%, 90%, or 95% identical thereto). In some embodiments, the TGF-β inhibitor comprises the amino acid sequence of SEQ ID NO: 3100, or a sequence substantially identical thereto (e.g., a sequence that is at least 80%, 85%, 90%, or 95% identical thereto). In some embodiments, the TGF-β inhibitor comprises the amino acid sequence of SEQ ID NO: 3101, or a sequence substantially identical thereto (e.g., a sequence that is at least 80%, 85%, 90%, or 95% identical thereto). In some embodiments, the TGF-β inhibitor comprises the amino acid sequence of SEQ ID NO: 3102, or a sequence substantially identical thereto (e.g., a sequence that is at least 80%, 85%, 90%, or 95% identical thereto). In some embodiments, the TGF-β inhibitor comprises the amino acid sequence of SEQ ID NO: 3103, or a sequence substantially identical thereto (e.g., a sequence that is at least 80%, 85%, 90%, or 95% identical thereto).

In some embodiments, the TGF-β inhibitor comprises an extracellular domain of TGFBR3 or a sequence substantially identical thereto (e.g., a sequence that is at least 80%, 85%, 90%, or 95% identical thereto). In some embodiments, the TGF-β inhibitor comprises an extracellular domain of SEQ ID NO: 3106, or a sequence substantially identical thereto (e.g., a sequence that is at least 80%, 85%, 90%, or 95% identical thereto). In some embodiments, the TGF-β inhibitor comprises an extracellular domain of SEQ ID NO: 3107, or a sequence substantially identical thereto (e.g., a sequence that is at least 80%, 85%, 90%, or 95% identical thereto). In some embodiments, the TGF-β inhibitor comprises the amino acid sequence of SEQ ID NO: 3108, or a sequence substantially identical thereto (e.g., a sequence that is at least 80%, 85%, 90%, or 95% identical thereto).

In some embodiments, the TGF-β inhibitor comprises no more than one TGF-β receptor extracellular domain. In some embodiments, the TGF-β inhibitor comprises two or more (e.g., two, three, four, five, or more) TGF-β receptor extracellular domains, linked together, e.g., via a linker.

TABLE 4

Exemplary amino acid sequences of TGF-β polypeptides or TGF-β receptor polypeptides

| | | |
|---|---|---|
| SEQ ID NO | Description | Amino acid sequence |
| SEQ ID NO: 3092 | Immature human TGF-β 1 (P01137-1) | MPPSGLRLLLLLLPLLWLLVLTPGRPAAGLSTCKTIDMELVKRK RIEAIRGQILSKLRLASPPSQGEVPPGPLPEAVLALYNSTRDRVA GESAEPEPEPEADYYAKEVTRVLMVETHNEIYDKFKQSTHSIYM FFNTSELREAVPEPVLLSRAELRLLRLKLKVEQHVELYQKYSNN SWRYLSNRLLAPSDSPEWLSFDVTGVVRQWLSRGGEIEGFRLSA HCSCDSRDNTLQVDINGFTTGRRGDLATIHGMNRPFLLLMATPL ERAQHLQSSRHRRALDTNYCFSSTEKNCCVRQLYIDFRKDLGW KWIHEPKGYHANFCLGPCPYIWSLDTQYSKVLALYNQHNPGAS AAPCCVPQALEPLPIVYYVGRKPKVEQLSNMIVRSCKCS |
| SEQ ID NO: 3117 | Human TGF-β 1 (P01137-1) | LSTCKTIDMELVKRKRIEAIRGQILSKLRLASPPSQGEVPPGPLPE AVLALYNSTRDRVAGESAEPEPEPEADYYAKEVTRVLMVETHN EIYDKFKQSTHSIYMFFNTSELREAVPEPVLLSRAELRLLRLKLK VEQHVELYQKYSNNSWRYLSNRLLAPSDSPEWLSFDVTGVVRQ WLSRGGEIEGFRLSAHCSCDSRDNTLQVDINGFTTGRRGDLATI HGMNRPFLLLMATPLERAQHLQSSRHRRALDTNYCFSSTEKNC CVRQLYIDFRKDLGWKWIHEPKGYHANFCLGPCPYIWSLDTQY SKVLALYNQHNPGASAAPCCVPQALEPLPIVYYVGRKPKVEQL SNMIVRSCKCS |
| SEQ ID NO: 3093 | Immature human TGF-β 2 (P61812-1) | MHYCVLSAFLILHLVTVALSLSTCSTLDMDQFMRKRIEAIRGQIL SKLKLTSPPEDYPEPEEVPPEVISIYNSTRDLLQEKASRRAAACER ERSDEEYYAKEVYKIDMPPFFPSENAIPPTFYRPYFRIVRFDVSA |
| | | MEKNASNLVKAEFRVFRLQNPKARVPEQRIELYQILKSKDLTSP TQRYIDSKVVKTRAEGEWLSFDVTDAVHEWLHHKDRNLGFKIS LHCPCCTFVPSNNYIIPNKSEELEARFAGIDGTSTYTSGDQKTIKS TRKKNSGKTPHLLLMLLPSYRLESQQTNRRKKRALDAAYCFRN VQDNCCLRPLYIDFKRDLGWKWIHEPKGYNANFCAGACPYLW SSDTQHSRVLSLYNTINPEASASPCCVSQDLEPLTILYYIGKTPKI EQLSNMIVKSCKCS |
| SEQ ID NO: 3118 | Human TGF-β 2 (P61812-1) | LSTCSTLDMDQFMRKRIEAIRGQILSKLKLTSPPEDYPEPEEVPPE VISIYNSTRDLLQEKASRRAAACERERSDEEYYAKEVYKIDMPP FFPSENAIPPTFYRPYFRIVRFDVSAMEKNASNLVKAEFRVFRLQ NPKARVPEQRIELYQILKSKDLTSPTQRYIDSKVVKTRAEGEWL SFDVTDAVHEWLHHKDRNLGFKISLHCPCCTFVPSNNYIIPNKSE ELEARFAGIDGTSTYTSGDQKTIKSTRKKNSGKTPHLLLMLLPSY RLESQQTNRRKKRALDAAYCFRNVQDNCCLRPLYIDFKRDLGW KWIHEPKGYNANFCAGACPYLWSSDTQHSRVLSLYNTINPEAS ASPCCVSQDLEPLTILYYIGKTPKIEQLSNMIVKSCKCS |
| SEQ ID NO: 3094 | Immature human TGF-β 3 (P10600-1) | MKMHLQRALVVLALLNFATVSLSLSTCTTLDFGHIKKKRVEAIR GQILSKLRLTSPPEPTVMTHVPYQVLALYNSTRELLEEMHGERE EGCTQENTESEYYAKEIHKFDMIQGLAEHNELAVCPKGITSKVF RFNVSSVEKNRTNLFRAEFRVLRVPNPSSKRNEQRIELFQILRPD EHIAKQRYIGGKNLPTRGTAEWLSFDVTDTVREWLLRRESNLG LEISIHCPCHTFQPNGDILENIHEVMEIKFKGVDNEDDHGRGDLG RLKKQKDHHNPHLILMMIPPHRLDNPGQGGQRKKRALDTNYCF RNLEENCCVRPLYIDFRQDLGWKWVHEPKGYYANFCSGPCPYL RSADTTHSTVLGLYNTLNPEASASPCCVPQDLEPLTILYYVGRTP KVEQLSNMVVKSCKCS |
| SEQ ID NO: 3119 | Human TGF-β 3 (P10600-1) | LSTCTTLDFGHIKKKRVEAIRGQILSKLRLTSPPEPTVMTHVPYQ VLALYNSTRELLEEMHGEREEGCTQENTESEYYAKEIHKFDMIQ GLAEHNELAVCPKGITSKVFRFNVSSVEKNRTNLFRAEFRVLRV PNPSSKRNEQRIELFQILRPDEHIAKQRYIGGKNLPTRGTAEWLS FDVTDTVREWLLRRESNLGLEISIHCPCHTFQPNGDILENIHEVM EIKFKGVDNEDDHGRGDLGRLKKQKDHHNPHLILMMIPPHRLD NPGQGGQRKKRALDTNYCFRNLEENCCVRPLYIDFRQDLGWK WVHEPKGYYANFCSGPCPYLRSADTTHSTVLGLYNTLNPEASA SPCCVPQDLEPLTILYYVGRTPKVEQLSNMVVKSCKCS |
| SEQ ID NO: 3095 | Immature human TGFBR1 isoform 1 (P36897-1) | MEAAVAAPRPRLLLLVLAAAAAAAAALLPGATALQCFCHLCT KDNFTCVTDGLCFVSVTETTDKVIHNSMCIAEIDLIPRDRPFVCA PSSKTGSVTTTYCCNQDHCNKIELPTTVKSSPGLGPVELAAVIAG PVCFVCISLMLMVYICHNRTVIHHRVPNEEDPSLDRPFISEGTTL KDLIYDMTTSGSGSGLPLLVQRTIARTIVLQESIGKGRFGEVWRG KWRGEEVAVKIFSSREERSWFREAEIYQTVMLRHENILGFIAAD NKDNGTWTQLWLVSDYHEHGSLFDYLNRYTVTVEGMIKLALS TASGLAHLHMEIVGTQGKPAIAHRDLKSKNILVKKNGTCCIADL GLAVRHDSATDTIDIAPNHRVGTKRYMAPEVLDDSINMKHFESF KRADIYAMGLVFWEIARRCSIGGIHEDYQLPYYDLVPSDPSVEE MRKVVCEQKLRPNIPNRWQSCEALRVMAKIMRECWYANGAA RLTALRIKKTLSQLSQQEGIKM |
| SEQ ID NO: 3120 | Human TGFBR1 isoform 1 (P36897-1) | LQCFCHLCTKDNFTCVTDGLCFVSVTETTDKVIHNSMCIAEIDLI PRDRPFVCAPSSKTGSVTTTYCCNQDHCNKIELPTTVKSSPGLGP VELAAVIAGPVCFVCISLMLMVYICHNRTVIHHRVPNEEDPSLD RPFISEGTTLKDLIYDMTTSGSGSGLPLLVQRTIARTIVLQESIGK GRFGEVWRGKWRGEEVAVKIFSSREERSWFREAEIYQTVMLRH ENILGFIAADNKDNGTWTQLWLVSDYHEHGSLFDYLNRYTVTV |
| | | EGMIKLALSTASGLAHLHMEIVGTQGKPAIAHRDLKSKNILVKK NGTCCIADLGLAVRHDSATDTIDIAPNHRVGTKRYMAPEVLDD SINMKHFESFKRADIYAMGLVFWEIARRCSIGGIHEDYQLPYYD LVPSDPSVEEMRKVVCEQKLRPNIPNRWQSCEALRVMAKIMRE CWYANGAARLTALRIKKTLSQLSQQEGIKM |
| SEQ ID NO: 3096 | Immature human TGFBR1 isoform 2 (P36897-2) | MEAAVAAPRPRLLLLVLAAAAAAAAALLPGATALQCFCHLCT KDNFTCVTDGLCFVSVTETTDKVIHNSMCIAEIDLIPRDRPFVCA PSSKTGSVTTTYCCNQDHCNKIELPTTGPFSVKSSPGLGPVELAA VIAGPVCFVCISLMLMVYICHNRTVIHHRVPNEEDPSLDRPFISE GTTLKDLIYDMTTSGSGSGLPLLVQRTIARTIVLQESIGKGRFGE VWRGKWRGEEVAVKIFSSREERSWFREAEIYQTVMLRHENILG FIAADNKDNGTWTQLWLVSDYHEHGSLFDYLNRYTVTVEGMI KLALSTASGLAHLHMEIVGTQGKPAIAHRDLKSKNILVKKNGTC CIADLGLAVRHDSATDTIDIAPNHRVGTKRYMAPEVLDDSINMK HFESFKRADIYAMGLVFWEIARRCSIGGIHEDYQLPYYDLVPSD PSVEEMRKVVCEQKLRPNIPNRWQSCEALRVMAKIMRECWYA NGAARLTALRIKKTLSQLSQQEGIKM |
| SEQ ID NO: 3121 | Human TGFBR1 isoform 2 (P36897-2) | LQCFCHLCTKDNFTCVTDGLCFVSVTETTDKVIHNSMCIAEIDLI PRDRPFVCAPSSKTGSVTTTYCCNQDHCNKIELPTTGPFSVKSSP GLGPVELAAVIAGPVCFVCISLMLMVYICHNRTVIHHRVPNEED PSLDRPFISEGTTLKDLIYDMTTSGSGSGLPLLVQRTIARTIVLQE SIGKGRFGEVWRGKWRGEEVAVKIFSSREERSWFREAEIYQTV MLRHENILGFIAADNKDNGTWTQLWLVSDYHEHGSLFDYLNR YTVTVEGMIKLALSTASGLAHLHMEIVGTQGKPAIAHRDLKSK NILVKKNGTCCIADLGLAVRHDSATDTIDIAPNHRVGTKRYMAP EVLDDSINMKHFESFKRADIYAMGLVFWEIARRCSIGGIHEDYQ LPYYDLVPSDPSVEEMRKVVCEQKLRPNIPNRWQSCEALRVMA KIMRECWYANGAARLTALRIKKTLSQLSQQEGIKM |
| SEQ ID NO: 3097 | Immature human TGFBR1 isoform 3 (P36897-3) | MEAAVAAPRPRLLLLVLAAAAAAAAALLPGATALQCFCHLCT KDNFTCVTDGLCFVSVTETTDKVIHNSMCIAEIDLIPRDRPFVCA PSSKTGSVTTTYCCNQDHCNKIELPTTGLPLLVQRTIARTIVLQE SIGKGRFGEVWRGKWRGEEVAVKIFSSREERSWFREAEIYQTV MLRHENILGFIAADNKDNGTWTQLWLVSDYHEHGSLFDYLNR YTVTVEGMIKLALSTASGLAHLHMEIVGTQGKPAIAHRDLKSK NILVKKNGTCCIADLGLAVRHDSATDTIDIAPNHRVGTKRYMAP EVLDDSINMKHFESFKRADIYAMGLVFWEIARRCSIGGIHEDYQ LPYYDLVPSDPSVEEMRKVVCEQKLRPNIPNRWQSCEALRVMA KIMRECWYANGAARLTALRIKKTLSQLSQQEGIKM |
| SEQ ID NO: 3122 | Human TGFBR1 isoform 3 (P36897-3) | LQCFCHLCTKDNFTCVTDGLCFVSVTETTDKVIHNSMCIAEIDLI PRDRPFVCAPSSKTGSVTTTYCCNQDHCNKIELPTTGLPLLVQRT IARTIVLQESIGKGRFGEVWRGKWRGEEVAVKIFSSREERSWFR EAEIYQTVMLRHENILGFIAADNKDNGTWTQLWLVSDYHEHGS LFDYLNRYTVTVEGMIKLALSTASGLAHLHMEIVGTQGKPAIAH RDLKSKNILVKKNGTCCIADLGLAVRHDSATDTIDIAPNHRVGT KRYMAPEVLDDSINMKHFESFKRADIYAMGLVFWEIARRCSIG GIHEDYQLPYYDLVPSDPSVEEMRKVVCEQKLRPNIPNRWQSCE ALRVMAKIMRECWYANGAARLTALRIKKTLSQLSQQEGIKM |
| SEQ ID NO: 3104 | Human TGFBR1 fragment 1 | LQCFCHLCTKDNFTCVTDGLCFVSVTETTDKVIHNSMCIAEIDLI PRDRPFVCAPSSKTGSVTTTYCCNQDHCNKIELPTTVKSSPGLGP VEL |
| SEQ ID NO: 3105 | Human TGFBR1 fragment 2 | ALQCFCHLCTKDNFTCVTDGLCFVSVTETTDKVIHNSMCIAEID LIPRDRPFVCAPSSKTGSVTTTYCCNQDHCNKIEL |
| SEQ ID NO: 3098 | Immature human TGFBR2 isoform B (short isoform) (P37173-1) | MGRGLLRGLWPLHIVLWTRIASTIPPHVQKSVNNDMIVTDNNG AVKFPQLCKFCDVRFSTCDNQKSCMSNCSITSICEKPQEVCVAV WRKNDENITLETVCHDPKLPYHDFILEDAASPKCIMKEKKKPGE TFFMCSCSSDECNDNIIFSEEYNTSNPDLLLVIFQVTGISLLPPLGV AISVIIIFYCYRVNRQQKLSSTWETGKTRKLMEFSEHCAIILEDDR SDISSTCANNINHNTELLPIELDTLVGKGRFAEVYKAKLKQNTSE QFETVAVKIFPYEEYASWKTEKDIFSDINLKHENILQFLTAEERK TELGKQYWLITAFHAKGNLQEYLTRHVISWEDLRKLGSSLARGI AHLHSDHTPCGRPKMPIVHRDLKSSNILVKNDLTCCLCDFGLSL RLDPTLSVDDLANSGQVGTARYMAPEVLESRMNLENVESFKQT DVYSMALVLWEMTSRCNAVGEVKDYEPPFGSKVREHPCVESM KDNVLRDRGRPEIPSFWLNHQGIQMVCETLTECWDHDPEARLT AQCVAERFSELEHLDRLSGRSCSEEKIPEDGSLNTTK |
| SEQ ID NO: 3123 | Human TGFBR2 isoform B (short isoform) (P37173-1) | TIPPHVQKSVNNDMIVTDNNGAVKFPQLCKFCDVRFSTCDNQK SCMSNCSITSICEKPQEVCVAVWRKNDENITLETVCHDPKLPYH DFILEDAASPKCIMKEKKKPGETFFMCSCSSDECNDNIIFSEEYN TSNPDLLLVIFQVTGISLLPPLGVAISVIIIFYCYRVNRQQKLSSTW ETGKTRKLMEFSEHCAIILEDDRSDISSTCANNINHNTELLPIELD TLVGKGRFAEVYKAKLKQNTSEQFETVAVKIFPYEEYASWKTE KDIFSDINLKHENILQFLTAEERKTELGKQYWLITAFHAKGNLQE YLTRHVISWEDLRKLGSSLARGIAHLHSDHTPCGRPKMPIVHRD LKSSNILVKNDLTCCLCDFGLSLRLDPTLSVDDLANSGQVGTAR YMAPEVLESRMNLENVESFKQTDVYSMALVLWEMTSRCNAVG EVKDYEPPFGSKVREHPCVESMKDNVLRDRGRPEIPSFWLNHQ GIQMVCETLTECWDHDPEARLTAQCVAERFSELEHLDRLSGRS CSEEKIPEDGSLNTTK |
| SEQ ID NO: 3099 | Immature human TGFBR2 isoform A (long isoform) (P37173-2) | MGRGLLRGLWPLHIVLWTRIASTIPPHVQKSDVEMEAQKDEIIC PSCNRTAHPLRHINNDMIVTDNNGAVKFPQLCKFCDVRFSTCDN QKSCMSNCSITSICEKPQEVCVAVWRKNDENITLETVCHDPKLP YHDFILEDAASPKCIMKEKKKPGETFFMCSCSSDECNDNIIFSEE YNTSNPDLLLVIFQVTGISLLPPLGVAISVIIIFYCYRVNRQQKLSS TWETGKTRKLMEFSEHCAIILEDDRSDISSTCANNINHNTELLPIE LDTLVGKGRFAEVYKAKLKQNTSEQFETVAVKIFPYEEYASWK TEKDIFSDINLKHENILQFLTAEERKTELGKQYWLITAFHAKGNL QEYLTRHVISWEDLRKLGSSLARGIAHLHSDHTPCGRPKMPIVH RDLKSSNILVKNDLTCCLCDFGLSLRLDPTLSVDDLANSGQVGT ARYMAPEVLESRMNLENVESFKQTDVYSMALVLWEMTSRCNA VGEVKDYEPPFGSKVREHPCVESMKDNVLRDRGRPEIPSFWLN HQGIQMVCETLTECWDHDPEARLTAQCVAERFSELEHLDRLSG RSCSEEKIPEDGSLNTTK |
| SEQ ID NO: 3124 | Human TGFBR2 isoform A (long isoform) (P37173-2) | TIPPHVQKSDVEMEAQKDEIICPSCNRTAHPLRHINNDMIVTDNN GAVKFPQLCKFCDVRFSTCDNQKSCMSNCSITSICEKPQEVCVA VWRKNDENITLETVCHDPKLPYHDFILEDAASPKCIMKEKKKPG ETFFMCSCSSDECNDNIIFSEEYNTSNPDLLLVIFQVTGISLLPPLG VAISVIIIFYCYRVNRQQKLSSTWETGKTRKLMEFSEHCAIILEDD RSDISSTCANNINHNTELLPIELDTLVGKGRFAEVYKAKLKQNTS EQFETVAVKIFPYEEYASWKTEKDIFSDINLKHENILQFLTAEER KTELGKQYWLITAFHAKGNLQEYLTRHVISWEDLRKLGSSLAR GIAHLHSDHTPCGRPKMPIVHRDLKSSNILVKNDLTCCLCDFGL SLRLDPTLSVDDLANSGQVGTARYMAPEVLESRMNLENVESFK QTDVYSMALVLWEMTSRCNAVGEVKDYEPPFGSKVREHPCVE SMKDNVLRDRGRPEIPSFWLNHQGIQMVCETLTECWDHDPEAR LTAQCVAERFSELEHLDRLSGRSCSEEKIPEDGSLNTTK |
| SEQ ID NO: 3100 | Human TGFBR2 fragment 1 (ECD of human TGFBR2 isoform B) | TIPPHVQKSVNNDMIVTDNNGAVKFPQLCKFCDVRFSTCDNQK SCMSNCSITSICEKPQEVCVAVWRKNDENITLETVCHDPKLPYH DFILEDAASPKCIMKEKKKPGETFFMCSCSSDECNDNIIFSEEYN TSNPD |
| SEQ ID NO: 3101 | Human TGFBR2 fragment 2 | IPPHVQKSVNNDMIVTDNNGAVKFPQLCKFCDVRFSTCDNQKS CMSNCSITSICEKPQEVCVAVWRKNDENITLETVCHDPKLPYHD FILEDAASPKCIMKEKKKPGETFFMCSCSSDECNDNIIFSEEYNTS NPD |
| SEQ ID NO: 3102 | Human TGFBR2 fragment 3 (ECD of human TGFBR2 isoform A) | TIPPHVQKSDVEMEAQKDEIICPSCNRTAHPLRHINNDMIVTDNN GAVKFPQLCKFCDVRFSTCDNQKSCMSNCSITSICEKPQEVCVA VWRKNDENITLETVCHDPKLPYHDFILEDAASPKCIMKEKKKPG ETFFMCSCSSDECNDNIIFSEEYNTSNPD |
| SEQ ID NO: 3103 | Human TGFBR2 fragment 4 | QLCKFCDVRFSTCDNQKSCMSNCSITSICEKPQEVCVAVWRKN DENITLETVCHDPKLPYHDFILEDAASPKCIMKEKKKPGETFFM CSCSSDECNDNIIF |
| SEQ ID NO: 3106 | Immature human TGFBR3 isoform 1 (Q03167-1) | MTSHYVIAIFALMSSCLATAGPEPGALCELSPVSASHPVQALME SFTVLSGCASRGTTGLPQEVHVLNLRTAGQGPGQLQREVTLHL NPISSVHIHHKSVVFLLNSPHPLVWHLKTERLATGVSRLFLVSEG SVVQFSSANFSLTAETEERNFPHGNEHLLNWARKEYGAVTSFTE LKIARNIYIKVGEDQVFPPKCNIGKNFLSLNYLAEYLQPKAAEGC VMSSQPQNEEVHIIELITPNSNPYSAFQVDITIDIRPSQEDLEVVK NLILILKCKKSVNWVIKSFDVKGSLKIIAPNSIGFGKESERSMTM TKSIRDDIPSTQGNLVKWALDNGYSPITSYTMAPVANRFHLRLE NNAEEMGDEEVHTIPPELRILLDPGALPALQNPPIRGGEGQNGG LPFPFPDISRRVWNEEGEDGLPRPKDPVIPSIQLFPGLREPEEVQG SVDIALSVKCDNEKMIVAVEKDSFQASGYSGMDVTLLDPTCKA KMNGTHFVLESPLNGCGTRPRWSALDGVVYYNSIVIQVPALGD SSGWPDGYEDLESGDNGFPGDMDEGDASLFTRPEIVVFNCSLQ QVRNPSSFQEQPHGNITFNMELYNTDLFLVPSQGVFSVPENGHV YVEVSVTKAEQELGFAIQTCFISPYSNPDRMSHYTIIENICPKDES VKFYSPKRVHFPIPQADMDKKRFSFVFKPVFNTSLLFLQCELTLC TKMEKHPQKLPKCVPPDEACTSLDASIIWAMMQNKKTFTKPLA VIHHEAESKEKGPSMKEPNPISPPIFHGLDTLTVMGIAFAAFVIGA LLTGALWYIYSHTGETAGRQQVPTSPPASENSSAAHSIGSTQSTP CSSSSTA |
| SEQ ID NO: 3125 | Human TGFBR3 isoform 1 (Q03167-1) | GPEPGALCELSPVSASHPVQALMESFTVLSGCASRGTTGLPQEV HVLNLRTAGQGPGQLQREVTLHLNPISSVHIHHKSVVFLLNSPH PLVWHLKTERLATGVSRLFLVSEGSVVQFSSANFSLTAETEERN FPHGNEHLLNWARKEYGAVTSFTELKIARNIYIKVGEDQVFPPK CNIGKNFLSLNYLAEYLQPKAAEGCVMSSQPQNEEVHIIELITPN SNPYSAFQVDITIDIRPSQEDLEVVKNLILILKCKKSVNWVIKSFD VKGSLKIIAPNSIGFGKESERSMTMTKSIRDDIPSTQGNLVKWAL DNGYSPITSYTMAPVANRFHLRLENNAEEMGDEEVHTIPPELRIL LDPGALPALQNPPIRGGEGQNGGLPFPFPDISRRVWNEEGEDGL PRPKDPVIPSIQLFPGLREPEEVQGSVDIALSVKCDNEKMIVAVE KDSFQASGYSGMDVTLLDPTCKAKMNGTHFVLESPLNGCGTRP RWSALDGVVYYNSIVIQVPALGDSSGWPDGYEDLESGDNGFPG DMDEGDASLFTRPEIVVFNCSLQQVRNPSSFQEQPHGNITFNME LYNTDLFLVPSQGVFSVPENGHVYVEVSVTKAEQELGFAIQTCF ISPYSNPDRMSHYTIIENICPKDESVKFYSPKRVHFPIPQADMDK KRFSFVFKPVFNTSLLFLQCELTLCTKMEKHPQKLPKCVPPDEA |
| | | CTSLDASIIWAMMQNKKTFTKPLAVIHHEAESKEKGPSMKEPNP ISPPIFHGLDTLTVMGIAFAAFVIGALLTGALWYIYSHTGETAGR QQVPTSPPASENSSAAHSIGSTQSTPCSSSSTA |
| SEQ ID NO: 3107 | Immature human TGFBR3 isoform 2 (Q03167-2) | MTSHYVIAIFALMSSCLATAGPEPGALCELSPVSASHPVQALME SFTVLSGCASRGTTGLPQEVHVLNLRTAGQGPGQLQREVTLHL NPISSVHIHHKSVVFLLNSPHPLVWHLKTERLATGVSRLFLVSEG SVVQFSSANFSLTAETEERNFPHGNEHLLNWARKEYGAVTSFTE LKIARNIYIKVGEDQVFPPKCNIGKNFLSLNYLAEYLQPKAAEGC VMSSQPQNEEVHIIELITPNSNPYSAFQVDITIDIRPSQEDLEVVK NLILILKCKKSVNWVIKSFDVKGSLKIIAPNSIGFGKESERSMTM TKSIRDDIPSTQGNLVKWALDNGYSPITSYTMAPVANRFHLRLE NNEEMGDEEVHTIPPELRILLDPGALPALQNPPIRGGEGQNGGLP FPFPDISRRVWNEEGEDGLPRPKDPVIPSIQLFPGLREPEEVQGSV DIALSVKCDNEKMIVAVEKDSFQASGYSGMDVTLLDPTCKAKM NGTHFVLESPLNGCGTRPRWSALDGVVYYNSIVIQVPALGDSSG WPDGYEDLESGDNGFPGDMDEGDASLFTRPEIVVFNCSLQQVR NPSSFQEQPHGNITFNMELYNTDLFLVPSQGVFSVPENGHVYVE VSVTKAEQELGFAIQTCFISPYSNPDRMSHYTIIENICPKDESVKF YSPKRVHFPIPQADMDKKRFSFVFKPVFNTSLLFLQCELTLCTK MEKHPQKLPKCVPPDEACTSLDASIIWAMMQNKKTFTKPLAVI HHEAESKEKGPSMKEPNPISPPIFHGLDTLTVMGIAFAAFVIGAL LTGALWYIYSHTGETAGRQQVPTSPPASENSSAAHSIGSTQSTPC SSSSTA |
| SEQ ID NO: 3126 | Human TGFBR3 isoform 2 (Q03167-2) | GPEPGALCELSPVSASHPVQALMESFTVLSGCASRGTTGLPQEV HVLNLRTAGQGPGQLQREVTLHLNPISSVHIHHKSVVFLLNSPH PLVWHLKTERLATGVSRLFLVSEGSVVQFSSANFSLTAETEERN FPHGNEHLLNWARKEYGAVTSFTELKIARNIYIKVGEDQVFPPK CNIGKNFLSLNYLAEYLQPKAAEGCVMSSQPQNEEVHIIELITPN SNPYSAFQVDITIDIRPSQEDLEVVKNLILILKCKKSVNWVIKSFD VKGSLKIIAPNSIGFGKESERSMTMTKSIRDDIPSTQGNLVKWAL DNGYSPITSYTMAPVANRFHLRLENNEEMGDEEVHTIPPELRILL DPGALPALQNPPIRGGEGQNGGLPFPFPDISRRVWNEEGEDGLP RPKDPVIPSIQLFPGLREPEEVQGSVDIALSVKCDNEKMIVAVEK DSFQASGYSGMDVTLLDPTCKAKMNGTHFVLESPLNGCGTRPR WSALDGVVYYNSIVIQVPALGDSSGWPDGYEDLESGDNGFPGD MDEGDASLFTRPEIVVFNCSLQQVRNPSSFQEQPHGNITFNMEL YNTDLFLVPSQGVFSVPENGHVYVEVSVTKAEQELGFAIQTCFIS PYSNPDRMSHYTIIENICPKDESVKFYSPKRVHFPIPQADMDKKR FSFVFKPVFNTSLLFLQCELTLCTKMEKHPQKLPKCVPPDEACTS LDASIIWAMMQNKKTFTKPLAVIHHEAESKEKGPSMKEPNPISP PIFHGLDTLTVMGIAFAAFVIGALLTGALWYIYSHTGETAGRQQ VPTSPPASENSSAAHSIGSTQSTPCSSSSTA |
| SEQ ID NO: 3108 | Human TGFBR3 fragment 1 | GPEPGALCELSPVSASHPVQALMESFTVLSGCASRGTTGLPQEV HVLNLRTAGQGPGQLQREVTLHLNPISSVHIHHKSVVFLLNSPH PLVWHLKTERLATGVSRLFLVSEGSVVQFSSANFSLTAETEERN FPHGNEHLLNWARKEYGAVTSFTELKIARNIYIKVGEDQVFPPK CNIGKNFLSLNYLAEYLQPKAAEGCVMSSQPQNEEVHIIELITPN SNPYSAFQVDITIDIRPSQEDLEVVKNLILILKCKKSVNWVIKSFD VKGSLKIIAPNSIGFGKESERSMTMTKSIRDDIPSTQGNLVKWAL DNGYSPITSYTMAPVANRFHLRLENNAEEMGDEEVHTIPPELRIL LDPGALPALQNPPIRGGEGQNGGLPFPFPDISRRVWNEEGEDGL PRPKDPVIPSIQLFPGLREPEEVQGSVDIALSVKCDNEKMIVAVE KDSFQASGYSGMDVTLLDPTCKAKMNGTHFVLESPLNGCGTRP |
| | | RWSALDGVVYYNSIVIQVPALGDSSGWPDGYEDLESGDNGFPG DMDEGDASLFTRPEIVVFNCSLQQVRNPSSFQEQPHGNITFNME LYNTDLFLVPSQGVFSVPENGHVYVEVSVTKAEQELGFAIQTCF ISPYSNPDRMSHYTIIENICPKDESVKFYSPKRVHFPIPQADMDK KRFSFVFKPVFNTSLLFLQCELTLCTKMEKHPQKLPKCVPPDEA CTSLDASIIWAMMQNKKTFTKPLAVIHHEAESKEKGPSMKEPNP ISPPIFHGLDTLTV |
| SEQ ID NO: 3192 | hCH1-hFc_Hole-3×4GS-TGFbR2 | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKRVEPKSCDKTHTCPPCPAPELLGGPSVFLFPPKPKDTL MISRTPEVTCVVVDVSHEDPEVKFNWYVDGVEVHNAKTKPREE QYNSTYRVVSVLTVLHQDWLNGKEYKCKVSNKALPAPIEKTIS KAKGQPREPQVCTLPPSREEMTKNQVSLSCAVKGFYPSDIAVE WESNGQPENNYKTTPPVLDSDGSFFLVSKLTVDKSRWQQGNVF SCSVMHEALHNHYTQKSLSLSPGXGGGGSGGGGSGGGGSIPPH VQKSVNNDMIVTDNNGAVKFPQLCKFCDVRFSTCDNQKSCMS NCSITSICEKPQEVCVAVWRKNDENITLETVCHDPKLPYHDFILE DAASPKCIMKEKKKPGETFFMCSCSSDECNDNIIFSEEYNTSNPD, wherein X is K or absent |
| SEQ ID NO: 3193 | hCH1-hFc_Knob-3×4GS-TGFbR2 | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKRVEPKSCDKTHTCPPCPAPELLGGPSVFLFPPKPKDTL MISRTPEVTCVVVDVSHEDPEVKFNWYVDGVEVHNAKTKPREE QYNSTYRVVSVLTVLHQDWLNGKEYKCKVSNKALPAPIEKTIS KAKGQPREPQVYTLPPCREEMTKNQVSLWCLVKGFYPSDIAVE WESNGQPENNYKTTPPVLDSDGSFFLYSKLTVDKSRWQQGNVF SCSVMHEALHNHYTQKSLSLSPGXGGGGSGGGGSGGGGSIPPH VQKSVNNDMIVTDNNGAVKFPQLCKFCDVRFSTCDNQKSCMS NCSITSICEKPQEVCVAVWRKNDENITLETVCHDPKLPYHDFILE DAASPKCIMKEKKKPGETFFMCSCSSDECNDNIIFSEEYNTSNPD, wherein X is K or absent |
| SEQ ID NO: 3194 | hFc_Hole-3×4GS-TGFbR2 | DKTHTCPPCPAPELLGGPSVFLFPPKPKDTLMISRTPEVTCVVVD VSHEDPEVKFNWYVDGVEVHNAKTKPREEQYNSTYRVVSVLT VLHQDWLNGKEYKCKVSNKALPAPIEKTISKAKGQPREPQVCT LPPSREEMTKNQVSLSCAVKGFYPSDIAVEWESNGQPENNYKTT PPVLDSDGSFFLVSKLTVDKSRWQQGNVFSCSVMHEALHNHYT QKSLSLSPGXGGGGSGGGGSGGGGSIPPHVQKSVNNDMIVTDN NGAVKFPQLCKFCDVRFSTCDNQKSCMSNCSITSICEKPQEVCV AVWRKNDENITLETVCHDPKLPYHDFILEDAASPKCIMKEKKKP GETFFMCSCSSDECNDNIIFSEEYNTSNPD, wherein X is K or absent |
| SEQ ID NO: 3195 | hFc_Knob-3×4GS-TGFbR2 | DKTHTCPPCPAPELLGGPSVFLFPPKPKDTLMISRTPEVTCVVVD VSHEDPEVKFNWYVDGVEVHNAKTKPREEQYNSTYRVVSVLT VLHQDWLNGKEYKCKVSNKALPAPIEKTISKAKGQPREPQVYT LPPCREEMTKNQVSLWCLVKGFYPSDIAVEWESNGQPENNYKT TPPVLDSDGSFFLYSKLTVDKSRWQQGNVFSCSVMHEALHNHY TQKSLSLSPGXGGGGSGGGGSGGGGSIPPHVQKSVNNDMIVTD NNGAVKFPQLCKFCDVRFSTCDNQKSCMSNCSITSICEKPQEVC VAVWRKNDENITLETVCHDPKLPYHDFILEDAASPKCIMKEKK KPGETFFMCSCSSDECNDNIIFSEEYNTSNPD, wherein X is K or absent |
| SEQ ID NO: 3196 | TGFbR2-3×4GS-hCH1-hFc Hole | IPPHVQKSVNNDMIVTDNNGAVKFPQLCKFCDVRFSTCDNQKS CMSNCSITSICEKPQEVCVAVWRKNDENITLETVCHDPKLPYHD |
| | | FILEDAASPKCIMKEKKKPGETFFMCSCSSDECNDNIIFSEEYNTS NPDGGGGSGGGGSGGGGSASTKGPSVFPLAPSSKSTSGGTAALG CLVKDYFPEPVTVSWNSGALTSGVHTFPAVLQSSGLYSLSSVVT VPSSSLGTQTYICNVNHKPSNTKVDKRVEPKSCDKTHTCPPCPA PELLGGPSVFLFPPKPKDTLMISRTPEVTCVVVDVSHEDPEVKFN WYVDGVEVHNAKTKPREEQYNSTYRVVSVLTVLHQDWLNGK EYKCKVSNKALPAPIEKTISKAKGQPREPQVCTLPPSREEMTKN QVSLSCAVKGFYPSDIAVEWESNGQPENNYKTTPPVLDSDGSFF LVSKLTVDKSRWQQGNVFSCSVMHEALHNHYTQKSLSLSPGX, wherein X is K or absent |
| SEQ ID NO: 3197 | TGFbR2-3×4GS-hCH1-hFc_Knob | IPPHVQKSVNNDMIVTDNNGAVKFPQLCKFCDVRFSTCDNQKS CMSNCSITSICEKPQEVCVAVWRKNDENITLETVCHDPKLPYHD FILEDAASPKCIMKEKKKPGETFFMCSCSSDECNDNIIFSEEYNTS NPDGGGGSGGGGSGGGGSASTKGPSVFPLAPSSKSTSGGTAALG CLVKDYFPEPVTVSWNSGALTSGVHTFPAVLQSSGLYSLSSVVT VPSSSLGTQTYICNVNHKPSNTKVDKRVEPKSCDKTHTCPPCPA PELLGGPSVFLFPPKPKDTLMISRTPEVTCVVVDVSHEDPEVKFN WYVDGVEVHNAKTKPREEQYNSTYRVVSVLTVLHQDWLNGK EYKCKVSNKALPAPIEKTISKAKGQPREPQVYTLPPCREEMTKN QVSLWCLVKGFYPSDIAVEWESNGQPENNYKTTPPVLDSDGSFF LYSKLTVDKSRWQQGNVFSCSVMHEALHNHYTQKSLSLSPGX, wherein X is K or absent |
| SEQ ID NO: 3198 | TGFbR2-3×4GS-hCLIg_vl | IPPHVQKSVNNDMIVTDNNGAVKFPQLCKFCDVRFSTCDNQKS CMSNCSITSICEKPQEVCVAVWRKNDENITLETVCHDPKLPYHD FILEDAASPKCIMKEKKKPGETFFMCSCSSDECNDNIIFSEEYNTS NPDGGGGSGGGGSGGGGSGQPKANPTVTLFPPSSEELQANKAT LVCLISDFYPGAVTVAWKADGSPVKAGVETTKPSKQSNNKYAA SSYLSLTPEQWKSHRSYSCQVTHEGSTVEKTVAPTECS |
| SEQ ID NO: 3199 | TGFβR2-3×4GS-hCLIg_vk | IPPHVQKSVNNDMIVTDNNGAVKFPQLCKFCDVRFSTCDNQKS CMSNCSITSICEKPQEVCVAVWRKNDENITLETVCHDPKLPYHD FILEDAASPKCIMKEKKKPGETFFMCSCSSDECNDNIIFSEEYNTS NPDGGGGSGGGGSGGGGSRTVAAPSVFIFPPSDEQLKSGTASVV CLLNNFYPREAKVQWKVDNALQSGNSQESVTEQDSKDSTYSLS STLTLSKADYEKHKVYACEVTHQGLSSPVTKSFNRGEC |

### Stromal Modifying Moieties

In other embodiments, the anti-NKp30 antibody molecule (e.g., the multispecific antibody molecule) further comprises one or more stromal modifying moieties. Stromal modifying moieties described herein include moieties (e.g., proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

Exemplary amino acid and nucleotide sequences for stromal modifying moieties are disclosed in WO 2017/165464, see e.g., pages 131-136, 188-193, incorporated herein by reference.

### Nucleic Acids

Nucleic acids encoding the aforementioned antibody molecules, e.g., multispecific or multifunctional molecules. are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety disclosed herein.

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding a multispecific or multifunctional molecule described herein. In one embodiment, the vectors comprise nucleotides encoding a multispecific or multifunctional molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses

Methods described herein include treating a disorder, e.g., a cancer, an autoimmune or inflammatory disorder, or an infectious disorder, in a subject by using an anti-NKp30 antibody molecule, e.g., a multispecific molecule, described herein, e.g., using a pharmaceutical composition described herein. Also provided are methods for reducing or ameliorating a symptom of a disorder, e.g., a cancer, an autoimmune or inflammatory disorder, or an infectious disorder, in a subject, as well as methods for inhibiting the growth of a diseased cell, e.g., cancer cell, and/or killing or depleting one or more diseased cells, e.g., cancer cells. In embodiments, the methods described herein decrease the size of a tumor and/or decrease the number of cancer cells in a subject administered with a described herein or a pharmaceutical composition described herein.

In embodiments, the antibody molecule, e.g., multispecific molecules or pharmaceutical composition, is administered to the subject parenterally. In embodiments, the antibody molecule or pharmaceutical composition is administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Cancers

In embodiments, the cancer is a hematological cancer, a solid tumor or a metastatic lesion thereof. In some embodiments, the anti-NKp30 antibody molecule used to treat the cancer further comprises a tumor targeting moiety, e.g., a tumor targeting moiety as described herein.

In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi’s sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In certain embodiments, the cancer is an epithelial, mesenchymal or hematologic malignancy. In certain embodiments, the cancer treated is a solid tumor (e.g., carcinoid, carcinoma or sarcoma), a soft tissue tumor (e.g., a heme malignancy), and a metastatic lesion, e.g., a metastatic lesion of any of the cancers disclosed herein. In one embodiment, the cancer treated is a fibrotic or desmoplastic solid tumor, e.g., a tumor having one or more of: limited tumor perfusion, compressed blood vessels, fibrotic tumor interstitium, or increased interstitial fluid pressure. In one embodiment, the solid tumor is chosen from one or more of pancreatic (e.g., pancreatic adenocarcinoma or pancreatic ductal adenocarcinoma), breast, colon, colorectal, lung (e.g., small cell lung cancer (SCLC) or non-small cell lung cancer (NSCLC)), skin, ovarian, liver cancer, esophageal cancer, endometrial cancer, gastric cancer, head and neck cancer, kidney, or prostate cancer.

Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject’s body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin’s Disease, Adult Hodgkin’s Lymphoma, Adult Lymphocytic Leukemia, Adult Non-Hodgkin’s Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin’s Disease, Childhood Hodgkin’s Lymphoma, Childhood Hypothalamic and Visual Pathway Glioma, Childhood Lymphoblastic Leukemia, Childhood Medulloblastoma, Childhood Non-Hodgkin’s Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing’s Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher’s Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin’s Disease, Hodgkin’s Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi’s Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin’s Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom’s Macroglobulinemia, Wilms’ Tumor, and any other hyperproliferative disease, besides neoplasia, located in an organ system listed above.

In other embodiments, the multispecific molecule, as described above and herein, is used to treat a hyperproliferative disorder, e.g., a hyperproliferative connective tissue disorder (e.g., a hyperproliferative fibrotic disease). In one embodiment, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the eye, lung, liver, heart, kidney, pancreas, skin and other organs). In other embodiments, the disorder is chosen from liver cirrhosis or tuberculosis. In other embodiments, the disorder is leprosy.

In embodiments, the multispecific molecules (or pharmaceutical composition) are administered in a manner appropriate to the disease to be treated or prevented. The quantity and frequency of administration will be determined by such factors as the condition of the patient, and the type and severity of the patient’s disease. Appropriate dosages may be determined by clinical trials. For example, when “an effective amount” or “a therapeutic amount” is indicated, the precise amount of the pharmaceutical composition (or multispecific molecules) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the cancer is a myeloproliferative neoplasm, e.g., primary or idiopathic myelofibrosis (MF), essential thrombocytosis (ET), polycythemia vera (PV), or chronic myelogenous leukemia (CML). In embodiments, the cancer is myelofibrosis. In embodiments, the subject has myelofibrosis. In embodiments, the subject has a calreticulin mutation, e.g., a calreticulin mutation disclosed herein. In embodiments, the subject does not have the JAK2-V617F mutation. In embodiments, the subject has the JAK2-V617F mutation. In embodiments, the subject has a MPL mutation. In embodiments, the subject does not have a MPL mutation.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi’s sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

### Inflammatory and Autoimmune Disorders

In some embodiments, the anti-NKp30 antibody molecules, e.g., the multispecific antibody molecules, disclosed herein can be used to treat inflammatory and autoimmune diseases, and graft vs. host disease (GvHD). In some embodiments, the antibody molecules, e.g., the multispecific antibody molecules, disclosed herein deplete autoreactive T cells, e.g., by directing an NK cell, e.g., an NKp30-expressing cell, to an autoreactive T cell. In some embodiments, the anti-NKp30 antibody molecule further comprises a binding specificity that binds to an autoreactive T cell, e.g., an antigen present on the surface of an autoreactive T cell that is associated with the inflammatory or autoimmune disorder.

As used herein, the term “autoimmune” disease, disorder, or condition refers to a disease where the body’s immune system attacks its own cells or tissues. An autoimmune disease can result in the production of autoantibodies that are inappropriately produced and/or excessively produced to a self-antigen or autoantigen. Autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases. In some embodiments, the autoimmune disease is mediated by T cells, B cells, innate immune cells (e.g., macrophages, eosinophils, or natural killer cells), or complement-mediated pathways.

Examples of autoimmune disorders that may be treated by administering the antibodies of the present invention include, but are not limited to, alopecia greata, ankylosing spondylitis, antiphospholipid syndrome, autoimmune Addison’s disease, autoimmune diseases of the adrenal gland, autoimmune hemolytic anemia, autoimmune hepatitis, autoimmune oophoritis and orchitis, autoimmune thrombocytopenia, Behcet’s disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn’s disease, discoid lupus, essential mixed cryoglobulinemia, fibromyalgia-fibromyositis, glomerulonephritis, Graves’ disease, Guillain-Barre, Hashimoto’s thyroiditis, idiopathic pulmonary fibrosis, idiopathic thrombocytopenia purpura (ITP), IgA neuropathy, juvenile arthritis, lichen planus, lupus erthematosus, Meniere’s disease, mixed connective tissue disease, multiple sclerosis, Neuromyelitis optica (NMO), type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld’s phenomenon, Reiter’s syndrome, Rheumatoid arthritis, sarcoidosis, scleroderma, Sjogren’s syndrome, stiff-man syndrome, systemic lupus erythematosus, lupus erythematosus, takayasu arteritis, temporal arteristis/giant cell arteritis, transverse myelitis, ulcerative colitis, uveitis, vasculitides such as dermatitis herpetiformis vasculitis, vitiligo, and Wegener’s granulomatosis. In some embodiments, the autoimmune disorder is SLE or Type-1 diabetes.

Examples of inflammatory disorders which can be prevented, treated or managed in accordance with the methods of the invention include, but are not limited to, asthma, encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections.

Thus, the anti- NKp30 antibody molecules, e.g., multispecific molecules, of the present invention have utility in the treatment of inflammatory and autoimmune diseases.

### Infectious Diseases

In some embodiments, the anti-NKp30 antibody molecules, e.g., the multispecific antibody molecules, disclosed herein can be used to treat infectious diseases. In some embodiments, the antibody molecules, e.g., the multispecific antibody molecules, disclosed herein deplete cells expressing a viral or bacterial antigen. In some embodiments, the anti-NKp30 antibody molecule further comprises a binding specificity that binds to an antigen present on the surface of an infected cell, e.g., a viral infected cell.

Some examples of pathogenic viruses causing infections treatable by methods include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus. In one embodiment, the infection is an influenza infection.

In another embodiment, the infection is a hepatitis infection, e.g., a Hepatitis B or C infection.

Exemplary viral disorders that can be treated include, but are not limited to, Epstein Bar Virus (EBV), influenza virus, HIV, SIV, tuberculosis, malaria and HCMV.

Some examples of pathogenic bacteria causing infections treatable by methods of the invention include syphilis, chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria. The anti-NKp30 antibody molecules can be used in combination with existing treatment modalities for the aforesaid infections. For example, Treatments for syphilis include penicillin (e.g., penicillin G.), tetracycline, doxycycline, ceftriaxone and azithromycin.

### Diagnostic Uses

In one aspect, the present invention provides a diagnostic method for detecting the presence of a NKp30 protein in vitro (e.g., in a biological sample, such as a tissue biopsy, e.g., from a cancerous tissue) or in vivo (e.g., in vivo imaging in a subject). The method includes: (i) contacting the sample with an antibody molecule described herein, or administering to the subject, the antibody molecule; (optionally) (ii) contacting a reference sample, e.g., a control sample (e.g., a control biological sample, such as plasma, tissue, biopsy) or a control subject)); and (iii) detecting formation of a complex between the antibody molecule, and the sample or subject, or the control sample or subject, wherein a change, e.g., a statistically significant change, in the formation of the complex in the sample or subject relative to the control sample or subject is indicative of the presence of NKp30 in the sample. The antibody molecule can be directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

The term “sample,” as it refers to samples used for detecting polypeptides includes, but is not limited to, cells, cell lysates, proteins or membrane extracts of cells, body fluids, or tissue samples.

Complex formation between the antibody molecule and NKp30 can be detected by measuring or visualizing either the binding molecule bound to the NKp30 antigen or unbound binding molecule. Conventional detection assays can be used, e.g., an enzyme-linked immunosorbent assays (ELISA), a radioimmunoassay (RIA) or tissue immunohistochemistry. Alternative to labeling the antibody molecule, the presence of NKp30 can be assayed in a sample by a competition immunoassay utilizing standards labeled with a detectable substance and an unlabeled antibody molecule. In this assay, the biological sample, the labeled standards and the antibody molecule are combined and the amount of labeled standard bound to the unlabeled binding molecule is determined. The amount of NKp30 in the sample is inversely proportional to the amount of labeled standard bound to the antibody molecule.

### EXAMPLES

The Examples below are set forth to aid in the understanding of the inventions but are not intended to, and should not be construed to, limit its scope in any way.

### Example 1: Immunization of Armenian Hamster to Generate Anti-NKp30 Antibodies

Briefly, armenian hamsters were immunized with the extracellular domain of human NKp30 protein in complete Freund’s adjuvant and boosted twice on day 14 and day 28 with NKp30 in incomplete Freund’s adjuvant (IFA). On day 56 one more boost in IFA was given and the animals harvested three days later. Spleens were collected and fused with P3X63Ag8.653 murine myeloma cell line. 0.9 × 10^5 cells/well in 125 ul were seated in 96 well plate and fed with 125 µl of I-20 + 2ME + HAT (IMDM (4 g/L glucose) supplemented with 20% fetal bovine serum, 4 mM L-glutamine, 1 mM sodium pyruvate, 50 U penicillin, 50 µg streptomycin and 50 µM 2-ME in the absence or presence of HAT or HT for selection, and Hybridoma Cloning Factor (1% final) on days 7, 11 and thereafter as needed. At approximately 2 weeks after fusion (cells are about 50% confluent), supernatant was collected and assayed for binding.

### Example 2: Hybridoma Screen for NKp30 mAbs

Expi293 cells were transfected with BG160 (hNKp30 cell antigen) 18 hours prior to screening. The day of screening, transfected cells were diluted to 0.05 ×10^{^6}/mL and anti-Armenian hamster Fc Alexa Fluor 488 added to a final concentration of 0.4 ug/mL. 50 uL (2,500 cells) of this mixture was added to each well of a 384 well plate. The same density of untransfected 293 cells with secondary were used as a negative control. 5 uL of hybridoma supematant was added to the cell mixture and the plate incubated for 1 hour at 37° C. The plates were then imaged on Mirrorball. Positive clones were identified and subcloned by serial dilution to obtain clonal selected hybridoma. After reconfirmation using the same protocols the hybridoma cells were harvested and the corresponding heavy and light chain sequences recovered. The DNA was subcloned into pcDNA3.4 for subsequent expression of the corresponding antibodies and further validation.

### Example 3: Binding of NKp30 Antibodies to NK92 Cells

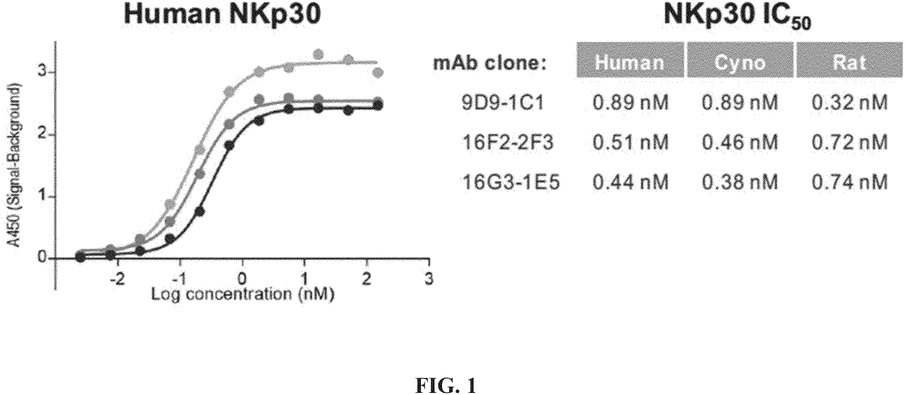

NK-92 cells were washed with PBS containing 0.5% BSA and 0.1% sodium azide (staining buffer) and added to 96-well V-bottom plates with 200,000 cells/well. Hamster NKp30 antibodies were added to the cells in 2.0 fold serial dilutions and incubated for 1 hour at room temperature. The plates were washed twice with staining buffer. The secondary antibody against hamster Fc conjugated to AF647 (Jackson, 127-605-160) was added at 1:100 dilution (1.4 mg/ml stock) and incubated with the cells for 30 minutes at 4° C. followed by washing with staining buffer. Cells were subsequently were fixed for 10 minutes with 4% paraformaldehyde at room temperature. The plates were read on CytoFLEX LS (Beckman Coulter). Data was calculated as the percent-AF747 positive population (FIG. 1).

### Example 4: Bioassay to Measure Activity of NKp30 Antibodies Using NK92 Cell Line

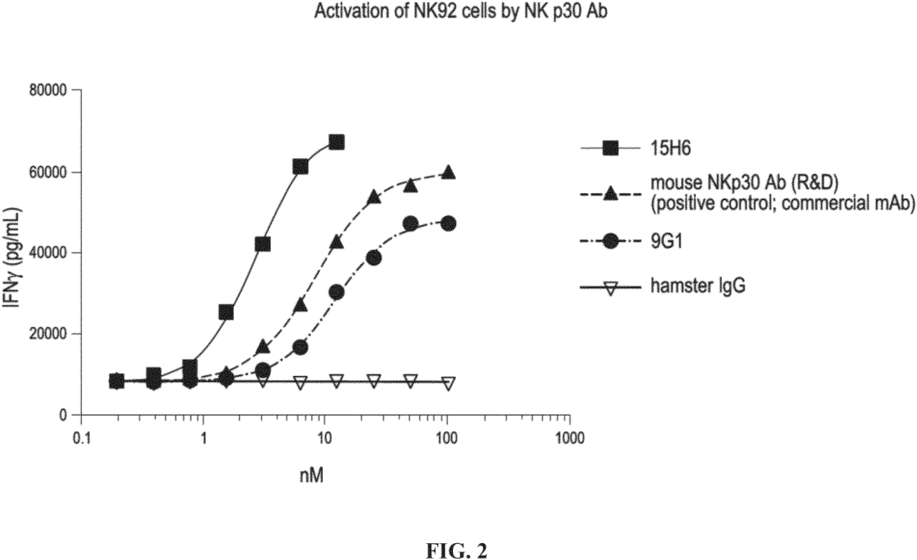

NKp30 antibodies were three-fold serially diluted in PBS and incubated at 2-8° C. overnight in flat bottom 96 well plates. Plates were washed twice in PBS and 40,000 NK-92 cells were added in growth medium containing IL-2. Plates were incubated at 37° C., 5% CO2, humidified incubator for 16-24 hours before supernatants were collected. IFNγ levels in supernatants was measured following MSD assay instructions (FIG. 2). Supernatant collected from cells incubated with hamster isotype IgG was used as negative control and supernatants from cells incubated with NKp30 monoclonal antibody (R&D, clone 210847) was utilized as a positive control. Data were generated using hamster anti-NKp30 mABs.

### Example 5: Generation and Characterization of Humanized Anti-NKp30 Antibodies

A series of hamster anti-NKp30 antibodies were selected. These antibodies were shown to bind to human NKp30 and cynomolgus NKp30 and induce IFNγ production from NK-90 cells (data not shown). The VH and VL sequences of exemplary hamster anti-NKp30 antibodies 15E1, 9G1, 15H6, 9D9, 3A12, and 12D10 are disclosed in Table 9. The VH and VL sequences of exemplary humanized anti-NKp30 antibodies based on 15E1, 9G1, and 15H6 are also disclosed in Table 9. The Kabat CDRs of these antibodies are disclosed in Table 18 and Table 8.

Two humanized constructs based on 15E1 were selected. The first construct BJM0407 is a Fab comprising a heavy chain variable region comprising the amino acid sequence of SEQ ID NO: 7302 and a lambda light chain variable region comprising the amino acid sequence of SEQ ID NO: 7305. Its corresponding scFv construct BJM0859 comprises the amino acid sequence of SEQ ID NO: 7310. The second construct BJM0411 is a Fab comprising a heavy chain variable region comprising the amino acid sequence of SEQ ID NO: 7302 and a kappa light chain variable region comprising the amino acid sequence of SEQ ID NO: 7309. Its corresponding scFv construct BJM0860 comprises the amino acid sequence of SEQ ID NO: 7311. BJM0407 and BJM0411 showed comparable biophysical characteristics, e.g., binding affinity to NKp30 and thermal stability. The scFv constructs BJM0859 and BJM0860 also showed comparable biophysical properties.

### Example 6: Biacore Analysis of Exemplary Anti-NKp30 Antibody Molecules

In this example, a series of exemplary anti-NKp30 antibody molecules were analyzed for their binding affinity for NKp30. Briefly, surface plasmon resonance (SPR) measurements were performed by using the BIAcore T200. Human NKp30 (BKM0179) was immobilized on a CM5 chip via anti-mouse Fc antibody to a response of 50 RU. Each exemplary antibody construct were injected at concentrations of 3.9, 7.8, 15.6, 31.2, 62.5, and 125 nM, and at a flow rate of 20 µl/min, over the surface on which the human NKp30 was immobilized. The data was fit using a 1:1 binding model.

As shown in Table 19, most of the exemplary antibodies showed preserved affinity to human NKp30 compared to the parental antibody.

TABLE 19

Biacore results

| | | |
|---|---|---|
| Construct | Description | Human Nkp30 (BKM0179) |
| BJM1078 | BJM0407 Parental | 1.48 nM |
| BJM1079 | BJM0411 Parental | 1.26 nM |
| BKM0138 | BJM0411 VL-N95A | 3.2 nM |
| BKM0139 | BJM0411 VL-D92A | 3.2 nM |
| BKM0140 | BJM0407 VL-D92A | 3.3 nM |
| BKM0141 | BJM0407 VL-N95A | 3.0 nM |
| BKM0142 | BJM0411 VH-N60A | 1.28 nM |
| BKM0143 | BJM0407 VH-N60A | 1.45 nM |
| BKM0144 | BJM0411 VH-N60A-VL-D92A-N95A | 6.4 nM |
| BKM0145 | BJM0407 VH-N60A-VL-D92A-N95A | 4.2 nM |

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### EXEMPLARY EMBODIMENTS

Additional features of any of the aforesaid multifunctional molecules, nucleic acids, vectors, host cells, or methods include one or more of the following exemplary embodiments.

Those skilled in the art will recognize, or be able to ascertain using no more than routine experimentation, many equivalents to the specific embodiments of the invention described herein. Such equivalents are intended to be encompassed by the following exemplary embodiments.

### Exemplary Embodiment 1

Disclosed herein are antibody molecules (e.g., humanized antibody molecules) that bind to NKp30 with high affinity and specificity. Nucleic acid molecules encoding the antibody molecules, expression vectors, host cells and methods for making the antibody molecules are also provided. Multi- or bispecific or multifunctional antibody molecules and pharmaceutical compositions comprising the antibody molecules are also provided. The anti- NKp30antibody molecules disclosed herein can be used (alone or in combination with other agents or therapeutic modalities) to treat, prevent and/or diagnose disorders, such as cancerous disorders (e.g., solid and soft-tissue tumors), as well as autoimmune and infectious diseases. Thus, compositions and methods for detecting NKp30, as well as methods for treating various disorders including cancer, autoimmune and/or infectious diseases, using the anti- NKp30 antibody molecules are disclosed herein.

Accordingly, in one aspect, the invention features an antibody molecule (e.g., an isolated or recombinant antibody molecule), comprising one or more sequences according to the following enumerated embodiments. Additional features of any of the disclosed antibody molecules, multifunctional molecules, nucleic acids, vectors, host cells, or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

In an aspect, the disclosure features an isolated antibody molecule that binds to NKp30, comprising:
(a) a heavy chain complementarity determining region 1 (VHCDR1), a heavy chain complementarity determining region 2 (VHCDR2), and/or a heavy chain complementarity determining region 3 (VHCDR3) described in Table 8A, or a sequence with no more than 1, 2, 3, or 4 mutations, e.g., substitutions, additions, or deletions; and/or
(b) a light chain complementarity determining region 1 (VLCDR1), a light chain complementarity determining region 2 (VLCDR2), and/or a light chain complementarity determining region 3 (VLCDR3) described in Table 8B, or a sequence with no more than 1, 2, 3, or 4 mutations, e.g., substitutions, additions, or deletions.

In some embodiments, the VHCDR1 comprises the amino acid sequence of any of SEQ ID NOs: 19, 33, 47, 61, 7397, 89, 103, or 116, or an amino acid sequence with no more than 1, 2, 3, or 4 mutations, e.g., substitutions, additions, or deletions.

In some embodiments, the VHCDR2 comprises the amino acid sequence of any of SEQ ID NOs: 21, 35, 49, 63, 7399, 91, 105, or 118, or an amino acid sequence with no more than 1, 2, 3, or 4 mutations, e.g., substitutions, additions, or deletions.

In some embodiments, the VHCDR3 comprises the amino acid sequence of any of SEQ ID NOs: 23, 37, 51, 65, 79, 93, 107, or 120, or an amino acid sequence with no more than 1, 2, 3, or 4 mutations, e.g., substitutions, additions, or deletions.

In some embodiments, the VLCDR1 comprises the amino acid sequence of any of SEQ ID NOs: 26, 40, 54, 68, 82, 96, 110, or 123, or an amino acid sequence with no more than 1, 2, 3, or 4 mutations, e.g., substitutions, additions, or deletions.

In some embodiments, the VLCDR2 comprises the amino acid sequence of any of SEQ ID NOs: 28, 7393, 56, 70, 84, 98, 112, or 125, or an amino acid sequence with no more than 1, 2, 3, or 4 mutations, e.g., substitutions, additions, or deletions.

In some embodiments, the VLCDR3 comprises the amino acid sequence of any of SEQ ID NOs: 30, 7395, 58, 72, 86, 100, 113, or 127, or an amino acid sequence with no more than 1, 2, 3, or 4 mutations, e.g., substitutions, additions, or deletions.

In some embodiments, the antibody molecule comprises:
(a) a heavy chain framework region 1 (VHFWR1), a heavy chain framework region 2 (VHFWR1), a heavy chain framework region 3 (VHFWR3), and/or a heavy chain framework region 4 (VHFWR4) described in Table 8A, or an amino acid sequence with no more than 1, 2, 3, or 4 mutations, e.g., substitutions, additions, or deletions; and/or
(b) a light chain framework region 1 (VLFWR1), a light chain framework region 2 (VLFWR1), a light chain framework region 3 (VLFWR3), and/or a light chain framework region 4 (VLFWR4) described in Table 8B, or an amino acid sequence with no more than 1, 2, 3, or 4 mutations, e.g., substitutions, additions, or deletions.

In some embodiments, the antibody molecule comprises:
(a) a heavy chain variable region (VH) described in Table 9, e.g., a VH comprising the amino acid sequence of any of SEQ ID NOs: 1-8, or an amino acid sequence having at least about 75%, 80%, 85%, 90%, 95%, or 99% sequence identity thereto; and/or
(b) a light chain variable region (VL) described in Table 9, e.g., a VL comprising the amino acid sequence of any of SEQ ID NOs: 9-16, or an amino acid sequence having at least about 75%, 80%, 85%, 90%, 95%, or 99% sequence identity thereto.

In some embodiments, the antibody molecule comprises an amino acid sequence described in Table 10, e.g., the amino acid sequence of any of SEQ ID NOs: 17, and 7386-7392, or an amino acid sequence having at least about 75%, 80%, 85%, 90%, 95%, or 99% sequence identity thereto.

In an aspect, the disclosure features a multispecific molecule comprising an antibody molecule described herein.

In some embodiments, the multispecific molecule further comprises one, two, three, four or more of: (a) a tumor targeting moiety, e.g., as described herein; (b) a cytokine molecule, e.g., as described herein; (c) a T cell engager, e.g., as described herein; or (d) a stromal modifying moiety, e.g., as described herein.

In some embodiments, the multispecific molecule further comprises a binding specificity that binds to an autoreactive T cell, e.g., an antigen present on the surface of an autoreactive T cell that is associated with the inflammatory or autoimmune disorder.

In some embodiments, the multispecific molecule further comprises a binding specificity that binds to an infected cell, e.g., a viral or bacterial infected cell.

In some embodiments, the antibody molecule is a monospecific antibody molecule, a bispecific antibody molecule, or a trispecific antibody molecule. In some embodiments, the multispecific molecule is a bispecific antibody molecule, or a trispecific antibody molecule.

In some embodiments, the antibody molecule or the multispecific molecule is a monovalent antibody molecule, a bivalent antibody molecule, or a trivalent antibody molecule.

In some embodiments, the antibody molecule or the multispecific molecule is a full antibody (e.g., an antibody that includes at least one, and preferably two, complete heavy chains, and at least one, and preferably two, complete light chains), or an antigen-binding fragment (e.g., a Fab, F(ab′)₂, Fv, a single chain Fv, a single domain antibody, a diabody (dAb), a bivalent antibody, or bispecific antibody or fragment thereof, a single domain variant thereof, or a camelid antibody).

In some embodiments, the antibody molecule or the multispecific molecule comprises a heavy chain constant region chosen from IgG1, IgG2, IgG3, or IgG4, or a fragment thereof.

In some embodiments, the antibody molecule or the multispecific molecule comprises a light chain constant region chosen from the light chain constant regions of kappa or lambda, or a fragment thereof.

In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function.

In some embodiments, an interface of a first and second immunoglobulin chain constant regions (e.g., Fc region) is altered, e.g., mutated, to increase or decrease dimerization, e.g., relative to a non-engineered interface.

In some embodiments, the dimerization of the immunoglobulin chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface.

In some embodiments, the immunoglobulin chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1.

In some embodiments, the immunoglobulin chain constant region (e.g., Fc region) comprises an amino acid substitution chosen from: T366S, L368A, or Y407V (e.g., corresponding to a cavity or hole), or T366W (e.g., corresponding to a protuberance or knob), or a combination thereof.

In some embodiments, the antibody molecule or the multispecific molecule further comprises a linker, e.g., a linker between one or more of: the targeting moiety and the cytokine molecule or the stromal modifying moiety, the targeting moiety and the immune cell engager, the cytokine molecule or the stromal modifying moiety, and the immune cell engager, the cytokine molecule or the stromal modifying moiety and the immunoglobulin chain constant region (e.g., the Fc region), the targeting moiety and the immunoglobulin chain constant region, or the immune cell engager and the immunoglobulin chain constant region.

In some embodiments, the linker is selected from: a cleavable linker, a non-cleavable linker, a peptide linker, a flexible linker, a rigid linker, a helical linker, or a non-helical linker. In some embodiments, the linker is a peptide linker. In some embodiments, the peptide linker comprises Gly and Ser.

In another aspect, the disclosure features an isolated nucleic acid molecule, which comprises the nucleotide sequence encoding any of the antibody molecules or multispecific or multifunctional molecules described herein, or a nucleotide sequence substantially homologous thereto (e.g., at least 95% to 99.9% identical thereto).

In another aspect, the disclosure features an isolated nucleic acid encoding an antibody molecule described herein or a multispecific molecule described herein.

In another aspect, the disclosure features a vector, e.g., an expression vector, comprising one or more of the nucleic acid molecules described herein.

In another aspect, the disclosure features a host cell comprising a nucleic acid molecule described herein or a vector described herein.

In another aspect, the disclosure features a method of making, e.g., producing, an antibody molecule described herein or a multispecific or multifunctional molecule polypeptide described herein, comprising culturing a host cell described herein, under suitable conditions, e.g., conditions suitable for gene expression and/or homo- or heterodimerization.

In another aspect, the disclosure features a pharmaceutical composition comprising an antibody molecule described herein or a multispecific or multifunctional molecule polypeptide described herein and a pharmaceutically acceptable carrier, excipient, or stabilizer.

In another aspect, the disclosure features a method of treating a cancer, comprising administering to a subject in need thereof an antibody molecule described herein or a multispecific or multifunctional molecule polypeptide described herein, wherein the antibody molecule or the multispecific or multifunctional molecule polypeptide is administered in an amount effective to treat the cancer.

In another aspect, the disclosure features an antibody molecule described herein or a multispecific or multifunctional molecule polypeptide described herein for use in treating cancer.

In some embodiments, the cancer is a solid tumor cancer, or a metastatic lesion. In some embodiments, the solid tumor cancer is one or more of pancreatic (e.g., pancreatic adenocarcinoma), breast, colorectal, lung (e.g., small or non-small cell lung cancer), skin, ovarian, or liver cancer. In some embodiments, the cancer is a hematological cancer.

In some embodiments, the method or use further comprising administering a second therapeutic treatment. In some embodiments, the second therapeutic treatment comprises a therapeutic agent (e.g., a chemotherapeutic agent, a biologic agent, hormonal therapy), radiation, or surgery. In some embodiments, the therapeutic agent is selected from: a chemotherapeutic agent, or a biologic agent.

In an aspect, the disclosure features a method of treating an autoimmune or an inflammatory disorder, comprising administering to a subject in need thereof an antibody molecule described herein or a multispecific or multifunctional molecule polypeptide described herein, wherein the antibody molecule or the multispecific or multifunctional molecule polypeptide is administered in an amount effective to treat the autoimmune or the inflammatory disorder.

In an aspect, the disclosure features an antibody molecule described herein or a multispecific or multifunctional molecule polypeptide described herein for use in treating an autoimmune or an autoinflammatory disorder.

In an aspect, the disclosure features a method of treating an infectious disorder, comprising administering to a subject in need thereof an antibody molecule described herein or a multispecific or multifunctional molecule polypeptide described herein, wherein the antibody molecule or the multispecific or multifunctional molecule polypeptide is administered in an amount effective to treat the infectious disorder.

In an aspect, the disclosure features an antibody molecule described herein or a multispecific or multifunctional molecule polypeptide described herein for use in treating an infectious disorder.

### Exemplary Embodiment 2

The present disclosure also includes any of the following enumerated embodiments:

1. An isolated antibody molecule that binds to NKp30, comprising:
(i) a heavy chain variable region (VH) comprising a heavy chain complementarity determining region 1 (VHCDR1) amino acid sequence of SEQ ID NO: 7313 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions, and/or a VHCDR3 amino acid sequence of SEQ ID NO: 7315 (or a sequence with no more than 1, 2, 3, or 4 mutations, e.g., substitutions, additions, or deletions; and/or
(ii) a light chain variable region (VL) comprising a light chain complementarity determining region 1 (VLCDR1) amino acid sequence of SEQ ID NO: 7326 (or a sequence with no more than 1, 2, 3, or 4 mutations, e.g., substitutions, additions, or deletions), a VLCDR2 amino acid sequence of SEQ ID NO: 7327 (or a sequence with no more than 1, 2, 3, or 4 mutations, e.g., substitutions, additions, or deletions), and/or a VLCDR3 amino acid sequence of SEQ ID NO: 7329 (or a sequence with no more than 1, 2, 3, or 4 mutations, e.g., substitutions, additions, or deletions).

2. The antibody molecule of embodiment 1, wherein the antigen binding domain comprises:
(i) a VH comprising the amino acid sequence of any of SEQ ID NOs: 7298 or 7300-7304 (or an amino acid sequence having at least about 75%, 80%, 85%, 90%, 95%, or 99% sequence identity to any of SEQ ID NOs: 7298 or 7300-7304), and/or
(ii) a VL comprising the amino acid sequence of any of SEQ ID NOs: 7299 or 7305-7309 (or an amino acid sequence having at least about 93%, 95%, or 99% sequence identity to any of SEQ ID NOs: 7299 or 7305-7309).

3. The antibody molecule of embodiment 2, wherein the antigen binding domain comprises:
(i)) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7305 (or an amino acid sequence having at least about 75%, 80%, 85%, 90%, 95%, or 99% sequence identity to 7305); or
(ii) a VH comprising the amino acid sequence of SEQ ID NO: 7302 (or an amino acid sequence having at least about 75%, 80%, 85%, 90%, 95%, or 99% sequence identity to 7302), and a VL comprising the amino acid sequence of SEQ ID NO: 7309 (or an amino acid sequence having at least about 75%, 80%, 85%, 90%, 95%, or 99% sequence identity to 7309).

4. The antibody molecule of any of embodiments 1-3, wherein the antigen binding domain comprises:
(i) an amino acid sequence of SEQ ID NO: 7310 (or an amino acid sequence having at least about 75%, 80%, 85%, 90%, 95%, or 99% sequence identity to 7310); or
(ii) an amino acid sequence of SEQ ID NO: 7311 (or an amino acid sequence having at least about 75%, 80%, 85%, 90%, 95%, or 99% sequence identity to 7311).

5. An isolated antibody molecule that binds to NKp30, comprising:
(i) a heavy chain variable region (VH) comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions), and
(ii) a light chain variable region (VL) comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions).

6. The antibody molecule of embodiment 5, wherein the antigen binding domain comprises: (i) a heavy chain variable region (VH) comprising a heavy chain complementarity determining region 1 (VHCDR1) amino acid sequence of SEQ ID NO: 6000, a VHCDR2 amino acid sequence of SEQ ID NO: 6001, and/or a VHCDR3 amino acid sequence of SEQ ID NO: 6002, and

(ii) a light chain variable region (VL) comprising a light chain complementarity determining region 1 (VLCDR1) amino acid sequence of SEQ ID NO: 6063, a VLCDR2 amino acid sequence of SEQ ID NO: 6064, and/or a VLCDR3 amino acid sequence of SEQ ID NO: 7293.

7. The antibody molecule of embodiment 5 or 6, wherein the antigen binding domain comprises:
(1) a heavy chain variable region (VH) comprising a heavy chain framework region 1 (VHFWR1) amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, or 6 mutations, e.g., substitutions, additions, or deletions, therefrom), or a VHFWR4 amino acid sequence of SEQ ID NO: 6006 (or a sequence with no more than 1, 2, 3, 4, 5, or 6 mutations, e.g., substitutions, additions, or deletions, therefrom), and/or
(2) a light chain variable region (VL) comprising a light chain framework region 1 (VLFWR1) amino acid sequence of SEQ ID NO: 6066 (or a sequence with no more than 1, 2, 3, 4, 5, or 6 mutations, e.g., substitutions, additions, or deletions, therefrom), a VLFWR2 amino acid sequence of SEQ ID NO: 6067 (or a sequence with no more than 1, 2, 3, 4, 5, or 6 mutations, e.g., substitutions, additions, or deletions, therefrom), a VLFWR3 amino acid sequence of SEQ ID NO: 7292 (or a sequence with no more than 1, 2, 3, 4, 5, or 6 mutations, e.g., substitutions, additions, or deletions, therefrom), or a VLFWR4 amino acid sequence of SEQ ID NO: 6069 (or a sequence with no more than 1, 2, 3, 4, 5, or 6 mutations, e.g., substitutions, additions, or deletions, therefrom).

8. The antibody molecule of embodiment 7, wherein the antigen binding domain comprises:

a heavy chain variable region (VH) comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, or a VHFWR4 amino acid sequence of SEQ ID NO: 6006, and (3) a light chain variable region (VL) comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, or a VLFWR4 amino acid sequence of SEQ ID NO: 6069.

9. The antibody molecule of any one of embodiments 5-8, wherein the antigen binding domain comprises:
(i) a VH comprising the amino acid sequence of SEQ ID NO: 6121 (or an amino acid sequence having at least about 75%, 80%, 85%, 90%, 95%, or 99% sequence identity to SEQ ID NO: 6121), and/or
(ii) a VL comprising the amino acid sequence of SEQ ID NO: 7294 (or an amino acid sequence having at least about 93%, 95%, or 99% sequence identity to SEQ ID NO: 7294).

10. The antibody molecule of any one of embodiments 5-9, wherein the antigen binding domain comprises a heavy chain comprising the amino acid sequence of SEQ ID NOs: 6148 or 6149 (or an amino acid sequence having at least about 75%, 80%, 85%, 90%, 95%, or 99% sequence identity to SEQ ID NOs: 6148 or 6149).

11. The antibody molecule of either of embodiments 5-10, wherein the antigen binding domain comprises a light chain comprising the amino acid sequence of SEQ ID NO: 6150 (or an amino acid sequence having at least about 75%, 80%, 85%, 90%, 95%, or 99% sequence identity to SEQ ID NO: 6150).

12. The antibody molecule of either of embodiments 5-11, wherein the antigen binding domain comprises a heavy chain comprising the amino acid sequence of SEQ ID NOs: 6148 or 6149 (or an amino acid sequence having at least about 75%, 80%, 85%, 90%, 95%, or 99% sequence identity to SEQ ID NOs: 6148 or 6149), and a light chain comprising the amino acid sequence of SEQ ID NO: 6150 (or an amino acid sequence having at least about 75%, 80%, 85%, 90%, 95%, or 99% sequence identity to SEQ ID NO: 6150).

13. The antibody molecule of any of embodiments 5-12, wherein the antigen binding domain comprises a heavy chain variable region (VH) comprising a heavy chain framework region 1 (VHFWR1) amino acid sequence of SEQ ID NO: 6014 (or a sequence with no more than 1, 2, 3, 4, 5, or 6 mutations, e.g., substitutions, additions, or deletions, therefrom), a VHFWR2 amino acid sequence of SEQ ID NO: 6015 (or a sequence with no more than 1, 2, 3, 4, 5, or 6 mutations, e.g., substitutions, additions, or deletions, therefrom), a VHFWR3 amino acid sequence of SEQ ID NO: 6016 (or a sequence with no more than 1, 2, 3, 4, 5, or 6 mutations, e.g., substitutions, additions, or deletions, therefrom), or a VHFWR4 amino acid sequence of SEQ ID NO: 6017 (or a sequence with no more than 1, 2, 3, 4, 5, or 6 mutations, e.g., substitutions, additions, or deletions, therefrom).

14. The antibody molecule of embodiment 13, wherein the antigen binding domain comprises a heavy chain variable region (VH) comprising a heavy chain framework region 1 (VHFWR1) amino acid sequence of SEQ ID NO: 6014, a VHFWR2 amino acid sequence of SEQ ID NO: 6015, a VHFWR3 amino acid sequence of SEQ ID NO: 6016, or a VHFWR4 amino acid sequence of SEQ ID NO: 6017.

15. The antibody molecule of embodiment 14, wherein the antigen binding domain comprises a VH comprising the amino acid sequence of SEQ ID NO: 6123 (or an amino acid sequence having at least about 75%, 80%, 85%, 90%, 95%, or 99% sequence identity to SEQ ID NO: 6123).

16. The antibody molecule of any of embodiments 5-15, wherein the antigen binding domain comprises a heavy chain variable region (VH) comprising a heavy chain framework region 1 (VHFWR1) amino acid sequence of SEQ ID NO: 6018 (or a sequence with no more than 1, 2, 3, 4, 5, or 6 mutations, e.g., substitutions, additions, or deletions, therefrom), a VHFWR2 amino acid sequence of SEQ ID NO: 6019 (or a sequence with no more than 1, 2, 3, 4, 5, or 6 mutations, e.g., substitutions, additions, or deletions, therefrom), a VHFWR3 amino acid sequence of SEQ ID NO: 6020 (or a sequence with no more than 1, 2, 3, 4, 5, or 6 mutations, e.g., substitutions, additions, or deletions, therefrom), or a VHFWR4 amino acid sequence of SEQ ID NO: 6021 (or a sequence with no more than 1, 2, 3, 4, 5, or 6 mutations, e.g., substitutions, additions, or deletions, therefrom).

17. The antibody molecule of embodiment 16, wherein the antigen binding domain comprises a heavy chain variable region (VH) comprising a heavy chain framework region 1 (VHFWR1) amino acid sequence of SEQ ID NO: 6018, a VHFWR2 amino acid sequence of SEQ ID NO: 6019, a VHFWR3 amino acid sequence of SEQ ID NO: 6020, or a VHFWR4 amino acid sequence of SEQ ID NO: 6021.

18. The antibody molecule of embodiment 17, wherein the antigen binding domain comprises a VH comprising the amino acid sequence of SEQ ID NO: 6124 (or an amino acid sequence having at least about 75%, 80%, 85%, 90%, 95%, or 99% sequence identity to SEQ ID NO: 6124).

19. The antibody molecule of any of embodiments 5-18, wherein the antigen binding domain comprises a heavy chain variable region (VH) comprising a heavy chain framework region 1 (VHFWR1) amino acid sequence of SEQ ID NO: 6022 (or a sequence with no more than 1, 2, 3, 4, 5, or 6 mutations, e.g., substitutions, additions, or deletions, therefrom), a VHFWR2 amino acid sequence of SEQ ID NO: 6023 (or a sequence with no more than 1, 2, 3, 4, 5, or 6 mutations, e.g., substitutions, additions, or deletions, therefrom), a VHFWR3 amino acid sequence of SEQ ID NO: 6024 (or a sequence with no more than 1, 2, 3, 4, 5, or 6 mutations, e.g., substitutions, additions, or deletions, therefrom), or a VHFWR4 amino acid sequence of SEQ ID NO: 6025 (or a sequence with no more than 1, 2, 3, 4, 5, or 6 mutations, e.g., substitutions, additions, or deletions, therefrom).

20. The antibody molecule of embodiment 19, wherein the antigen binding domain comprises a heavy chain variable region (VH) comprising a heavy chain framework region 1 (VHFWR1) amino acid sequence of SEQ ID NO: 6022, a VHFWR2 amino acid sequence of SEQ ID NO: 6023, a VHFWR3 amino acid sequence of SEQ ID NO: 6024, or a VHFWR4 amino acid sequence of SEQ ID NO: 6025.

21. The antibody molecule of embodiment 20, wherein the antigen binding domain comprises a VH comprising the amino acid sequence of SEQ ID NO: 6125 (or an amino acid sequence having at least about 75%, 80%, 85%, 90%, 95%, or 99% sequence identity to SEQ ID NO: 6125).

22. The antibody molecule of any of embodiments 5-21, wherein the antigen binding domain comprises a heavy chain variable region (VH) comprising a heavy chain framework region 1 (VHFWR1) amino acid sequence of SEQ ID NO: 6026 (or a sequence with no more than 1, 2, 3, 4, 5, or 6 mutations, e.g., substitutions, additions, or deletions, therefrom), a VHFWR2 amino acid sequence of SEQ ID NO: 6027 (or a sequence with no more than 1, 2, 3, 4, 5, or 6 mutations, e.g., substitutions, additions, or deletions, therefrom), a VHFWR3 amino acid sequence of SEQ ID NO: 6028 (or a sequence with no more than 1, 2, 3, 4, 5, or 6 mutations, e.g., substitutions, additions, or deletions, therefrom), or a VHFWR4 amino acid sequence of SEQ ID NO: 6029 (or a sequence with no more than 1, 2, 3, 4, 5, or 6 mutations, e.g., substitutions, additions, or deletions, therefrom).

23. The antibody molecule of embodiment 22, wherein the antigen binding domain comprises a heavy chain variable region (VH) comprising a heavy chain framework region 1 (VHFWR1) amino acid sequence of SEQ ID NO: 6026, a VHFWR2 amino acid sequence of SEQ ID NO: 6027, a VHFWR3 amino acid sequence of SEQ ID NO: 6028, or a VHFWR4 amino acid sequence of SEQ ID NO: 6029.

24. The antibody molecule of embodiment 23, wherein the antigen binding domain comprises a VH comprising the amino acid sequence of SEQ ID NO: 6126 (or an amino acid sequence having at least about 75%, 80%, 85%, 90%, 95%, or 99% sequence identity to SEQ ID NO: 6126).

25. The antibody molecule of any of embodiments 5-24, wherein the antigen binding domain comprises a heavy chain variable region (VH) comprising a heavy chain framework region 1 (VHFWR1) amino acid sequence of SEQ ID NO: 6030 (or a sequence with no more than 1, 2, 3, 4, 5, or 6 mutations, e.g., substitutions, additions, or deletions, therefrom), a VHFWR2 amino acid sequence of SEQ ID NO: 6032 (or a sequence with no more than 1, 2, 3, 4, 5, or 6 mutations, e.g., substitutions, additions, or deletions, therefrom), a VHFWR3 amino acid sequence of SEQ ID NO: 6033 (or a sequence with no more than 1, 2, 3, 4, 5, or 6 mutations, e.g., substitutions, additions, or deletions, therefrom), or a VHFWR4 amino acid sequence of SEQ ID NO: 6034 (or a sequence with no more than 1, 2, 3, 4, 5, or 6 mutations, e.g., substitutions, additions, or deletions, therefrom).

26. The antibody molecule of embodiment 25, wherein the antigen binding domain comprises a heavy chain variable region (VH) comprising a heavy chain framework region 1 (VHFWR1) amino acid sequence of SEQ ID NO: 6030, a VHFWR2 amino acid sequence of SEQ ID NO: 6032, a VHFWR3 amino acid sequence of SEQ ID NO: 6033, or a VHFWR4 amino acid sequence of SEQ ID NO: 6034.

27. The antibody molecule of embodiment 26, wherein the antigen binding domain comprises a VH comprising the amino acid sequence of SEQ ID NO: 6127 (or an amino acid sequence having at least about 75%, 80%, 85%, 90%, 95%, or 99% sequence identity to SEQ ID NO: 6127).

28. The antibody molecule of any of embodiments 5-27, wherein the antigen binding domain comprises a heavy chain variable region (VH) comprising a heavy chain framework region 1 (VHFWR1) amino acid sequence of SEQ ID NO: 6035 (or a sequence with no more than 1, 2, 3, 4, 5, or 6 mutations, e.g., substitutions, additions, or deletions, therefrom), a VHFWR2 amino acid sequence of SEQ ID NO: 6036 (or a sequence with no more than 1, 2, 3, 4, 5, or 6 mutations, e.g., substitutions, additions, or deletions, therefrom), a VHFWR3 amino acid sequence of SEQ ID NO: 6037 (or a sequence with no more than 1, 2, 3, 4, 5, or 6 mutations, e.g., substitutions, additions, or deletions, therefrom), or a VHFWR4 amino acid sequence of SEQ ID NO: 6038 (or a sequence with no more than 1, 2, 3, 4, 5, or 6 mutations, e.g., substitutions, additions, or deletions, therefrom).

29. The antibody molecule of embodiment 28, wherein the antigen binding domain comprises a heavy chain variable region (VH) comprising a heavy chain framework region 1 (VHFWR1) amino acid sequence of SEQ ID NO: 6035, a VHFWR2 amino acid sequence of SEQ ID NO: 6036, a VHFWR3 amino acid sequence of SEQ ID NO: 6037, or a VHFWR4 amino acid sequence of SEQ ID NO: 6038.

30. The antibody molecule of embodiment 29, wherein the antigen binding domain comprises a VH comprising the amino acid sequence of SEQ ID NO: 6128 (or an amino acid sequence ha3ving at least about 75%, 80%, 85%, 90%, 95%, or 99% sequence identity to SEQ ID NO: 6128).

31. The antibody molecule of any of embodiments 5, 6, or 13-30, wherein the antigen binding domain comprises a light chain variable region (VL) comprising a light chain framework region 1 (VLFWR1) amino acid sequence of SEQ ID NO: 6077 (or a sequence with no more than 1, 2, 3, 4, 5, or 6 mutations, e.g., substitutions, additions, or deletions, therefrom), a VLFWR2 amino acid sequence of SEQ ID NO: 6078 (or a sequence with no more than 1, 2, 3, 4, 5, or 6 mutations, e.g., substitutions, additions, or deletions, therefrom), a VLFWR3 amino acid sequence of SEQ ID NO: 6079 (or a sequence with no more than 1, 2, 3, 4, 5, or 6 mutations, e.g., substitutions, additions, or deletions, therefrom), or a VLFWR4 amino acid sequence of SEQ ID NO: 6080 (or a sequence with no more than 1, 2, 3, 4, 5, or 6 mutations, e.g., substitutions, additions, or deletions, therefrom).

32. The antibody molecule of embodiment 31, wherein the antigen binding domain comprises a light chain variable region (VL) comprising a light chain framework region 1 (VLFWR1) amino acid sequence of SEQ ID NO: 6077, a VLFWR2 amino acid sequence of SEQ ID NO: 6078, a VLFWR3 amino acid sequence of SEQ ID NO: 6079, or a VLFWR4 amino acid sequence of SEQ ID NO: 6080.

33. The antibody molecule of embodiment 32, wherein the antigen binding domain comprises a VL comprising the amino acid sequence of SEQ ID NO: 6137 (or an amino acid sequence having at least about 93%, 95%, or 99% sequence identity to SEQ ID NO: 6137).

34. The antibody molecule of any of embodiments 5, 6, or 13-30, wherein the antigen binding domain comprises a light chain variable region (VL) comprising a light chain framework region 1 (VLFWR1) amino acid sequence of SEQ ID NO: 6081 (or a sequence with no more than 1, 2, 3, 4, 5, or 6 mutations, e.g., substitutions, additions, or deletions, therefrom), a VLFWR2 amino acid sequence of SEQ ID NO: 6082 (or a sequence with no more than 1, 2, 3, 4, 5, or 6 mutations, e.g., substitutions, additions, or deletions, therefrom), a VLFWR3 amino acid sequence of SEQ ID NO: 6083 (or a sequence with no more than 1, 2, 3, 4, 5, or 6 mutations, e.g., substitutions, additions, or deletions, therefrom), or a VLFWR4 amino acid sequence of SEQ ID NO: 6084 (or a sequence with no more than 1, 2, 3, 4, 5, or 6 mutations, e.g., substitutions, additions, or deletions, therefrom).

35. The antibody molecule of embodiment 34, wherein the antigen binding domain comprises a light chain variable region (VL) comprising a light chain framework region 1 (VLFWR1) amino acid sequence of SEQ ID NO: 6081, a VLFWR2 amino acid sequence of SEQ ID NO: 6082, a VLFWR3 amino acid sequence of SEQ ID NO: 6083, or a VLFWR4 amino acid sequence of SEQ ID NO: 6084.

36. The antibody molecule of embodiment 35, wherein the antigen binding domain comprises a VL comprising the amino acid sequence of SEQ ID NO: 6138 (or an amino acid sequence having at least about 93%, 95%, or 99% sequence identity to SEQ ID NO: 6138).

37. The antibody molecule of any of embodiments 5, 6, or 13-30, wherein the antigen binding domain comprises a light chain variable region (VL) comprising a light chain framework region 1 (VLFWR1) amino acid sequence of SEQ ID NO: 6085 (or a sequence with no more than 1, 2, 3, 4, 5, or 6 mutations, e.g., substitutions, additions, or deletions, therefrom), a VLFWR2 amino acid sequence of SEQ ID NO: 6086 (or a sequence with no more than 1, 2, 3, 4, 5, or 6 mutations, e.g., substitutions, additions, or deletions, therefrom), a VLFWR3 amino acid sequence of SEQ ID NO: 6087 (or a sequence with no more than 1, 2, 3, 4, 5, or 6 mutations, e.g., substitutions, additions, or deletions, therefrom), or a VLFWR4 amino acid sequence of SEQ ID NO: 6088 (or a sequence with no more than 1, 2, 3, 4, 5, or 6 mutations, e.g., substitutions, additions, or deletions, therefrom).

38. The antibody molecule of embodiment 37, wherein the antigen binding domain comprises a light chain variable region (VL) comprising a light chain framework region 1 (VLFWR1) amino acid sequence of SEQ ID NO: 6085, a VLFWR2 amino acid sequence of SEQ ID NO: 6086, a VLFWR3 amino acid sequence of SEQ ID NO: 6087, or a VLFWR4 amino acid sequence of SEQ ID NO: 6088.

39. The antibody molecule of embodiment 38, wherein the antigen binding domain comprises a VL comprising the amino acid sequence of SEQ ID NO: 6139 (or an amino acid sequence having at least about 93%, 95%, or 99% sequence identity to SEQ ID NO: 6139).

40. The antibody molecule of any of embodiments 5, 6, or 13-30, wherein the antigen binding domain comprises a light chain variable region (VL) comprising a light chain framework region 1 (VLFWR1) amino acid sequence of SEQ ID NO: 6089 (or a sequence with no more than 1, 2, 3, 4, 5, or 6 mutations, e.g., substitutions, additions, or deletions, therefrom), a VLFWR2 amino acid sequence of SEQ ID NO: 6090 (or a sequence with no more than 1, 2, 3, 4, 5, or 6 mutations, e.g., substitutions, additions, or deletions, therefrom), a VLFWR3 amino acid sequence of SEQ ID NO: 6091 (or a sequence with no more than 1, 2, 3, 4, 5, or 6 mutations, e.g., substitutions, additions, or deletions, therefrom), or a VLFWR4 amino acid sequence of SEQ ID NO: 6092 (or a sequence with no more than 1, 2, 3, 4, 5, or 6 mutations, e.g., substitutions, additions, or deletions, therefrom).

41. The antibody molecule of embodiment 40, wherein the antigen binding domain comprises a light chain variable region (VL) comprising a light chain framework region 1 (VLFWR1) amino acid sequence of SEQ ID NO: 6089, a VLFWR2 amino acid sequence of SEQ ID NO: 6090, a VLFWR3 amino acid sequence of SEQ ID NO: 6091, or a VLFWR4 amino acid sequence of SEQ ID NO: 6092.

42. The antibody molecule of embodiment 41, wherein the antigen binding domain comprises a VL comprising the amino acid sequence of SEQ ID NO: 6140 (or an amino acid sequence having at least about 93%, 95%, or 99% sequence identity to SEQ ID NO: 6140).

43. The antibody molecule of any of embodiments 5, 6, or 13-30, wherein the antigen binding domain comprises a light chain variable region (VL) comprising a light chain framework region 1 (VLFWR1) amino acid sequence of SEQ ID NO: 6093 (or a sequence with no more than 1, 2, 3, 4, 5, or 6 mutations, e.g., substitutions, additions, or deletions, therefrom), a VLFWR2 amino acid sequence of SEQ ID NO: 6094 (or a sequence with no more than 1, 2, 3, 4, 5, or 6 mutations, e.g., substitutions, additions, or deletions, therefrom), a VLFWR3 amino acid sequence of SEQ ID NO: 6095 (or a sequence with no more than 1, 2, 3, 4, 5, or 6 mutations, e.g., substitutions, additions, or deletions, therefrom), or a VLFWR4 amino acid sequence of SEQ ID NO: 6096 (or a sequence with no more than 1, 2, 3, 4, 5, or 6 mutations, e.g., substitutions, additions, or deletions, therefrom).

44. The antibody molecule of embodiment 43, wherein the antigen binding domain comprises a light chain variable region (VL) comprising a light chain framework region 1 (VLFWR1) amino acid sequence of SEQ ID NO: 6093, a VLFWR2 amino acid sequence of SEQ ID NO: 6094, a VLFWR3 amino acid sequence of SEQ ID NO: 6095, or a VLFWR4 amino acid sequence of SEQ ID NO: 6096.

45. The antibody molecule of embodiment 44, wherein the antigen binding domain comprises a VL comprising the amino acid sequence of SEQ ID NO: 6141 (or an amino acid sequence having at least about 93%, 95%, or 99% sequence identity to SEQ ID NO: 6141).

46. An isolated antibody molecule that binds to NKp30, comprising:
(i) a heavy chain variable region (VH) comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions), and
(ii) a light chain variable region (VL) comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions).

47. The antibody molecule of embodiment 46, wherein the antigen binding domain comprises:
(i) a heavy chain variable region (VH) comprising a heavy chain complementarity determining region 1 (VHCDR1) amino acid sequence of SEQ ID NO: 6007, a VHCDR2 amino acid sequence of SEQ ID NO: 6008, and/or a VHCDR3 amino acid sequence of SEQ ID NO: 6009, and
(ii) a light chain variable region (VL) comprising a light chain complementarity determining region 1 (VLCDR1) amino acid sequence of SEQ ID NO: 6070, a VLCDR2 amino acid sequence of SEQ ID NO: 6071, and/or a VLCDR3 amino acid sequence of SEQ ID NO: 6072.

48. The antibody molecule of embodiments 46 or 47, wherein the antigen binding domain comprises:
(1) a heavy chain variable region (VH) comprising a heavy chain framework region 1 (VHFWR1) amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, or 6 mutations, e.g., substitutions, additions, or deletions, therefrom), or a VHFWR4 amino acid sequence of SEQ ID NO: 6013 (or a sequence with no more than 1, 2, 3, 4, 5, or 6 mutations, e.g., substitutions, additions, or deletions, therefrom), and/or
(2) a light chain variable region (VL) comprising a light chain framework region 1 (VLFWR1) amino acid sequence of SEQ ID NO: 6073 (or a sequence with no more than 1, 2, 3, 4, 5, or 6 mutations, e.g., substitutions, additions, or deletions, therefrom), a VLFWR2 amino acid sequence of SEQ ID NO: 6074 (or a sequence with no more than 1, 2, 3, 4, 5, or 6 mutations, e.g., substitutions, additions, or deletions, therefrom), a VLFWR3 amino acid sequence of SEQ ID NO: 6075 (or a sequence with no more than 1, 2, 3, 4, 5, or 6 mutations, e.g., substitutions, additions, or deletions, therefrom), or a VLFWR4 amino acid sequence of SEQ ID NO: 6076 (or a sequence with no more than 1, 2, 3, 4, 5, or 6 mutations, e.g., substitutions, additions, or deletions, therefrom).

49. The antibody molecule of embodiment 48, wherein the antigen binding domain comprises:
(1) a heavy chain variable region (VH) comprising a heavy chain framework region (VHFWR1) amino acid sequence of SEQ ID NO: 6010, a VHFWR2 amino acid sequence of SEQ ID NO: 6011, a VHFWR3 amino acid sequence of SEQ ID NO: 6012, or a VHFWR4 amino acid sequence of SEQ ID NO: 6013, and
(3) a light chain variable region (VL) comprising a light chain framework region 1 (VLFWR1) amino acid sequence of SEQ ID NO: 6073, a VLFWR2 amino acid sequence of SEQ ID NO: 6074, a VLFWRamino acid sequence of SEQ ID NO: 6075, or a VLFWR4 amino acid sequence of SEQ ID NO: 6076.

50. The antibody molecule of any one of embodiments 46-49, wherein the antigen binding domain comprises:
(i) a VH comprising the amino acid sequence of SEQ ID NO: 6122 (or an amino acid sequence having at least about 75%, 80%, 85%, 90%, 95%, or 99% sequence identity to SEQ ID NO: 6122), and/or
(ii) a VL comprising the amino acid sequence of SEQ ID NO: 6136 (or an amino acid sequence having at least about 93%, 95%, or 99% sequence identity to SEQ ID NO: 6136).

51. The antibody molecule of any of embodiments 46-50, wherein the antigen binding domain comprises a heavy chain comprising the amino acid sequence of SEQ ID NOs: 6151 or 6152 (or an amino acid sequence having at least about 75%, 80%, 85%, 90%, 95%, or 99% sequence identity to SEQ ID NOs: 6151 or 6152).

52. The antibody molecule of any of embodiments 46-51, wherein the antigen binding domain comprises a light chain comprising the amino acid sequence of SEQ ID NO: 6153 (or an amino acid sequence having at least about 75%, 80%, 85%, 90%, 95%, or 99% sequence identity to SEQ ID NO: 6153).

53. The antibody molecule of any of embodiments 46-51, wherein the antigen binding domain comprises a heavy chain comprising the amino acid sequence of SEQ ID NOs: 6151 or 6152 (or an amino acid sequence having at least about 75%, 80%, 85%, 90%, 95%, or 99% sequence identity to SEQ ID NOs: 6151 or 6152), and a light chain comprising the amino acid sequence of SEQ ID NO: 6153 (or an amino acid sequence having at least about 75%, 80%, 85%, 90%, 95%, or 99% sequence identity to SEQ ID NO: 6153).

54. The antibody molecule of embodiments 46 or 47, wherein the antigen binding domain comprises a heavy chain variable region (VH) comprising a heavy chain framework region 1 (VHFWR1) amino acid sequence of SEQ ID NO: 6039 (or a sequence with no more than 1, 2, 3, 4, 5, or 6 mutations, e.g., substitutions, additions, or deletions, therefrom), a VHFWR2 amino acid sequence of SEQ ID NO: 6040 (or a sequence with no more than 1, 2, 3, 4, 5, or 6 mutations, e.g., substitutions, additions, or deletions, therefrom), a VHFWR3 amino acid sequence of SEQ ID NO: 6041 (or a sequence with no more than 1, 2, 3, 4, 5, or 6 mutations, e.g., substitutions, additions, or deletions, therefrom), or a VHFWR4 amino acid sequence of SEQ ID NO: 6042 (or a sequence with no more than 1, 2, 3, 4, 5, or 6 mutations, e.g., substitutions, additions, or deletions, therefrom).

55. The antibody molecule of embodiment 54, wherein the antigen binding domain comprises a heavy chain variable region (VH) comprising a heavy chain framework region 1 (VHFWR1) amino acid sequence of SEQ ID NO: 6039, a VHFWR2 amino acid sequence of SEQ ID NO: 6040, a VHFWR3 amino acid sequence of SEQ ID NO: 6041, or a VHFWR4 amino acid sequence of SEQ ID NO: 6042.

56. The antibody molecule of embodiment 55, wherein the antigen binding domain comprises a VH comprising the amino acid sequence of SEQ ID NO: 6129 (or an amino acid sequence having at least about 75%, 80%, 85%, 90%, 95%, or 99% sequence identity to SEQ ID NO: 6129).

57. The antibody molecule of embodiments 46 or 47, wherein the antigen binding domain comprises a heavy chain variable region (VH) comprising a heavy chain framework region 1 (VHFWR1) amino acid sequence of SEQ ID NO: 6043 (or a sequence with no more than 1, 2, 3, 4, 5, or 6 mutations, e.g., substitutions, additions, or deletions, therefrom), a VHFWR2 amino acid sequence of SEQ ID NO: 6044 (or a sequence with no more than 1, 2, 3, 4, 5, or 6 mutations, e.g., substitutions, additions, or deletions, therefrom), a VHFWR3 amino acid sequence of SEQ ID NO: 6045 (or a sequence with no more than 1, 2, 3, 4, 5, or 6 mutations, e.g., substitutions, additions, or deletions, therefrom), or a VHFWR4 amino acid sequence of SEQ ID NO: 6046 (or a sequence with no more than 1, 2, 3, 4, 5, or 6 mutations, e.g., substitutions, additions, or deletions, therefrom).

58. The antibody molecule of embodiment 57, wherein the antigen binding domain comprises a heavy chain variable region (VH) comprising a heavy chain framework region 1 (VHFWR1) amino acid sequence of SEQ ID NO: 6043, a VHFWR2 amino acid sequence of SEQ ID NO: 6044, a VHFWR3 amino acid sequence of SEQ ID NO: 6045, or a VHFWR4 amino acid sequence of SEQ ID NO: 6046.

59. The antibody molecule of embodiment 58, wherein the antigen binding domain comprises a VH comprising the amino acid sequence of SEQ ID NO: 6130 (or an amino acid sequence having at least about 75%, 80%, 85%, 90%, 95%, or 99% sequence identity to SEQ ID NO: 6130).

60. The antibody molecule of any of embodiments 46 or 47, wherein the antigen binding domain comprises a heavy chain variable region (VH) comprising a heavy chain framework region 1 (VHFWR1) amino acid sequence of SEQ ID NO: 6047 (or a sequence with no more than 1, 2, 3, 4, 5, or 6 mutations, e.g., substitutions, additions, or deletions, therefrom), a VHFWR2 amino acid sequence of SEQ ID NO: 6048 (or a sequence with no more than 1, 2, 3, 4, 5, or 6 mutations, e.g., substitutions, additions, or deletions, therefrom), a VHFWR3 amino acid sequence of SEQ ID NO: 6049 (or a sequence with no more than 1, 2, 3, 4, 5, or 6 mutations, e.g., substitutions, additions, or deletions, therefrom), or a VHFWR4 amino acid sequence of SEQ ID NO: 6050 (or a sequence with no more than 1, 2, 3, 4, 5, or 6 mutations, e.g., substitutions, additions, or deletions, therefrom).

61. The antibody molecule of embodiment 60, wherein the antigen binding domain comprises a heavy chain variable region (VH) comprising a heavy chain framework region 1 (VHFWR1) amino acid sequence of SEQ ID NO: 6047, a VHFWR2 amino acid sequence of SEQ ID NO: 6048, a VHFWR3 amino acid sequence of SEQ ID NO: 6049, or a VHFWR4 amino acid sequence of SEQ ID NO: 6050.

62. The antibody molecule of embodiment 61, wherein the antigen binding domain comprises a VH comprising the amino acid sequence of SEQ ID NO: 6131 (or an amino acid sequence having at least about 75%, 80%, 85%, 90%, 95%, or 99% sequence identity to SEQ ID NO: 6131).

63. The antibody molecule of any of embodiments 46 or 47, wherein the antigen binding domain comprises a heavy chain variable region (VH) comprising a heavy chain framework region 1 (VHFWR1) amino acid sequence of SEQ ID NO: 6051 (or a sequence with no more than 1, 2, 3, 4, 5, or 6 mutations, e.g., substitutions, additions, or deletions, therefrom), a VHFWR2 amino acid sequence of SEQ ID NO: 6052 (or a sequence with no more than 1, 2, 3, 4, 5, or 6 mutations, e.g., substitutions, additions, or deletions, therefrom), a VHFWR3 amino acid sequence of SEQ ID NO: 6053 (or a sequence with no more than 1, 2, 3, 4, 5, or 6 mutations, e.g., substitutions, additions, or deletions, therefrom), or a VHFWR4 amino acid sequence of SEQ ID NO: 6054 (or a sequence with no more than 1, 2, 3, 4, 5, or 6 mutations, e.g., substitutions, additions, or deletions, therefrom).

64. The antibody molecule of embodiment 63, wherein the antigen binding domain comprises a heavy chain variable region (VH) comprising a heavy chain framework region 1 (VHFWR1) amino acid sequence of SEQ ID NO: 6051, a VHFWR2 amino acid sequence of SEQ ID NO: 6052, a VHFWR3 amino acid sequence of SEQ ID NO: 6053, or a VHFWR4 amino acid sequence of SEQ ID NO: 6054.

65. The antibody molecule of embodiment 64, wherein the antigen binding domain comprises a VH comprising the amino acid sequence of SEQ ID NO: 6132 (or an amino acid sequence having at least about 75%, 80%, 85%, 90%, 95%, or 99% sequence identity to SEQ ID NO: 6132).

66. The antibody molecule of any of embodiments 46 or 47, wherein the antigen binding domain comprises a heavy chain variable region (VH) comprising a heavy chain framework region 1 (VHFWR1) amino acid sequence of SEQ ID NO: 6055 (or a sequence with no more than 1, 2, 3, 4, 5, or 6 mutations, e.g., substitutions, additions, or deletions, therefrom), a VHFWR2 amino acid sequence of SEQ ID NO: 6056 (or a sequence with no more than 1, 2, 3, 4, 5, or 6 mutations, e.g., substitutions, additions, or deletions, therefrom), a VHFWR3 amino acid sequence of SEQ ID NO: 6057 (or a sequence with no more than 1, 2, 3, 4, 5, or 6 mutations, e.g., substitutions, additions, or deletions, therefrom), or a VHFWR4 amino acid sequence of SEQ ID NO: 6058 (or a sequence with no more than 1, 2, 3, 4, 5, or 6 mutations, e.g., substitutions, additions, or deletions, therefrom).

67. The antibody molecule of embodiment 66, wherein the antigen binding domain comprises a heavy chain variable region (VH) comprising a heavy chain framework region 1 (VHFWR1) amino acid sequence of SEQ ID NO: 6055, a VHFWR2 amino acid sequence of SEQ ID NO: 6056, a VHFWR3 amino acid sequence of SEQ ID NO: 6057, or a VHFWR4 amino acid sequence of SEQ ID NO: 6058.

68. The antibody molecule of embodiment 67, wherein the antigen binding domain comprises a VH comprising the amino acid sequence of SEQ ID NO: 6133 (or an amino acid sequence having at least about 75%, 80%, 85%, 90%, 95%, or 99% sequence identity to SEQ ID NO: 6133).

69. The antibody molecule of any of embodiments 46 or 47, wherein the antigen binding domain comprises a heavy chain variable region (VH) comprising a heavy chain framework region 1 (VHFWR1) amino acid sequence of SEQ ID NO: 6059 (or a sequence with no more than 1, 2, 3, 4, 5, or 6 mutations, e.g., substitutions, additions, or deletions, therefrom), a VHFWR2 amino acid sequence of SEQ ID NO: 6060 (or a sequence with no more than 1, 2, 3, 4, 5, or 6 mutations, e.g., substitutions, additions, or deletions, therefrom), a VHFWR3 amino acid sequence of SEQ ID NO: 6061 (or a sequence with no more than 1, 2, 3, 4, 5, or 6 mutations, e.g., substitutions, additions, or deletions, therefrom), or a VHFWR4 amino acid sequence of SEQ ID NO: 6062 (or a sequence with no more than 1, 2, 3, 4, 5, or 6 mutations, e.g., substitutions, additions, or deletions, therefrom).

70. The antibody molecule of embodiment 69, wherein the antigen binding domain comprises a heavy chain variable region (VH) comprising a heavy chain framework region 1 (VHFWR1) amino acid sequence of SEQ ID NO: 6059, a VHFWR2 amino acid sequence of SEQ ID NO: 6060, a VHFWR3 amino acid sequence of SEQ ID NO: 6061, or a VHFWR4 amino acid sequence of SEQ ID NO: 6062.

71. The antibody molecule of embodiment 70, wherein the antigen binding domain comprises a VH comprising the amino acid sequence of SEQ ID NO: 6134 (or an amino acid sequence having at least about 75%, 80%, 85%, 90%, 95%, or 99% sequence identity to SEQ ID NO: 6134).

72. The antibody molecule of any of embodiments 46, 47, or 54-71, wherein the antigen binding domain comprises a light chain variable region (VL) comprising a light chain framework region 1 (VLFWR1) amino acid sequence of SEQ ID NO: 6097 (or a sequence with no more than 1, 2, 3, 4, 5, or 6 mutations, e.g., substitutions, additions, or deletions, therefrom), a VLFWR2 amino acid sequence of SEQ ID NO: 6098 (or a sequence with no more than 1, 2, 3, 4, 5, or 6 mutations, e.g., substitutions, additions, or deletions, therefrom), a VLFWR3 amino acid sequence of SEQ ID NO: 6099 (or a sequence with no more than 1, 2, 3, 4, 5, or 6 mutations, e.g., substitutions, additions, or deletions, therefrom), or a VLFWR4 amino acid sequence of SEQ ID NO: 6100 (or a sequence with no more than 1, 2, 3, 4, 5, or 6 mutations, e.g., substitutions, additions, or deletions, therefrom).

73. The antibody molecule of embodiment 72, wherein the antigen binding domain comprises a light chain variable region (VL) comprising a light chain framework region 1 (VLFWR1) amino acid sequence of SEQ ID NO: 6097, a VLFWR2 amino acid sequence of SEQ ID NO: 6098, a VLFWR3 amino acid sequence of SEQ ID NO: 6099, or a VLFWR4 amino acid sequence of SEQ ID NO: 6100.

74. The antibody molecule of embodiment 73, wherein the antigen binding domain comprises a VL comprising the amino acid sequence of SEQ ID NO: 6142 (or an amino acid sequence having at least about 93%, 95%, or 99% sequence identity to SEQ ID NO: 6142).

75. The antibody molecule of any of embodiments 46, 47, or 54-74, wherein the antigen binding domain comprises a light chain variable region (VL) comprising a light chain framework region 1 (VLFWR1) amino acid sequence of SEQ ID NO: 6101 (or a sequence with no more than 1, 2, 3, 4, 5, or 6 mutations, e.g., substitutions, additions, or deletions, therefrom), a VLFWR2 amino acid sequence of SEQ ID NO: 6102 (or a sequence with no more than 1, 2, 3, 4, 5, or 6 mutations, e.g., substitutions, additions, or deletions, therefrom), a VLFWR3 amino acid sequence of SEQ ID NO: 6103 (or a sequence with no more than 1, 2, 3, 4, 5, or 6 mutations, e.g., substitutions, additions, or deletions, therefrom), or a VLFWR4 amino acid sequence of SEQ ID NO: 6104 (or a sequence with no more than 1, 2, 3, 4, 5, or 6 mutations, e.g., substitutions, additions, or deletions, therefrom).

76. The antibody molecule of embodiment 75, wherein the antigen binding domain comprises a light chain variable region (VL) comprising a light chain framework region 1 (VLFWR1) amino acid sequence of SEQ ID NO: 6101, a VLFWR2 amino acid sequence of SEQ ID NO: 6102, a VLFWR3 amino acid sequence of SEQ ID NO: 6103, or a VLFWR4 amino acid sequence of SEQ ID NO: 6104.

77. The antibody molecule of embodiment 76, wherein the antigen binding domain comprises a VL comprising the amino acid sequence of SEQ ID NO: 6143 (or an amino acid sequence having at least about 93%, 95%, or 99% sequence identity to SEQ ID NO: 6143).

78. The antibody molecule of any of embodiments 46, 47, or 54-77, wherein the antigen binding domain comprises a light chain variable region (VL) comprising a light chain framework region 1 (VLFWR1) amino acid sequence of SEQ ID NO: 6105 (or a sequence with no more than 1, 2, 3, 4, 5, or 6 mutations, e.g., substitutions, additions, or deletions, therefrom), a VLFWR2 amino acid sequence of SEQ ID NO: 6106 (or a sequence with no more than 1, 2, 3, 4, 5, or 6 mutations, e.g., substitutions, additions, or deletions, therefrom), a VLFWR3 amino acid sequence of SEQ ID NO: 6107 (or a sequence with no more than 1, 2, 3, 4, 5, or 6 mutations, e.g., substitutions, additions, or deletions, therefrom), or a VLFWR4 amino acid sequence of SEQ ID NO: 6108 (or a sequence with no more than 1, 2, 3, 4, 5, or 6 mutations, e.g., substitutions, additions, or deletions, therefrom).

79. The antibody molecule of embodiment 78, wherein the antigen binding domain comprises a light chain variable region (VL) comprising a light chain framework region 1 (VLFWR1) amino acid sequence of SEQ ID NO: 6105, a VLFWR2 amino acid sequence of SEQ ID NO: 6106, a VLFWR3 amino acid sequence of SEQ ID NO: 6107, or a VLFWR4 amino acid sequence of SEQ ID NO: 6108.

80. The antibody molecule of embodiment 79, wherein the antigen binding domain comprises a VL comprising the amino acid sequence of SEQ ID NO: 6144 (or an amino acid sequence having at least about 93%, 95%, or 99% sequence identity to SEQ ID NO: 6144).

81. The antibody molecule of any of embodiments 46, 47, or 54-80, wherein the antigen binding domain comprises a light chain variable region (VL) comprising a light chain framework region 1 (VLFWR1) amino acid sequence of SEQ ID NO: 6109 (or a sequence with no more than 1, 2, 3, 4, 5, or 6 mutations, e.g., substitutions, additions, or deletions, therefrom), a VLFWR2 amino acid sequence of SEQ ID NO: 6110 (or a sequence with no more than 1, 2, 3, 4, 5, or 6 mutations, e.g., substitutions, additions, or deletions, therefrom), a VLFWR3 amino acid sequence of SEQ ID NO: 6111 (or a sequence with no more than 1, 2, 3, 4, 5, or 6 mutations, e.g., substitutions, additions, or deletions, therefrom), or a VLFWR4 amino acid sequence of SEQ ID NO: 6112 (or a sequence with no more than 1, 2, 3, 4, 5, or 6 mutations, e.g., substitutions, additions, or deletions, therefrom).

82. The antibody molecule of embodiment 81, wherein the antigen binding domain comprises a light chain variable region (VL) comprising a light chain framework region 1 (VLFWR1) amino acid sequence of SEQ ID NO: 6109, a VLFWR2 amino acid sequence of SEQ ID NO: 6110, a VLFWR3 amino acid sequence of SEQ ID NO: 6111, or a VLFWR4 amino acid sequence of SEQ ID NO: 6112.

83. The antibody molecule of embodiment 78, wherein the antigen binding domain comprises a VL comprising the amino acid sequence of SEQ ID NO: 6145 (or an amino acid sequence having at least about 93%, 95%, or 99% sequence identity to SEQ ID NO: 6145).

84. The antibody molecule of any of embodiments 46, 47, or 54-83, wherein the antigen binding domain comprises a light chain variable region (VL) comprising a light chain framework region 1 (VLFWR1) amino acid sequence of SEQ ID NO: 6113 (or a sequence with no more than 1, 2, 3, 4, 5, or 6 mutations, e.g., substitutions, additions, or deletions, therefrom), a VLFWR2 amino acid sequence of SEQ ID NO: 6114 (or a sequence with no more than 1, 2, 3, 4, 5, or 6 mutations, e.g., substitutions, additions, or deletions, therefrom), a VLFWR3 amino acid sequence of SEQ ID NO: 6115 (or a sequence with no more than 1, 2, 3, 4, 5, or 6 mutations, e.g., substitutions, additions, or deletions, therefrom), or a VLFWR4 amino acid sequence of SEQ ID NO: 6116 (or a sequence with no more than 1, 2, 3, 4, 5, or 6 mutations, e.g., substitutions, additions, or deletions, therefrom).

85. The antibody molecule of embodiment 84, wherein the antigen binding domain comprises a light chain variable region (VL) comprising a light chain framework region 1 (VLFWR1) amino acid sequence of SEQ ID NO: 6113, a VLFWR2 amino acid sequence of SEQ ID NO: 6114, a VLFWR3 amino acid sequence of SEQ ID NO: 6115, or a VLFWR4 amino acid sequence of SEQ ID NO: 6116.

86. The antibody molecule of embodiment 85, wherein the antigen binding domain comprises a VL comprising the amino acid sequence of SEQ ID NO: 6146 (or an amino acid sequence having at least about 93%, 95%, or 99% sequence identity to SEQ ID NO: 6146).

87. The antibody molecule of any of embodiments 46, 47, or 54-86, wherein the antigen binding domain comprises a light chain variable region (VL) comprising a light chain framework region 1 (VLFWR1) amino acid sequence of SEQ ID NO: 6117 (or a sequence with no more than 1, 2, 3, 4, 5, or 6 mutations, e.g., substitutions, additions, or deletions, therefrom), a VLFWR2 amino acid sequence of SEQ ID NO: 6118 (or a sequence with no more than 1, 2, 3, 4, 5, or 6 mutations, e.g., substitutions, additions, or deletions, therefrom), a VLFWR3 amino acid sequence of SEQ ID NO: 6119 (or a sequence with no more than 1, 2, 3, 4, 5, or 6 mutations, e.g., substitutions, additions, or deletions, therefrom), or a VLFWR4 amino acid sequence of SEQ ID NO: 6120 (or a sequence with no more than 1, 2, 3, 4, 5, or 6 mutations, e.g., substitutions, additions, or deletions, therefrom).

88. The antibody molecule of embodiment 87, wherein the antigen binding domain comprises a light chain variable region (VL) comprising a light chain framework region 1 (VLFWR1) amino acid sequence of SEQ ID NO: 6117, a VLFWR2 amino acid sequence of SEQ ID NO: 6118, a VLFWR3 amino acid sequence of SEQ ID NO: 6119, or a VLFWR4 amino acid sequence of SEQ ID NO: 6120.

89. The antibody molecule of embodiment 88, wherein the antigen binding domain comprises a VL comprising the amino acid sequence of SEQ ID NO: 6147 (or an amino acid sequence having at least about 93%, 95%, or 99% sequence identity to SEQ ID NO: 6147).

90. The antibody molecule of any one of the preceding embodiments, wherein the antigen binding domain comprises:
(i) a VH comprising the amino acid sequence of SEQ ID NO: 6122 (or an amino acid sequence having at least about 75%, 80%, 85%, 90%, 95%, or 99% sequence identity to SEQ ID NO: 6122), and/or
(ii) a VL comprising the amino acid sequence of SEQ ID NO: 6136 (or an amino acid sequence having at least about 93%, 95%, or 99% sequence identity to SEQ ID NO: 6136).

91. A multispecific molecule comprising the antibody molecule of any of embodiments 1-90.

92. The multispecific molecule of embodiment 91, further comprising one, two, three, four or more of:
a. a tumor targeting moiety, e.g., as described herein;
b. a cytokine molecule, e.g., as described herein;
c. a T cell engager, e.g., as described herein; or
d. a stromal modifying moiety, e.g., as described herein.

93. The multispecific molecule of embodiment 91, further comprising a binding specificity that binds to an autoreactive T cell, e.g., an antigen present on the surface of an autoreactive T cell that is associated with the inflammatory or autoimmune disorder.

94. The multispecific molecule of embodiment 91, further comprising a binding specificity that binds to an infected cell, e.g., a viral or bacterial infected cell.

95. The antibody molecule, or the multispecific molecule of any of the preceding embodiments, which is a monospecific antibody molecule, a bispecific antibody molecule, or a trispecific antibody molecule.

96. The antibody molecule, or the multispecific molecule of any of the preceding embodiments, which is a monovalent antibody molecule, a bivalent antibody molecule, or a trivalent antibody molecule.

97. The antibody molecule, or the multispecific molecule of any of the preceding embodiments, which is a full antibody (e.g., an antibody that includes at least one, and preferably two, complete heavy chains, and at least one, and preferably two, complete light chains), or an antigen-binding fragment (e.g., a Fab, F(ab′)₂, Fv, a single chain Fv, a single domain antibody, a diabody (dAb), a bivalent antibody, or bispecific antibody or fragment thereof, a single domain variant thereof, or a camelid antibody).

98. The antibody molecule, or the multispecific molecule of any of the preceding embodiments, which comprises a heavy chain constant region chosen from IgG1, IgG2, IgG3, or IgG4, or a fragment thereof.

99. The antibody molecule, or the multispecific molecule of any of the preceding embodiments, which comprises a light chain constant region chosen from the light chain constant regions of kappa or lambda, or a fragment thereof.

100. The antibody molecule, or the multispecific molecule of any of the preceding embodiments, wherein the immunoglobulin chain constant region (e.g., Fc region) is altered, e.g., mutated, to increase or decrease one or more of: Fc receptor binding, antibody glycosylation, the number of cysteine residues, effector cell function, or complement function.

101. The antibody molecule, or the multispecific molecule of any of the preceding embodiments, wherein an interface of a first and second immunoglobulin chain constant regions (e.g., Fc region) is altered, e.g., mutated, to increase or decrease dimerization, e.g., relative to a non-engineered interface.

102. The antibody molecule or the multispecific molecule of embodiment 101, wherein the dimerization of the immunoglobulin chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface.

103. The antibody molecule or the multispecific molecule of embodiment 101 or 102, wherein the immunoglobulin chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1.

104. The antibody molecule or the multispecific molecule of embodiment 103, wherein the immunoglobulin chain constant region (e.g., Fc region) comprises an amino acid substitution chosen from: T366S, L368A, or Y407V (e.g., corresponding to a cavity or hole), or T366W (e.g., corresponding to a protuberance or knob), or a combination thereof.

105. The antibody molecule or the multispecific molecule of any of embodiments 1-104, further comprising a linker, e.g., a linker between one or more of: the targeting moiety and the cytokine molecule or the stromal modifying moiety, the targeting moiety and the immune cell engager, the cytokine molecule or the stromal modifying moiety, and the immune cell engager, the cytokine molecule or the stromal modifying moiety and the immunoglobulin chain constant region (e.g., the Fc region), the targeting moiety and the immunoglobulin chain constant region, or the immune cell engager and the immunoglobulin chain constant region.

106. The antibody molecule or the multispecific molecule of embodiment 105, wherein the linker is selected from: a cleavable linker, a non-cleavable linker, a peptide linker, a flexible linker, a rigid linker, a helical linker, or a non-helical linker.

107. The antibody molecule or the multispecific molecule of embodiment 106, wherein the linker is a peptide linker.

108. The antibody molecule or the multispecific molecule of embodiment 107, wherein the peptide linker comprises Gly and Ser.

109. An isolated nucleic acid molecule, which comprises the nucleotide sequence encoding any of the antibody molecules or multispecific or multifunctional molecules described herein, or a nucleotide sequence substantially homologous thereto (e.g., at least 95% to 99.9% identical thereto).

110. An isolated nucleic acid encoding the antibody molecule or the multispecific molecule of any of embodiments 1-108.

111. A vector, e.g., an expression vector, comprising one or more of the nucleic acid molecules of any of embodiments 109 or 110.

112. A host cell comprising the nucleic acid molecule or the vector of embodiment 111.

113. A method of making, e.g., producing, the antibody molecule or the multispecific or multifunctional molecule polypeptide of any of embodiments 1-108, comprising culturing the host cell of embodiment 112, under suitable conditions, e.g., conditions suitable for gene expression and/or homo- or heterodimerization.

114. A pharmaceutical composition comprising the antibody molecule or the multispecific or multifunctional molecule polypeptide of any of embodiments 1-108 and a pharmaceutically acceptable carrier, excipient, or stabilizer.

115. A method of treating a cancer, comprising administering to a subject in need thereof the antibody molecule or the multispecific or multifunctional molecule polypeptide of any of the preceding embodiments, wherein the multispecific antibody is administered in an amount effective to treat the cancer.

116. The antibody molecule or the multispecific or multifunctional molecule polypeptide of any of the preceding embodiments for use in treating cancer.

117. The method of embodiment 115 or the use of embodiment 116, wherein the cancer is a solid tumor cancer, or a metastatic lesion.

118. The method of embodiment 117 or the use of embodiment 117, wherein the solid tumor cancer is one or more of pancreatic (e.g., pancreatic adenocarcinoma), breast, colorectal, lung (e.g., small or non-small cell lung cancer), skin, ovarian, or liver cancer.

119. The method of embodiment 115 or the use of embodiment 116, wherein the cancer is a hematological cancer.

120. The method of any of embodiments 115 or 116-119 or the use of any of embodiments 116-119, further comprising administering a second therapeutic treatment.

121. The method of embodiment 120 or the use of embodiment 120, wherein the second therapeutic treatment comprises a therapeutic agent (e.g., a chemotherapeutic agent, a biologic agent, hormonal therapy), radiation, or surgery.

122. The method of embodiment 121 or the use of embodiment 121, wherein the therapeutic agent is selected from: a chemotherapeutic agent, or a biologic agent.

123. A method of treating an autoimmune or an inflammatory disorder, comprising administering to a subject in need thereof the antibody molecule or the multispecific or multifunctional molecule polypeptide of any of the preceding embodiments, wherein the multispecific antibody is administered in an amount effective to treat the autoimmune or the inflammatory disorder.

124. The antibody molecule or the multispecific or multifunctional molecule polypeptide of any of the preceding embodiments for use in treating an autoimmune or an inflammatory disorder.

125. A method of treating an infectious disorder, comprising administering to a subject in need thereof the antibody molecule or the multispecific or multifunctional molecule polypeptide of any of the preceding embodiments, wherein the multispecific antibody is administered in an amount effective to treat the infectious disorder.

126. The antibody molecule or the multispecific or multifunctional molecule polypeptide of any of the preceding embodiments for use in treating an infectious disorder.

### Exemplary Embodiment 3

1. An isolated antibody molecule that binds to NKp30, comprising:
(a) a heavy chain complementarity determining region 1 (VHCDR1), a heavy chain complementarity determining region 2 (VHCDR2), and/or a heavy chain complementarity determining region 3 (VHCDR3) described in Table 8A, or a sequence with no more than 1, 2, 3, or 4 mutations, e.g., substitutions, additions, or deletions; and/or
(b) a light chain complementarity determining region 1 (VLCDR1), a light chain complementarity determining region 2 (VLCDR2), and/or a light chain complementarity determining region 3 (VLCDR3) described in Table 8B, or a sequence with no more than 1, 2, 3, or 4 mutations, e.g., substitutions, additions, or deletions.

2. The antibody molecule of embodiment 1, wherein the VHCDR1 comprises the amino acid sequence of any of SEQ ID NOs: 19, 33, 47, 61, 7397, 89, 103, or 116, or an amino acid sequence with no more than 1, 2, 3, or 4 mutations, e.g., substitutions, additions, or deletions.

3. The antibody molecule of embodiment 1 or 2, wherein the VHCDR2 comprises the amino acid sequence of any of SEQ ID NOs: 21, 35, 49, 63, 7399, 91, 105, or 118, or an amino acid sequence with no more than 1, 2, 3, or 4 mutations, e.g., substitutions, additions, or deletions.

4. The antibody molecule of any of embodiments 1-3, wherein the VHCDR3 comprises the amino acid sequence of any of SEQ ID NOs: 23, 37, 51, 65, 79, 93, 107, or 120, or an amino acid sequence with no more than 1, 2, 3, or 4 mutations, e.g., substitutions, additions, or deletions.

5. The antibody molecule of any of embodiments 1-4, wherein the VLCDR1 comprises the amino acid sequence of any of SEQ ID NOs: 26, 40, 54, 68, 82, 96, 110, or 123, or an amino acid sequence with no more than 1, 2, 3, or 4 mutations, e.g., substitutions, additions, or deletions.

6. The antibody molecule of any of embodiments 1-5, wherein the VLCDR2 comprises the amino acid sequence of any of SEQ ID NOs: 28, 7393, 56, 70, 84, 98, 112, or 125, or an amino acid sequence with no more than 1, 2, 3, or 4 mutations, e.g., substitutions, additions, or deletions.

7. The antibody molecule of any of embodiments 1-6, wherein the VLCDR3 comprises the amino acid sequence of any of SEQ ID NOs: 30, 7395, 58, 72, 86, 100, 113, or 127, or an amino acid sequence with no more than 1, 2, 3, or 4 mutations, e.g., substitutions, additions, or deletions.

8. The antibody molecule of any of embodiments 1-7, comprising:
(a) a heavy chain framework region 1 (VHFWR1), a heavy chain framework region 2 (VHFWR1), a heavy chain framework region 3 (VHFWR3), and/or a heavy chain framework region 4 (VHFWR4) described in Table 8A, or an amino acid sequence with no more than 1, 2, 3, or 4 mutations, e.g., substitutions, additions, or deletions; and/or
(b) a light chain framework region 1 (VLFWR1), a light chain framework region 2 (VLFWR1), a light chain framework region 3 (VLFWR3), and/or a light chain framework region 4 (VLFWR4) described in Table 8B, or an amino acid sequence with no more than 1, 2, 3, or 4 mutations, e.g., substitutions, additions, or deletions.

9. The antibody molecule of any of embodiments 1-8, comprising:
(a) a heavy chain variable region (VH) described in Table 9, e.g., a VH comprising the amino acid sequence of any of SEQ ID NOs: 1-8, or an amino acid sequence having at least about 75%, 80%, 85%, 90%, 95%, or 99% sequence identity thereto; and/or
(b) a light chain variable region (VL) described in Table 9, e.g., a VL comprising the amino acid sequence of any of SEQ ID NOs: 9-16, or an amino acid sequence having at least about 75%, 80%, 85%, 90%, 95%, or 99% sequence identity thereto.

10. The antibody molecule of any of embodiments 1-9, comprising an amino acid sequence described in Table 10, e.g., the amino acid sequence of any of SEQ ID NOs: 17, and 7386-7392, or an amino acid sequence having at least about 75%, 80%, 85%, 90%, 95%, or 99% sequence identity thereto.

11. A multispecific molecule comprising the antibody molecule of any of embodiments 1-10.

12. The multispecific molecule of embodiment 11, further comprising one, two, three, four or more of:
a. a tumor targeting moiety, e.g., as described herein;
b. a cytokine molecule, e.g., as described herein;
c. a T cell engager, e.g., as described herein; or
d. a stromal modifying moiety, e.g., as described herein.

13. The multispecific molecule of embodiment 11, further comprising a binding specificity that binds to an autoreactive T cell, e.g., an antigen present on the surface of an autoreactive T cell that is associated with the inflammatory or autoimmune disorder.

14. The multispecific molecule of embodiment 11, further comprising a binding specificity that binds to an infected cell, e.g., a viral or bacterial infected cell.

15. The antibody molecule of any of embodiments 1-10, or the multispecific molecule of any of embodiments 11-14, which is a monospecific antibody molecule, a bispecific antibody molecule, or a trispecific antibody molecule.

16. The antibody molecule of any of embodiments 1-10, or the multispecific molecule of any of embodiments 11-14, which is a monovalent antibody molecule, a bivalent antibody molecule, or a trivalent antibody molecule.

17. The antibody molecule of any of embodiments 1-10, 15, or 16, or the multispecific molecule of any of embodiments 11-15, which is a full antibody (e.g., an antibody that includes at least one, and preferably two, complete heavy chains, and at least one, and preferably two, complete light chains), or an antigen-binding fragment (e.g., a Fab, F(ab′)₂, Fv, a single chain Fv, a single domain antibody, a diabody (dAb), a bivalent antibody, or bispecific antibody or fragment thereof, a single domain variant thereof, or a camelid antibody).

18. The antibody molecule or the multispecific molecule of any of embodiments 1-17, which comprises a heavy chain constant region chosen from IgG1, IgG2, IgG3, or IgG4, or a fragment thereof.

19. The antibody molecule or the multispecific molecule of any of embodiments 1-18, which comprises a light chain constant region chosen from the light chain constant regions of kappa or lambda, or a fragment thereof.

20. The antibody molecule or the multispecific molecule of any of embodiments 1-19, wherein the immunoglobulin chain constant region (e.g., Fc region) is altered, e.g., mutated, to increase or decrease one or more of: Fc receptor binding, antibody glycosylation, the number of cysteine residues, effector cell function, or complement function.

21. The antibody molecule or the multispecific molecule of any of embodiments 1-20, wherein an interface of a first and second immunoglobulin chain constant regions (e.g., Fc region) is altered, e.g., mutated, to increase or decrease dimerization, e.g., relative to a non-engineered interface.

22. The antibody molecule or the multispecific molecule of embodiment 21, wherein the dimerization of the immunoglobulin chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface.

23. The antibody molecule or the multispecific molecule of embodiment 21 or 22, wherein the immunoglobulin chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1.

24. The antibody molecule or the multispecific molecule of embodiment 21, wherein the immunoglobulin chain constant region (e.g., Fc region) comprises an amino acid substitution chosen from: T366S, L368A, or Y407V (e.g., corresponding to a cavity or hole), or T366W (e.g., corresponding to a protuberance or knob), or a combination thereof.

25. The antibody molecule or the multispecific molecule of any of embodiments 1-24, further comprising a linker, e.g., a linker between one or more of: the targeting moiety and the cytokine molecule or the stromal modifying moiety, the targeting moiety and the immune cell engager, the cytokine molecule or the stromal modifying moiety, and the immune cell engager, the cytokine molecule or the stromal modifying moiety and the immunoglobulin chain constant region (e.g., the Fc region), the targeting moiety and the immunoglobulin chain constant region, or the immune cell engager and the immunoglobulin chain constant region.

26. The antibody molecule or the multispecific molecule of embodiment 25, wherein the linker is selected from: a cleavable linker, a non-cleavable linker, a peptide linker, a flexible linker, a rigid linker, a helical linker, or a non-helical linker.

27. The antibody molecule or the multispecific molecule of embodiment 26, wherein the linker is a peptide linker.

28. The antibody molecule or the multispecific molecule of embodiment 27, wherein the peptide linker comprises Gly and Ser.

29. An isolated nucleic acid molecule, which comprises the nucleotide sequence encoding any of the antibody molecules or multispecific or multifunctional molecules described herein, or a nucleotide sequence substantially homologous thereto (e.g., at least 95% to 99.9% identical thereto).

30. An isolated nucleic acid encoding the antibody molecule or the multispecific molecule of any of embodiments 1-29.

31. A vector, e.g., an expression vector, comprising one or more of the nucleic acid molecules of embodiment 29 or 30.

32. A host cell comprising the nucleic acid molecule or the vector of any one of embodiments 29-31.

33. A method of making, e.g., producing, the antibody molecule or the multispecific or multifunctional molecule polypeptide of any of embodiments 1-28, comprising culturing the host cell of embodiment 32, under suitable conditions, e.g., conditions suitable for gene expression and/or homo- or heterodimerization.

34. A pharmaceutical composition comprising the antibody molecule or the multispecific or multifunctional molecule polypeptide of any of embodiments 1-28 and a pharmaceutically acceptable carrier, excipient, or stabilizer.

35. A method of treating a cancer, comprising administering to a subject in need thereof the antibody molecule or the multispecific or multifunctional molecule polypeptide of any of embodiments 1-12 or 15-28, wherein the multispecific antibody is administered in an amount effective to treat the cancer.

36. The antibody molecule or the multispecific or multifunctional molecule polypeptide of any of embodiments 1-12 or 15-28 for use in treating cancer.

37. The method of embodiment 35 or the use of embodiment 36, wherein the cancer is a solid tumor cancer, or a metastatic lesion.

38. The method of embodiment 37 or the use of embodiment 37, wherein the solid tumor cancer is one or more of pancreatic (e.g., pancreatic adenocarcinoma), breast, colorectal, lung (e.g., small or non-small cell lung cancer), skin, ovarian, or liver cancer.

39. The method of embodiment 35 or the use of embodiment 36, wherein the cancer is a hematological cancer.

40. The method of any of embodiments 35 or 37-39 or the use of any of embodiments 36-39, further comprising administering a second therapeutic treatment.

41. The method of embodiment 40 or the use of embodiment 40, wherein the second therapeutic treatment comprises a therapeutic agent (e.g., a chemotherapeutic agent, a biologic agent, hormonal therapy), radiation, or surgery.

42. The method of embodiment 41 or the use of embodiment 41, wherein the therapeutic agent is selected from: a chemotherapeutic agent, or a biologic agent.

43. A method of treating an autoimmune or an inflammatory disorder, comprising administering to a subject in need thereof the antibody molecule or the multispecific or multifunctional molecule polypeptide of any of embodiments 1-11, 13, or 15-28, wherein the multispecific antibody is administered in an amount effective to treat the autoimmune or the inflammatory disorder.

44. The antibody molecule or the multispecific or multifunctional molecule polypeptide of any of embodiments 1-11, 13, or 15-28 for use in treating an autoimmune or an autoinflammatory disorder.

45. A method of treating an infectious disorder, comprising administering to a subject in need thereof the antibody molecule or the multispecific or multifunctional molecule polypeptide of any of embodiments 1-11 or 14-28, wherein the multispecific antibody is administered in an amount effective to treat the infectious disorder.

46. The antibody molecule or the multispecific or multifunctional molecule polypeptide of any of embodiments 1-11 or 14-28 for use in treating an infectious disorder.

What is claimed is:

1-45. (canceled)

46. A composition comprising an antibody molecule that binds to NKp30, wherein the antibody molecule comprises:
(a)(i) a heavy chain variable region (VH) comprising a heavy chain complementarity determining region 1 (VHCDR1) comprising the sequence of SEQ ID NO: 19, a heavy chain complementarity determining region 2 (VHCDR2) comprising the sequence of SEQ ID NO: 21, and a heavy chain complementarity determining region 3 (VHCDR3) comprising the sequence of SEQ ID NO: 23; and
(ii) a light chain variable region (VL) comprising a light chain complementarity determining region 1 (VLCDR1) comprising the sequence of SEQ ID NO: 26, a light chain complementarity determining region 2 (VLCDR2) comprising the sequence of SEQ ID NO: 28, and a light chain complementarity determining region 3 (VLCDR3) comprising the sequences of SEQ ID NO: 30, SEQ ID NO: 7395, or SEQ ID NO: 113; or
(b)(i) a VH comprising a VHCDR1 comprising the sequence of SEQ ID NO: 19, a VHCDR2 comprising the sequence of SEQ ID NO: 7399, and a VHCDR3 comprising the sequence of the sequence of SEQ ID NO: 23; and
(ii) a VL comprising a VLCDR1 comprising the sequence of SEQ ID NO: 26, a VLCDR2 comprising the sequence of SEQ ID NO: 28, and a VLCDR3 comprising the sequences of SEQ ID NO: 30, SEQ ID NO: 7395, SEQ ID NO: 86, or SEQ ID NO: 113.

47. The composition of claim 46, wherein the antibody molecule comprises:
(i) a VH comprising an amino acid sequence having at least 80% sequence identity to the sequence of SEQ ID NO: 1 or SEQ ID NO: 2;
(ii) a VL comprising an amino acid sequence having at least 80% sequence identity to any one sequence selected from the group consisting of SEQ ID NOs: 9-16; or
(iii) any combination thereof.

48. The composition of claim 46, wherein the antibody molecule comprises:
(a) a VH comprising the amino acid sequence of SEQ ID NO: 1, and a VL comprising any one of the amino acid sequences selected from the group consisting of SEQ ID NOs: 9-12, 15, and 16; or
(b) a VH comprising the amino acid sequence of SEQ ID NO: 2, and a VL comprising any one of the amino acid sequences selected from the group consisting of SEQ ID NOs: 9-16.

49. The composition of claim 46, wherein the antibody molecule comprises an amino acid sequence having at least 80% sequence identity to any one sequence selected from the group consisting of SEQ ID NOs: 17 and 7386-7392.

50. The composition of claim 46, wherein the antibody molecule comprises any one amino acid sequence selected from the group consisting of SEQ ID NOs: 17 and 7386-7392.

51. The composition of claim 46, wherein the antibody molecule is a full antibody or an antigen-binding fragment thereof, a bivalent antibody, or a bispecific antibody,
wherein the antigen-binding fragment is a Fab, F(ab′)2, Fv, a single chain Fv, a single domain antibody, a diabody, or a camelid antibody.

52. The composition of claim 46, wherein the antibody molecule is a multifunctional antibody molecule, a multispecific antibody molecule, or a combination thereof.

53. The composition of claim 52, wherein the antibody molecule comprises:
(i) an immunoglobulin heavy chain constant region selected from the group consisting of IgG1 heavy chain constant region or fragment thereof, IgG2 heavy chain constant region or fragment thereof, IgG3 heavy chain constant region or fragment thereof, and IgG4 heavy chain constant region or fragment thereof;
(ii) a light chain constant region selected from the group consisting of the kappa light chain constant region or fragment thereof, and the lambda light chain constant region or fragment thereof; or
(iii) any combination thereof.

54. The composition of claim 53, wherein the immunoglobulin heavy chain constant region is altered to increase or decrease one or more of: Fc receptor binding, antibody glycosylation, the number of cysteine residues, effector cell function, or complement function.

55. The composition of claim 53, wherein the antibody molecule comprises a first immunoglobulin heavy chain constant region and a second immunoglobulin heavy chain constant region,
wherein the first immunoglobulin heavy chain constant region comprises a first Fc region, and the second immunoglobulin heavy chain constant region comprises a second Fc region, and
wherein dimerization of the first immunoglobulin heavy chain constant region and the second immunoglobulin heavy chain constant region is enhanced by providing an Fc interface of the first Fc region and the second Fc region with one or more of: a paired cavity-protuberance, an electrostatic interaction, or a strand-exchange, such that a greater ratio of heteromultimer:homomultimer forms relative to a non-engineered interface.

56. The composition of claim 55, wherein the first Fc region comprises an amino acid substitution selected from the group consisting of T366S, L368A, Y407V, and any combination thereof, and the second Fc region comprises an amino acid substitution of T366W, or the first Fc region comprises an amino acid substitution of T366W, and the second Fc region comprises an amino acid substitution selected from the group consisting of T366S, L368A, Y407V, and any combination thereof.

57. The composition of claim 52, wherein the antibody molecule further comprises a cytokine molecule.

58. The composition of claim 57, wherein the cytokine molecule is interleukin-2 (IL-2) molecule or functional variant thereof, interleukin-12 (IL-12) molecule or functional variant thereof, interleukin-15 (IL-15) molecule or functional variant thereof, interleukin-18 (IL-18) molecule or functional variant thereof, interleukin-21 (IL-21) molecule or functional variant thereof, interleukin-7 (IL-7) molecule or functional variant thereof, or interferon gamma molecule or functional variant thereof.

59. The composition of claim 52, wherein the antibody molecule further comprises a T cell engager.

60. The composition of claim 52, wherein the antibody molecule further comprises a targeting moiety selected from the group consisting of a tumor targeting moiety, a targeting moiety that targets an autoreactive T cell, and a targeting moiety that targets an infected cell.

61. The composition of claim 60, wherein:
(i) the targeting moiety that targets an autoreactive T cell binds to an antigen present on the surface of the autoreactive T cell, and
wherein the antigen present on the surface of the autoreactive T cell is associated with an inflammatory or autoimmune disorder; or
(ii) the targeting moiety that targets the infected cell binds to an antigen associated with a viral infection or a bacterial infection.

62. The composition of claim 61, wherein the antigen present on the surface of the autoreactive T cell is selected from the group consisting of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, and CD226.

63. The composition of claim 52, wherein the antibody molecule comprises one or more of:
(a) a tumor targeting moiety;
(b) a cytokine molecule;
(c) a T cell engager; or
(d) a stromal modifying moiety.

64. A recombinant polynucleotide comprising a sequence encoding the antibody molecule of the composition of claim 46.

65. A method of making the antibody molecule of the composition of claim 46, comprising culturing a host cell comprising a recombinant polynucleotide comprising a sequence encoding the antibody molecule of the composition under conditions suitable for gene expression and/or homo- or heterodimerization.

66. A pharmaceutical composition comprising the composition of claim 46, and a pharmaceutically acceptable carrier, excipient, diluent, or stabilizer.

67. A method of treating a disease or disorder in a subject in need thereof comprising administering to the subject an effective amount of the composition of claim 46,
wherein the disease or disorder is cancer, an autoimmune disorder or an inflammatory disorder, or an infectious disorder; and
wherein the administering is effective to treat the disease or disorder in the subject.

68. The method of claim 67, wherein:
(a) the cancer is a solid tumor cancer, a hematological cancer, or a metastatic cancer; or
(b) the infectious disorder is associated with a viral infection or a bacterial infection.

69. The method of claim 68, wherein the solid tumor cancer is one or more of pancreatic cancer, breast cancer, colorectal cancer, lung cancer, skin cancer, ovarian cancer, or liver cancer.
